(19) United States
(12) Patent Application Publication
(10) Pub. No.: US 2019/0376046 A1
(43) Pub. Date: Dec. 12, 2019

(54) METHODS FOR MANIPULATING CELL FATE

(71) Applicant: The McLean Hospital Corporation, Belmont, MA (US)

(72) Inventor: Kwang-Soo Kim, Lexington, MA (US)

(21) Appl. No.: 16/483,107

(22) PCT Filed: Feb. 2, 2018
(86) PCT No.: PCT/US18/16644

§ 371 Date: Aug. 2, 2019

Related U.S. Application Data
(60) Provisional application No. 62/454,254, filed on Feb. 3, 2017.

(51) Int. Cl. C12N 5/074; C12N 15/113; A61K 35/28

(52) U.S. Cl. CPC C12N 5/0696; C12N 15/1135; A61K 35/28; C12N 2501/65; C12N 2510/00; C12N 2501/604; C12N 2501/603; C12N 2501/602; C12N 2501/605; C12N 2501/608; C12N 2501/606

(57) ABSTRACT

Disclosed herein are methods of generating induced pluripotent stem cells. The method involves providing a somatic or non-embryonic cell population, contacting the somatic or non-embryonic cell population with a quantity of at least one reprogramming factor, an agent that downmodulates SIRT2, and/or an agent that upmodulates SIRT1, and culturing the somatic or non-embryonic cells for a period of time sufficient to generate at least one induced pluripotent stem cell. Methods for differentiating a cell by upmodulating SIRT2 and/or downmodulating SIRT1 are also provided herein. Also disclosed are cell lines and pharmaceutical compositions generated by use of the methods.

### CROSS-REFERENCE TO RELATED APPLICATION

This application claims benefit under 35 U.S.C. § 119(e) of the U.S. Provisional Application No. 62/454,254 filed Feb. 3, 2017, the contents of which are incorporated herein by reference in their entirety.

### GOVERNMENT SUPPORT

This invention was made with Government support under Grant Nos. NS084869, NS070577, and GM101420 awarded by the National Institutes of Health. The Government has certain rights in the invention.

### FIELD OF THE INVENTION

The field of the invention relates to the field of regenerative medicine.

### BACKGROUND

In the early twentieth century, Otto Warburg observed a metabolic switch in transformed cells compared to normal cells from oxidative phosphorylation (OXPHOS) to glycolysis, even in the presence of high levels of oxygen¹. Interestingly, recent studies showed that the metabolism of different types of stem cells, in particular primed pluripotent stem cells (e.g., hESCs and hiPSCs), is also biased towards glycolysis rather than OXPHOS, exhibiting a Warburg-like effect⁷. Indeed, more recent studies showed that in primed hPSCs this metabolic switch from OXPHOS to glycolysis is critical for bioenergetics, biosynthetic capacity, and/or epigenetic regulation in hPSCs⁸⁻¹², which was further supported by metabolomics analyses^{11, 13}. Unlike hESCs and hiPSCs that represent a primed state, mouse ESCs are known to be at a naive state and energetically bivalent, and can dynamically switch from glycolysis to OXPHOS on demand⁹. Thus, these studies suggest that metabolic reprogramming is intimately linked to stem cell identity during induced pluripotency. However, whether it is causative, or merely reflective of identity is unknown.

Despite many efforts to optimize reprogramming techniques to manipulate cell fate (e.g., induce pluripotency or produce highly differentiated cells in culture), they have nevertheless been plagued by poor efficiency (often far less than 0.1%), irreproducibility, and limited extensibility across different target host cell types. Further, the great majority of iPSCs used for disease mechanism studies (˜96%) are still generated by retroviral/lentiviral reprogramming methods. Bellin et al., Nat Rev Mol Cell Biol 13:713-726 (2012). While certain non-integrating reprogramming methods (e.g., Adenovirus, Sendai virus, episomal, mRNA, mature microRNA, and direct protein methods) do exist, these methods are so much less efficient than retro/lentiviral methods that their widespread application has been severely hampered.

Given the eventual therapeutic goal of generating patient-specific, immunocompatible biological material, there is a great need in the art to establish a robust and reproducible means for reprogramming cells that avoids use of viral components, while providing effective reprogramming in significant quantities. Such improved methods would ideally possess high efficiency of reprogramming, consistent reproducibility, and be readily extendible to a variety of cell types.

### SUMMARY

In one aspect of the invention described herein provides a method to generate induced human pluripotent stem cells comprising delivering to a somatic or non-embryonic cell population an effective amount of one or more reprogramming factors and also an agent that downmodulates SIRT2, and culturing the somatic or non-embryonic cell population for a period of time sufficient to generate at least one induced human pluripotent stem cell. In one embodiment of any aspect, the method further comprises delivering to the somatic or non-embryonic cell population an effect amount of an agent that upmodulates SIRT1. Exemplary agents that upmodulate SIRT1 include, but are not limited to, a small molecule, a peptide, or an expression vector encoding SIRT1.

In one embodiment of any aspect, the agent that downmodulates SIRT2 is a small molecule, an antibody, a peptide, an antisense oligo, or an inhibitory RNA (RNAi). Exemplary RNAi include, but are not limited to, microRNA, siRNA, or shRNA. In one embodiment of any aspect, the microRNA is a miR-200c-5p.

In one embodiment of any aspect, the method further comprises delivering to the cells one or more microRNAs selected from the miR-302/367 cluster.

In one embodiment of any aspect, the at least one reprogramming factor is an agent that increases the expression of c-Myc, Oct4, Nanog, Lin-28, or Klf4 in the cells. In another embodiment of any aspect, the reprogramming factor is an agent that increases the expression of SV40 Large T Antigen (“SV40LT”), or a short hairpin targeting p53 (“shRNA-p53”).

In one embodiment of any aspect, delivery comprises contacting the cell population with an agent, or a vector that encodes the agent. Delivery can comprise transduction, nucleofection, electroporation, direct injection, and/or transfection.

In one embodiment or any aspect, the vector is not-integrative or integrative. Exemplary non-integrative vectors include, but are not limited to, an episomal vector, EBNA1, a minicircle vector, a non-integrative adenovirus, non-integrative RNA, or a Sendai virus. Exemplary integrative vectors include, but are not limited to a retrovirus, a lentivirus, and a herpe simplex virus. In one embodiment or any aspect, the vector is a lentivirus vector.

In one embodiment or any aspect, the culturing is for a period of from 7 to 21 days.

In one embodiment or any aspect, SIRT2 is downmodulated by at least about 50%, 60%, 70%, 80% or 90% as compared to an appropriate control. In one embodiment or any aspect, SIRT1 is upmodulated by at least about 2×, 5×, 6×, 7×, 8×, 9×, or 10× as compared to an appropriate control. In one embodiment of any aspect, an appropriate control can be a cell population that an agent described herein has been delivered to.

In one embodiment of any aspect, the methods described herein result in at least a 2× enhancement of the number of induced pluripotent stem cells is produced as compared to an appropriate control.

One aspect of the invention described herein provides a cell line comprising induced pluripotent stem cells generated by any methods described herein.

One aspect of the invention described herein provides a pharmaceutical composition comprising an induced pluripotent stem cell or population thereof generated by any method described herein, and a pharmaceutically acceptable carrier.

Another aspect of the invention described herein provides a method to induce the differentiation of human pluripotent stem cells or cancer cells into differentiated somatic cells comprising exposure of said human pluripotent stem cells or cancer cells to a first agent that upregulates the expression or levels of SIRT2 combined with exposure to a second agent that downregulates the expression or levels of SIRT1.

Yet another aspect of the invention described herein provides a method to generate differentiated cells comprising delivering to a pluripotent cell population an agent that upmodulates SIRT2, and culturing the cell population under differentiating conditions for a period of time sufficient to generate at least one differentiated cell. In one embodiment, the method further comprises delivering to the pluripotent cell population an agent that downmodulates SIRT1.

In one embodiment of any aspect, the pluripotent cell population is an embryonic stem cell population, an adult stem cell population, an induced pluripotent stem cell population, or a cancer stem cell population.

In one embodiment of any aspect, the agent that downmodulates SIRT1 is a small molecule, an antibody, a peptide, an antisense oligonucleotide, or an RNAi.

In one embodiment of any aspect, the agent that upmodulates SIRT2 is selected from the group consisting of a small molecule, a peptide, and an expression vector encoding SIRT2.

In one embodiment of any aspect, the culturing is for a period of from 7 to 300 days.

In one embodiment of any aspect, SIRT1 is downmodulated by at least about 50%, 60%, 70%, 80% or 90% as compared to an appropriate control. In one embodiment of any aspect, SIRT2 is upmodulated by at least about 2×, 5×, 6×, 7×, 8×, 9×, or 10× as compared to an appropriate control.

In one embodiment of any aspect, the methods described herein result in at least a 2× enhancement of the number of differentiated cells is produced as compared to an appropriate control.

In one embodiment of any aspect, the differentiated cells are produced in a significantly shorter period of time as compared to an appropriate control.

In one embodiment of any aspect, the differentiating conditions are specific for neuronal differentiation to thereby generate neuronal cells.

One aspect of the invention described herein provides a cell line comprising differentiated cells generated by any of the methods described herein.

One aspect of the invention described herein provides a method to distinguish the status or fate of a cell or a cell population comprising measuring the levels and/or regulation of SIRT1 and SIRT2 in said cell or cell population. In one embodiment, a measurement of upregulated SIRT1 and downregulated SIRT2 distinguishes or defines a pluripotent stem cell status. In one embodiment, a measurement of downregulated SIRT1 and upregulated SIRT2 distinguishes or defines a somatic differentiated cell status.

Another aspect of the invention described herein provides a method from selecting pluripotent stem cells from an induced population comprising measuring the level and/or activity of SIRT1 and SIRT2 in a population of candidate cells, and selecting cells which exhibit an increased level and/or activity of SIRT1 and decreased level and/or activity of SIRT2. In one embodiment, the candidate cells are produced by any of the methods described herein.

Yet another aspect of the invention described herein provides a method for selecting differentiated cells from an induced population comprising measuring the level and/or activity of SIRT1 and SIRT2 in a population of candidate cells, and selecting cells which exhibit an increased level and/or activity of SIRT2 and decreased level and/or activity of SIRT1. In one embodiment, the candidate cells are differentiated by any of the methods described herein.

In one embodiment of any aspect, the measuring is by immunofluorescence.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” also refers to a subset of progenitors that have the capacity or potential, under particular circumstances, to differentiate to a more specialized or differentiated phenotype, and which retain the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent/pluripotent cell which itself is derived from a multipotent/pluripotent cell, and so on. While each of these cells may be considered stem cells, the range of cell types each can give rise to may vary considerably.

The term “pluripotent” as used herein refers to a cell with the capacity, under appropriate differentiation conditions, to differentiate into any type of cell in the body. Embryonic stem cells are considered ‘pluripotent’.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc. . . . ), but it cannot naturally form neurons. The term “multipotency” refers to a cell with the degree of developmental versatility that is less than totipotent and pluripotent.

The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue.

The term “differentiated cell” refers to a cell of a more specialized cell type derived from a cell of a less specialized cell type (e.g., a stem cell such as an induced pluripotent stem cell) in a cellular differentiation process. In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

It is possible that due to experimental manipulation cells that begin as stem cells might proceed toward a differentiated phenotype, but then (e.g., due to manipulation such as by the methods described herein) “reverse” and re-express the stem cell phenotype. This reversal is often referred to as “dedifferentiation” or “reprogramming” or “retrodifferentiation”. Similarly, cells that are de-differentiated to become multipotent or pluripotent can then be differentiated into a different differentiated phenotype.

As used herein, the term “adult cell” refers to a cell found throughout the body after embryonic development.

As used herein, a “somatic cell” refers to a cell that is not a germ line cell. A somatic cell can be a fibroblast derived from various organs or tissues, e.g., dermus, cardiac tissue, lung tissue, or the periodontal ligament.

The cells used in the methods and compositions described herein may be derived from any subject. The term “subject” as used herein refers to human and non-human animals. The term “non-human animals” and includes all vertebrates, e.g., mammals, such as non-human primates, (particularly higher primates), sheep, dog, rodent (e.g. mouse or rat, guinea pig), goat, pig, cat, rabbits, cows, and non-mammals such as chickens, amphibians, reptiles etc. In one embodiment, the subject is human. In another embodiment, the subject is an experimental animal or animal substitute as a disease model.

As used herein, “culturing” refers to maintaining a cell population in conditions (e.g., type of culture medium, nutrient composition of culture medium, temperature, pH, O₂ and/or CO₂ percentage, humidity level) suitable for growth.

As used herein, an “appropriate control” refers to an untreated, otherwise identical cell or population (e.g., a stem cell population or differentiated cell population that was not contacted by an agent described herein, or was contacted by only a subset of agents described herein, as compared to a non-control cell).

As used herein, “reprogramming factors” refers to factors used to dedifferentiate a cell population. A number of such factors are known in the art, for example, a set of transcription factors that have been identified to, e.g., promoting dedifferentiation. Exemplary reprogramming factors include, but are not limited to Oct3, Sox1, Sox2, Sox3, Sox15, Klf1, Klf2, Klf4, Klf5, c-Myc, L-Myc, N-Myc, Nanog, Lin-28, SV40LT, Glis1, and p53 shRNA. In one embodiment, a reprogramming factor is an environmental condition, such as serum starvation.

The term “downmodulate”, “decrease”, “reduce”, or “inhibit” are all used herein to mean a decrease by a reproducible statistically significant amount. In some embodiments, “downmodulate”, “decrease”, “reduce” or “inhibit” typically means a decrease by at least 10% as compared to a reference level (e.g. the absence of a given treatment) and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, as well as a 100% decrease.

The terms “upmodulate”, “increase”, “enhance”, or “activate” are all used herein to mean an increase by a reproducible statistically significant amount. In some embodiments, the terms “upmodulate”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to a reference level. In the context of a marker, an “increase” is a reproducible statistically significant increase in such level.

As used herein, “Sirtuin 1 (SIRT1)” refers to a NAD (nicotinamide adenine dinucleotide)-dependent deacetylase enzyme that regulates proteins essential for cellular regulation, e.g., via deacetylation. SIRT1 sequences are known for a number of species, e.g., human SIRT1, also known as SIRrL1 and SIR2alpha, (NCBI Gene ID: 23411) polypeptide (e.g., NCBI Ref Seq NP_001135970.1) and mRNA (e.g., NCBI Ref Seq NM_001142498.1). SIRT1 can refer to human SIRT1, including naturally occurring variants, molecules, and alleles thereof. SIRT1 refers to the mammalian SIRT1 of, e.g., mouse, rat, rabbit, dog, cat, cow, horse, pig, and the like.

As used herein, “Sirtuin 2 (SIRT2)” refers to a NAD-dependent deacetylase enzyme that functions as an intracellular regulatory protein with mono-ADP-ribosyltransferase activity. Among other roles, cytosolic SIRT2 has been shown to regulate processes such as microtubule acetylation and myelination, and nuclear SIRT2 facilitates methylation via deacetylation of H4K16. SIRT2 sequences are known for a number of species, e.g., human SIRT2, also known as SIR2, SIR2L, and SIR2L2, (NCBI Gene ID: 22933) polypeptide (e.g., NCBI Ref Seq NP 001180215.1) and mRNA (e.g., NCBI Ref Seq NM_001193286.1). SIRT2 can refer to human SIRT2, including naturally occurring variants, molecules, and alleles thereof. SIRT2 refers to the mammalian SIRT2 of, e.g., mouse, rat, rabbit, dog, cat, cow, horse, pig, and the like.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are envisioned. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA.

As used herein, the terms “protein” and “polypeptide” are used interchangeably herein to refer to a polymer of amino acids. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The term “short interfering RNA” (siRNA), also referred to as “small interfering RNA” is defined as an agent which functions to inhibit expression of a target gene, for example SIRT1 or SIRT2, e.g., by RNAi. As used herein an “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). An siRNA can contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA). An siRNA can be chemically synthesized, it can be produced by in vitro transcription, or it can be produced within a host cell.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs function as RNAi and/or siRNA species but differs in that shRNA species are double stranded hairpin-like structure for increased stability. These shRNAs can be contained in plasmids, retroviruses, or non-retroviruses such as lentiviruses and expressed from, for example, the pol III U6 promoter, or another promoter (see, e.g., Stewart, et al. (2003) RNA April; 9(4):493-501, incorporated by reference herein in its entirety).

The terms “microRNA” or “miRNA” are used interchangeably and these are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

As used herein, the term “expression vector” refers to a vector that directs expression of an RNA or polypeptide (e.g., a polypeptide encoding SIRT1) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing.

A vector can be integrating or non-integrating. “Integrating vectors” have their delivered RNA/DNA permanently incorporated into the host cell chromosomes. “Non-integrating vectors” remain episomal which means the nucleic acid contained therein is never integrated into the host cell chromosomes. Examples of integrating vectors include retrovirual vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The cells generated by the herein methods can be in a composition comprising a pharmaceutically acceptable carrier. The term “pharmaceutically acceptable carrier” as used herein means a pharmaceutically acceptable material, composition or vehicle, such as a liquid or solid filler, diluent, excipient, solvent or encapsulating material, involved in carrying or transporting the active ingredient (e.g., cells) to the targeting place in the body of a subject. Each carrier must be “acceptable” in the sense of being compatible with the other ingredients of the formulation and is compatible with administration to a subject, for example a human. In one embodiment, the carrier is something other than water or cell culture media.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1I present results from experiments that indicate SIRT2 downregulation and SIRT1 upregulation is a molecular signature of human pluripotency. (FIG. 1A) Immunoprecipitation of hDF and hESCs proteins using antibodies against acetyl-Lys, following LC-MS/MS analyses to identify acetylated proteins. (FIG. 1B) Mean value scatter plot of relative expression levels of SIRT1 and SIRT2 in hESC lines (n=25) and normal somatic cell lines (n=15) using results from a database search (which can be found on the world wide web at http://www.nextbio.com). All cell line information is shown in Table 6. (Mean±s.e.m., two-tailed unpaired Student's t-test.) (FIG. 1C) SIRT1 and SIRT2 expression from hDFs, iPSCs and hESCs was determined by qRT-PCR. (Mean±s.e.m., n=3 biologically independent experiments, * P<0.05; ** P<0.01; ***P<0.005, one-way ANOVA with Newman-Keuls post-test.) (FIG. 1D) Protein levels of SIRT1 and SIRT2. (FIG. 1E) Relative mRNA levels of SIRT1, SIRT2, Oct4 and SOX2 during in vitro differentiation of hESCs. (n=2 biologically independent experiments.) (FIG. 1F) Immunofluorescence assays of pluripotency markers (Oct4 and Tra-1-60) and neuronal markers (TH and Tuj1) before and after in vitro DA differentiation, respectively. Hoechst was used to show nucleus. Scale bar, 100 Gm. (FIGS. 1G and 1H) Gene expression levels of DA neuronal markers (TH, Lmx1b, and Tuj1) (FIG. 1G) and pluripotency markers (FIG. 1H) are shown along with those of SIRT1 and SIRT2. (Mean±s.e.m., n=3 biologically independent experiments, * P<0.05; ***P<0.005, two-tailed unpaired Student's t-test.) (FIG. 1I) SIRT1 and SIRT2 protein levels during in vitro DA differentiation.

FIGS. 2A-2G present results from experiments that indicate SIRT2 regulates acetylation and enzymatic activity of glycolytic enzymes. (FIG. 2A) Left: representative pictures of inducible SIRT2-GFP H9 hESCs with or without doxycycline (Dox). Scale bar, 100 iun. Right: the efficiency of SIRT2 overexpression was confirmed by western blotting with SIRT2-specific antibody. (FIGS. 2B-2D) Total protein extracts from wild-type (mock) and inducible SIRT2-GFP hESCs (SIRT2OE) with or without Dox were immunoprecipitated with anti-Aldolase A, anti-PGKI, anti-Enolase or anti-GAPDH antibodies (FIG. 2B) or anti-acetyl-Lys (FIG. 2C). Acetylation levels of each enzyme were assessed by western blotting with an anti-acetyl-Lys antibody (FIG. 2B) or each specific antibody (FIG. 2C). Enzymatic activities in each extracts are shown in FIG. 2D. Western blotting of Aldolase A, PGK1, Enolase, GAPDH, and β-actin using equal amounts of extracts are shown as the control (input). (Mean±s.d., n-=3 biologically independent experiments, *** P<0.005, two-way ANOVA with Bonferroni post-test). (FIG. 2E) Total proteins from mock and SIRT20E with or without Dox were immunoprecipitated using anti-Aldolase A or anti-Enolase antibodies and western blotting was performed with anti-acetyl-Lys or anti-SIRT2 antibodies. Aldolase A, Enolase, and β-actin western blotting of whole cell lysate (input) form wild-type and SIRT2-GFP hESCs were used as control of equal protein concentration for the IP. (FIGS. 2F and 2G) Total protein extracts from mock and SIRT2 knockdown (KD) hDFs were immunoprecipitated by anti-Aldolase A, anti-PGK1, anti-Enolase or anti-GAPDH antibodies. Acetylation levels and enzyme activity of Aldolase A, PGK1, Enolase, or GAPDH were determined by westernblotting with anti-acetyl-Lys antibody (FIG. 2F) and enzymatic assays (FIG. 2G), respectively. Aldolase A, PGK1, Enolase, GAPDH, and b-actin western blotting of whole cell lysates (input) from WT and SIRT2KD hDFs were used as control of equal concentration for the IP and enzymatic activity assays. (Mean±s.d. shown. n=3 biologically independent experiments, *P<0.05, two-way ANOVA with Bonferroni post-test.)

FIGS. 3A-3F results from experiments that indicate acetylation status of K322 regulates AldoA activity. (FIG. 3A) Western blotting shows that AldoA-Myc is highly acetylated in SIRT2KD 293T cells although total proteins are unchanged. (FIG. 3B) Sequence alignment of putative acetylation sites (K111 and K322) from different species. (FIG. 3C) Myc-tagged AldoA, AldoAKI 11Q, and AldoAK322Q were each expressed in hDFs. AldoA proteins were purified by IP with a Myc antibody, and specific activity for AldoA was determined. (Mean±s.d., n=3 biologically independent experiments, ***P<0.005, one-way ANOVA with Bonferroni post-test.) (FIG. 3D) Myc-tagged AldoA, AldoAK111R, and AldoAK322R were each expressed in hDFs co-expressing SIRT2 shRNA (SIRT2KD). AldoA proteins were purified by IP with Myc antibody, and specific activity for AldoA was determined. (Mean±s.d, n=3 biologically independent experiments, ***P<0.005, one-way ANOVA with Bonferroni post-test.) (FIG. 3E) Crystal structure model of human AldoA (Protein Data Bank code: 1ALD). (FIG. 3F) Identified acetylated Lys in indicated sample.

FIGS. 4A-4H present results from experiments that indicate SIRT2 influences metabolism and cell survival of hPSCs. (FIG. 4A) Glycolytic bioenergetics of wild-type (mock) and inducible SIRT2-GFP H9 hESCs (SIRT2OE) with or without Dox were assessed using the Seahorse XF analyzer. Mean±s.d. shown. n=3 biologically independent experiments. (FIG. 4B) Basal glycolytic rate, glycolytic capacity and glycolytic reserve from mock and SIRT2OE with or without Dox shown in FIG. 4A. (Mean±s.d., n=3 biologically independent experiments, *P<0.05, one-way ANOVA with Bonferroni posttest.) (FIG. 4C) Cell proliferation of mock and SIRT2OE H9 hESCs with or without Dox was analyzed by determining cell numbers every two days under ESC culture condition. (Mean±s.d., n=3 biologically independent experiments, ***P<0.005, two-way ANOVA with Bonferroni post-test.) (FIG. 4D) GFP-positive (GFP⁺) WT and SIRT2 H9 hESCs with or without Dox were mixed at a ratio of 1:1 with GFP-negative (GFP⁻) hESCs, respectively. The GFP⁺/GFP⁻ ratios were measured at each passage. (Mean±s.d., n=3 biologically independent experiments, ***P<0.005, two-way ANOVA with Bonferroni post-test.) (FIG. 4E) Apoptotic population of mock and SIRT2OE H9 hESCs with or without Dox for three days under ESC culture conditions measured by Annexin V/7-AAD staining. (FIG. 4F) Quantification of Annexin V positive cells in mock and SIRT2OE hESC lines (H9 and H7) and two iPSC lines (iPSC-1 and iPSC-2) with or without Dox. 1: Mock w/o Dox, 2: Mock with Dox, 3: SIRT2OE w/o Dox, 4: SIRT2OE with Dox. (Mean±s.d., n=3 biologically independent experiments, ***P<0.005, one-way ANOVA with Bonferroni post-test.) (FIG. 4G) Intracellular ROS levels of mock and SIRT2OE hPSCs (H9 and hiPSC-1) with or without Dox. 1: Mock w/o Dox, 2: Mock with Dox, 3: SIRT2OE w/o Dox, 4: SIRT2OE with Dox. (Mean±s.d., n=5 biologically independent experiments, ***P<0.005, one-way ANOVA with Bonferroni post-test.) (FIG. 4H) Effect of antioxidant on cell death of hPSCs (H9 and hiPSC-1) by SIRT2OE with or without Dox. 1: Veh only, 2: NAC, 3: Dox+Veh, 4: Dox+NAC. (Mean±s.d., n=3 biologically independent experiments, ***P<0.005, one-way ANOVA with Bonferroni posttest).

FIGS. 5A-5G present results from experiments that indicate SIRT2 influences metabolism during early in vitro differentiation of hESCs. (FIGS. 5A and 5B) Inducible SIRT2OE 1-19 hESCs were induced to differentiate spontaneously by culturing in serum-free 1TSFn medium for up to 4 days, and gene expression levels of pluripotency markers (Oct4 Nanog, and Rex1) (FIG. 5A) and early-differentiation markers (Pax6, Brachyury (B-T), and Sox17) (FIG. 5B) were determined by qRT-PCR. (Mean±s.d., n=3 biologically independent experiments, *P<0.05; **P<0.01, one-way ANOVA with Bonferroni posttest.) (FIG. 5C) Expression level of SIRT2 in SIRT2OE 1-1H9 hESCs with or without Dox during early differentiation. (Mean±s.d., n==3 biologically independent experiments, *P<0.05, one-way ANOVA with Bonferroni posttest.) (FIG. 5D) Glycolytic bioenergetics of mock and SIRT2OE H9 hESCs with or without Dox were assessed using the Seahorse XF analyzer, (Mean±s.d., n=3 biologically independent experiments, *P<0.05, one-way ANOVA with Bonferroni post-test.) (FIG. 5E) Extracellular lactate production of mock and SIRT20E H9 hESCs with or without Dox. (Mean±s.d., nt=3 biologically independent experiments, *P<0.05; **P<0.01; ***P<0.005, one-way ANOVA with Bonferroni post-test.) (FIG. 5F) SIRT2OE H9 hESCs were induced to differentiate spontaneously for 7 days, and differentiating cells were immunostained for the presence of lineage-specific markers for ectoderm (Otx2), endoderm (Sox17), and mesoderm (B-T). Scale bar, 100 um. (FIG. 5G) Heatmaps depicting gene expression levels of markers representing ectoderm (Pax6, Map2, GFAP and AADC), endoderm (Foxa2, Sox17, AFP, CK8 and CK18), and mesoderm (Msxl and B-T) in wild-type (Mock) and inducible SIRT2-GFP (SIRT2OE) H9 and H7 hESC lines with or without Dox differentiated for up to 12 days under differentiation condition. 1: Mock w/o Dox, 2: Mock with Dox, 3: SIRT2OE w/o Dox, 4: SIRT2OE with Dox. (n=2 biologically independent experiments).

FIGS. 6A-6K present results from experiments that indicate SIRT2KD facilitates metabolic reprogramming in fibroblasts during the induced pluripotency. (FIGS. 6A and 6B) Oxygen consumption rate (OCR) (FIG. 6A) and ECAR (FIG. 6B) of human fibroblasts (hDFs) infected with control (siNS) or SIRT2 siRNA (siSTRT2) at 3 days after transfection were assessed by XF analyser. (Mean±s.d., n=3 biologically independent experiments, *P<0.05, two-tailed unpaired Student's t-test.) (FIG. 6C) OXPHOS capacity of hDFs infected with siNS or siSIRT2 at 3 days after transfection. (Mean±s.d., n=3 biologically independent experiments.) (FIGS. 6D and 6E) Basal respiration. ATP turnover, maximum respiration. oxidative reserve (FIG. 6D) or relative OCR changes after FCCP injection (FIG. 6E) from siNS and siSIRT2 are shown in c. (Mean±s.d., n=3 biologically independent experiments, **P<: 0.01; ***P<0.005, two-tailed unpaired Student's t-test.) (FIGS. 6F and 6G) OCR were shown for hDFs infected with lentiviruses expressing four reprogramming factors (Y4) and/or SIRT2 knockdown (SIRT2KD) at 3 (FIG. 6F) or 8 (FIG. 6G) days after transfection. (Mean±s.d., n=3 biologically independent experiments.) (FIGS. 6H and 6I) Basal respiration, ATP turnover, maximum respiration, and oxidative reserve from Y4 and/or SIRT2KD at 3 (FIG. 6H) or 8 (FIG. 6I) days after transfection are shown in FIGS. 6F and 6G (Mean±s.d., n=3 biologically independent experiments. * P<0.05; ** P<0.01; ***P<0.005, one-way ANOVA with Bonferroni posttest.) (FIGS. 6J and 6K) OCR/ECAR ratio (FIG. 6J) or relative OCR changes after FCCP injection (FIG. 6K) from Y4 and/or SIRT2KD are shown in f,g. (Mean±s.d., n==3 biologically independent experiments, * P<0.05; ** P<0.01; ***P<0.005, one-way AN OVA with Bonferroni post-test).

FIGS. 7A-7I present results from experiments that indicate SIRT2 influences somatic nuclear reprogramming through metabolic changes. (FIG. 7A) Time course of expression level of SIRT2 mRNA in hDFs infected with Y4 and/or SIRT2KD. (Mean±s.d., n=4 biologically independent experiments, **P<0.01; ***P<0.005, two-way ANOVA with Bonferroni post-test.) (FIGS. 7B and 7C) OCR (FIG. 7B) and ECAR (FIG. 7C) in hDFs infected with Y4 and/or SIRT2KD were assessed by XF analyzer. (Mean±s.d., n=4 biologically independent experiments, *P<0.05; **P<0.01; ***P<0.005, two-way ANOVA with Bonferroni post-test.) (FIG. 7D) Measurement of lactate production from hDFs infected with Y4 and/or SIRT2KD. (Mean±s.d., n=3 biologically independent experiments, ***P<0.005, two-way ANOVA with Bonferroni post-test.) (FIGS. 7E and 7F) Effects of SIRT2OE or KD on iPSC generation. Upper: The efficiency of overexpression (FIG. 7E) or knockdown (FIG. 7F) was confirmed by western blotting with anti-SIRT2 antibody. Lower: Representative pictures of AP-positive colonies at 14 days post-infection (dpi). (Mean±s.e.m., n=3 biologically independent experiments, **P<0.01, two-way ANOVA with Bonferroni post-test.) (FIGS. 7G and 7H) Effects of glycolytic inhibitor, 2-deoxyglucose (2DG) on iPSC generation by Y4 and/or STRT2KD at 8 days post-transduction were assessed by OCR (FIG. 7G) and ECAR (FIG. 7H). (Mean±s.d., n=4 biologically independent experiments, **P<0.01; ***P<0.005, two-way ANOVA with Bonferroni post-test.) (FIG. 7I) Effects of 2DG on iPSC generation by Y4 and/or SII*2KD. Representative pictures of AP-positive colonies at 14 days post-transduction. (Mean±s.d., n=3 biologically independent experiments, ***P<0.005, two-way ANOVA with Bonferroni post-test.)

FIGS. 8A-8G present results from experiments that indicate miR-200c directly targets SIRT2. (FIGS. 8A and 8B) Altered expression levels of SIRT2 by pre-miRNAs were analysed by qRT-PCR (FIG. 8A) or western blotting with SIRT2-specific antibody (FIG. 8B). (Mean±s.d., n=3 biologically independent experiments, **P<0.01, one-way ANOVA with Bonferroni posttest.) (FIG. 8C) Sequences for stem loop of miR-200c (upper) and matured forms of miR-200c-5p and -3p (lower). (FIGS. 8D and 8E) Altered expression levels of SIRT2 by miRNA mimics for control (Scr), miR-200c-5p (5p) and -3p (3p) were analysed by qRT-PCR (FIG. 8D) or western blotting with SIRT2-specific antibody (FIG. 8E). (Mean±s.d., n=3 biologically independent experiments, ***P<0.005, one-way ANOVA with Bonferroni post-test.) (FIG. 8F) Luciferase validation assays demonstrating the effect of miR-200c-5p on the CDS fragments of SIRT2 relative to control (Scr) in 293T cells. (Mean±s.d., n=3 biologically independent experiments, **P<0.01, one-way ANOVA with Bonferroni post-test.) (FIG. 8G) Proposed model for miR-200c-SIRT2-glycolytic enzymes (aldolase, GAPDH, enolase, and PGK1) axis in regulating metabolic switch and somatic reprogramming.

FIG. 9 presents results from experiments that indicate combined effects of SIRT1 overexpression (OE) and SIRT2 knock-down (KD) on human iPSC generation. Fibroblasts were treated with lentiviruses expressing four reprogramming factors with or without SIRT1OE or SIRT2KD. Representative pictures of AP-positive colonies at day 14 post lentiviral transduction. Mean±s.d., n=3 biologically independent experiments, *** P<0.005, two-way ANOVA with Bonferroni post-test.

FIG. 10 presents results from experiments that indicate SIRT1 expression is variable in cancer. Although some cancer cells appear to express higher levels of SIRT1, it is not consistent like ESCs and iPSCs. It is however expected that SIRT1 is consistently highly expressed in cancer stem cells.

FIG. 11 presents results from experiments that indicate SIRT2 expression is variable in cancer. Although some cancer cells appear to express lower levels of SIRT2, it is not consistent like ESCs and iPSCs. It is expected that SIRT2 is consistently down-regulated in cancer stem cells.

FIGS. 12A-12G present results from experiments that indicate Warburg-like effect in hESCs and hiPSCs compared to hDFs. (FIG. 12A) Human ESCs (H9) and hiPSCs cultured under feeder-free condition were stained with specific antibodies against pluripotency markers (e.g., Oct4, Nanog, SSEA4, and TRA1-60) along with Hoechst staining for nuclear staining. Scale bar=100 pm. (FIG. 12B) Representative pictures of hESCs and hiPSCs. (FIG. 12C) In vitro spontaneous differentiation of hESCs and hiPSCs by culturing in serum-free ITSFn medium for 7 days. Immunostaining images (first and second row panels) show lineage specific markers for ectoderm (0tx2), mesoderm (Brachyury; B-T), and endoderm (Sox17). Scale bar=: 100 pm. (FIG. 12D) Intracellular ATP levels were significantly lower in hiPSCs and hESCs than in the original fibroblasts. Mean±SEM (n=3) are shown. ***p<0.005. (FIG. 12E) Mitochondria bioenergetics of parental hDFs and hiPSCs as well as hESCs assessed by Seahorse XF analyzer. (FIG. 12F) Expression levels of glucose transporters (GLUTs) including GLUT1 to GLUT7 in hDFs and hiPSCs as well as hESCs. Mean±SEM (n=3) are shown. * p<0.5; ** p<0.01; ***p<0.005; ****p<0.001. (FIG. 12G) Immunoprecipitation of hDF and hESCs proteins using antibodies against acetyl-Lys, followed by LC-MS/MS analyses to identify acetylated proteins.

FIG. 13 presents results from experiments that indicate CID spectra for the acetylated proteins shown in FIG. 12 and Table 2. Peptides for tubulin, Fructose-biphosphate aldolase, glyceraldehyde-3-phosphate dehydrogenase, phosphoglycerate kinase 1, enolase, pyruvate kinase isozymes M1/M2 and ATP synthase were detected via combination of IP and LC-MS/MS analysis. IP was performed with anti-acetyl-Lys antibody.

FIGS. 14A-14G present results from experiments that indicate meta-analysis of Sirtuin family expression in hESCs. (FIG. 14A) Compiled data used in this study for Sirtuin family gene expression in hESCs shown in Table 5. Expression levels of each Sirtuin shown as up, down, and N/A, which corresponds to upregulated, downregulated, and no significant change, respectively. Numbers in parenthesis represent expression changes from 5 different studies. (FIG. 14B) Representative data showing SIRT2 expression changes between different cells. SIRT2 downregulation was observed in hPSCs compared to differentiated cells and original fibroblasts. (FIGS. 14C-14G) Expression levels comparison of SIRT3 (FIG. 14C), SIRT4 (FIG. 14D), SIRT5, (FIG. 14E) SIRT6 (FIG. 14F), and SIRT7 (FIG. 14G), across several hESC lines and normal non-cancer cell lines based on Database analyses (found on the world wide web at http://www.nextbio.com). The relative expression levels are presented as the mean value of scatter plot.

FIGS. 15A-15D present results from experiments that indicate characterization of inducible SIRT2-GFP H9 hESCs. (FIG. 15A) Plasmid map of the EGFP SIRT2 doxycycline (Dox) inducible overexpression vector. (FIG. 15B) Expression levels of glycolytic enzymes in SIRT2-GFP hESCs with or without Dox analyzed by qRT-PCR. Mean±SEM (n=3) are shown. * p<0.005. (FIGS. 15C and 15D) Expression levels of pluripotency markers in hESCs, hDFs, and SIRT2-GFP hESCs with or without Dox. Mean±SEM (n=3) are shown. *p<0.005.

FIGS. 16A-16F present results from experiments that indicate effects of altered SIRT2 expression on acetylation of AldoA. (FIGS. 16A-16D) Detection of AldoA K111 (FIGS. 16A and 16B) and K322 (FIGS. 16C and 16D) acetylation by mass spectrometry analysis. Symbol “@” indicates the acetylation site. (FIG. 16E) Myc-tagged AIdoA, AldoAK111Q, and AldoAK3224 were each expressed in 293T cells. AldoA proteins were purified by IP with Myc antibody, and specific activity for AldoA was determined. MeantSEM (n===3) are shown. *0* p<0.005. (FIG. 16F) Myc-tagged AldoA, AldoAK111R, and AldoAK322R were each expressed in 293T cells co-expressing SIRT2 shRNA (SIRT2KD). AldoA proteins were purified by IP with Myc antibody and specific activity for AldoA was determined. MeantSEM (n=3) are shown. ***p<0.005.

FIGS. 17A-17H present results from experiments that indicate metabolic and functional characterization of hPSC lines following SIRT2 overexpression. (FIGS. 17A, 17C, and 17E) Glycolytic bioenergetics of wild type (Mock) and inducible SIRT2-GFP cell lines from H7 hESCs (FIG. 17A) and two iPSC lines (FIGS. 17C and 17E) with or without Dox were assessed by XF analyzer. (FIGS. 17B, 17D, and 17F) Basal glycolytic rate, glycolytic capacity and glycolytic reserve of mock and SIRT2OE from H7 hESCs (FIG. 17B) and two iPSC lines (FIGS. 17D and 17F) with or without Dox are shown in FIGS. 17A, 17C, and 17E, respectively. Mean±SEM (n=3) are shown. *p<0.05; ″p<0.01. (FIG. 17G) OCR were shown for two hESC lines (H9 and H7) and hiPSC-1 line with or without Dox. 1: Mock w/o Dox, 2: Mock with Dox, 3: SIRT2OE w/o Dox, 4: SIRT2OE with Dox. Mean SEM (n=3) are shown. *p<0.05; ***p<0.005 (FIG. 17H) Cell proliferation of mock and SLRT2OE from H7 hESCs and two independent iPSC lines (hiPSC-1 and hiPSC-2) with or without Dox were analyzed by determining cell numbers every 2 days under ESC culture conditions. Mean±SEM (n=3) are shown. ″p<0.01; ***p<0.005.

FIGS. 18A-18F present results from experiments that indicate SIRT2 influences metabolic signatures of early differentiation potential of hiPSCs. (FIGS. 18A and 18B) Inducible SIRT2OE hiPSC-1 cells were induced to differentiate spontaneously by culturing serum-free ITSFn medium for up to 4 days, and gene expression levels of pluripotency markers (Oct4. Nanog, and Rex1) (FIG. 18A) and early-differentiation markers (Pax6, Brachyury (B-T), and Sox17) (FIG. 188) were determined by qRT-PCR. Mean±SEM (n=3) are shown. * p<0.05; ** p<0.01. (FIG. 18C) Expression level of SIRT2 in SIRT2OE hiPSC-1 cells with or without Dox during early differentiation. Mean±SEM (n=3) are shown * p<0.05. (FIG. 18D) Glycolytic bioenergetics of mock and SIRT2OE hiPSC-1 cells with or without Dox were assessed using the Seahorse XF analyzer. Mean±SEM (n=3) are shown. * p<0.05. (FIG. 18E) Extracellular lactate production of mock and SIRT2OE hiPSC-1 cells with or without Dox. Mean±SEM (n=3) are shown. * p<0.05; ** p<0,01. (FIG. 18F) Heatmaps depicting gene expression levels of markers representing ectoderm (Pax6, Map2, GFAP and AADC), endoderm (Foxa2, Sox17, AFP, CK8 and CK18), and mesoderm (Msx1 and B-T) in wild type (Mock) and inducible SIRT2OE hiPSC lines including hiPSC-1 and hiPSC-2 with or without Dox for up to 12 days under differentiation condition. Mean±SEM (n=3) are shown. 1: Mock w/o Dox, 2: Mock with Dox, 3: SIRT2OE w/o Dox. 4: SIRT2OE with Dox.

FIGS. 19A-19H present results from experiments that indicate effects of altered SIRT1 expression on metabolic reprogramming and iPSC generation. (FIG. 19A) Plasmid map of the EGFP SIRT1 doxycycline inducible overexpression vector. (FIG. 19B) OCR was shown for hDFs infected with wild type (Mock) or inducible SIRT1-GFP (SIRT1OE) with or without Dox at 3 days after transfection. (FIGS. 19C and 19D) OCR/ECAR ratio (FIG. 19C), and relative OCR changes after FCCP injection (FIG. 19D) from Mock and SIRT1OE with or without Dox are shown in FIG. 19B, Mean±SEM (n=3) are shown. (FIGS. 19E and 19F) Effects of SIRT1KD or OE on iPSC generation. Upper: Efficiency of SIRT1KD or OE was confirmed by western blotting with anti-SIRT1 antibody. Lower: Representative pictures of AP-positive colonies at day 14 post lentiviral transduction. Mean±SEM (n=3) are shown. *p<0.005 G,H: OCR in hDF infected with Y4 and/or SIRT1 OE at 3 (FIG. 19G) or 6 (FIG. 19H) days after transfection.

### DETAILED DESCRIPTION

Aspects of the invention are based on the discovery that the metabolic pathway used by a cell directly influences its state of differentiation. Although correlations between cellular metabolism and differentiation state have been previously observed, a causative effect of metabolism on cell state was not appreciated. The results presented herein indicate that the metabolic pathway utilized drives a cell either towards pluripotency or differentiation. As such metabolic reprogramming (e.g., via experimental manipulation) can directly influence the differentiated state of a cell. Reprogramming cells to increase utilization of glycolysis metabolism and decrease oxidative phosphorylation (OXPHOS) metabolism drives cells to a less differentiated state (to thereby increase their “stemness”). Whereas, reprogramming cells toward decrease utilization of glycolysis and increase OXPHOS metabolism drives cells towards a more differentiated state.

Aspects of the invention are further based on the finding that one way in which a cell regulates which metabolic pathway is utilized is through protein acetylation, with acetylated glycolytic enzymes being highly active compared to their deacetylated counterparts. This, taken with the recognition of the role of the different metabolic pathways in cell fate, indicates that cell fate can be manipulated by the appropriate manipulation of the acetylation state of glycolytic enzymes.

As such, one aspect of the invention relates to the shifting of cell fate by manipulation of the acetylation state of the glycolytic enzymes. Deacetylation of the glycolytic enzymes in otherwise differentiated cells (e.g., somatic cells) to thereby reduce glycolysis in the cells, shifts the cells towards pluripotency. Alternatively, acetylation of the glycolytic enzymes in less differentiated cells to thereby increase glycolysis in the cells (e.g., pluripotent or multipotent) shifts the cells towards differentiation.

One such method of reducing glycolysis is through manipulation of the deacetylase SIRT2. SIRT2 deacetylates glycolytic enzymes to thereby reduce their activity and suppress glycolysis. SIRT2 is highly active in differentiated cells. Reduction in SIRT2 activity allows glycolysis to increase thereby driving the cells toward de-differentiation. Alternatively, SIRT2 activity in less differentiated cells (e.g., stem cells) is relatively low, as is glycolytic enzyme activity, with OXPHOS being primarily used for metabolism. Increasing SIRT2 activity in less differentiated cells deacetylates the glycolytic enzymes, suppressing glycolysis, and drives the cells toward a more differentiated state.

Another acetylation modulating factor, SIRT1, has activity reciprocal to that of SIRT2 with respect to cell fate. SIRT1 is active in less differentiated cells, with activity decreasing in more differentiated cells. Similar to SIRT2, SIRT1 alters acetylation of metabolic enzymes to increase utilization of glycolysis and decrease utilization of OXPHOS, thereby contributing to the undifferentiated state. SIRT1 manipulation can therefore be used in the methods described herein to affect cell fate, with an increase in SIRT1 driving a cell towards de-differentiation and a decrease in SIRT1 driving a cell towards further differentiation.

The ability to shift cell fate by manipulating the metabolic pathways utilized is useful in enhancing known methods of cell fate manipulation (e.g. to generate pluripotent cells from differentiated cells, and to generate differentiated cells from pluripotent cells). Methods for de-differentiating cells using reprogramming factors are well known in the art. Examples include the induction of the Yamanaka (reprogramming) factors: Oct-4, Sox-2, c-Myc (or 1-Myc) and Klf-4, and also the induction of the Thomson (reprogramming) factors: Oct-4, Sox-2, Nanog, and Lin-28. Unfortunately, the current methods for inducing de-differentiation of a cell (e.g., pluripotency) are fairly inefficient, generating a small percentage of the desired product. Modulation of cell metabolism, such as by SIRT1 (upmodulation) and SIRT2 (downmodulation), as described herein, to shift a cell towards a less differentiated state can be used to enhance known methods for de-differentiating cells (e.g., generating induced pluripotent cells). As such, the methods involve SIRT1 and SIRT2 modulation in combination with the full complement of reprogramming factors. It is expected however, that SIRT1 and SIRT2 modulation, as described herein, will increase the number of de-differentiated cells produced and/or enable the omission of one or more of the reprogramming factors in the de-differentiation process. The ability to omit one or more reprogramming factors is considered an enhancement of the known procedures if it facilities a reduction in total manipulation of the cells (e.g., delivery of less foreign matter to the cells).

Various methods for differentiating cells (e.g., pluripotent or multipotent stem cells) by using various differentiation factors and/or culture procedures are known. Many of these methods suffer from low efficacy of induction and/or slow rate of induction. Modulation of cell metabolism, wuch as by SIRT1 (downmodulation) and SIRT2 (upmodulation), as described herein, to shift a cell toward a more differentiated state can be used to enhance known methods for differentiating cells (e.g., generating neuronal cells). As such, the methods involve SIRT1 and SIRT2 modulation in combination with known methods of differentiation. It is expected however, that SIRT1 and SIRT2 modulation will decrease the time required to generate the differentiated cells and/or increase the number of differentiated cells produced. It is also expected that SIRT1 and SIRT2 modulation will also enable the omission of one or more steps or factors required for the differentiation process.

Moreover, the invention described herein provides methods for selecting pluripotent stem cells and differentiated cells based on the expression level and/or activity of SIRT1 and/or SIRT2.

Methods and compositions described herein require that the levels and/or activity of SIRT1 and/or SIRT2 be modulated in order to more easily and readily alter the cell fate. SIRT1 is a NAD (nicotinamide adenine dinucleotide)-dependent deacetylase enzyme that regulates proteins essential for cellular regulation, e.g., via deacetylation. SIRT2 is a NAD-dependent deacetylase enzyme that functions as an intracellular regulatory protein with mono-ADP-ribosyltransferase activity.

Downmodulate or downmodulation refers to reducing the function of the protein (e.g., SIRT1 or SIRT2). This can be accomplished by directly inhibiting the production of functional SIRT1 or SIRT2 itself in the cell (e.g., by reducing gene expression or protein synthesis), or alternatively by reducing SIRT1 or SIRT2 function/activity. SIRT1 or SIRT2 function/activity can be reduced, for example by directly inhibiting the SIRT1 or SIRT2 protein itself or otherwise targeting that protein for degradation. As such, an agent useful in the present invention for downmodulation is one that inhibits SIRT1 or SIRT2 gene expression or protein synthesis, or inhibits SIRT1 or SIRT2 function or activity. Downmodulation of SIRT1 or SIRT2 can also be accomplished by inhibition of an upstream factor that induces or positively regulates SIRT1 or SIRT2 gene expression or SIRT1 or SIRT2 function/activity. As such, another useful agent for downmodulation is an agent that inhibits or downmodulates such an upstream factor by methods that correspond to those described for SIRT1 or SIRT2.

Upmodulate or upmodulation refers to increasing the level of a functional protein, and is accomplished by methods described for downmodulation, but by instead increasing or activating gene expression or protein activity.

Induced Pluripotent Stem Cells

Stem cells are undifferentiated cells defined by their ability at the single cell level to both self-renew and differentiate to produce progeny cells, including self-renewing progenitors, non-renewing progenitors, and terminally differentiated cells. Stem cells, depending on their level of differentiation, are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. “Induced pluripotent stem cells” are pluripotent stems cells that are generated directly from adult cells, e.g., somatic or non-embryonic cells.

One aspect of the invention described herein provides a method to generate induced human pluripotent stem cells comprising delivering to a somatic or non-embryonic cell population an effective amount of one or more reprogramming factors (e.g., Yamanaka factors or Thomson factors) and also an agent that downmodulates SIRT2, and culturing the somatic or non-embryonic cell population for a period of time sufficient to generate at least one induced human pluripotent stem cell. In one embodiment, the method further comprises delivering to the somatic or non-embryonic cell population an effective amount of an agent that upmodulates SIRT1.

In one embodiment, the somatic or non-embryonic cell population is cultured for a period of time sufficient to generate at least one induced human pluripotent stem cell. Culturing can occur for a period of at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, or more.

In some instances, the chemical and/or atmospheric conditions are altered for reprogramming. For example, where the somatic and non-embryonic cells are not vascularized and hypoxic reprogramming under hypoxic conditions of 5% O₂, instead of the atmospheric 21% O₂, may further provide an opportunity to increase the reprogramming efficiency. Similarly, chemical induction techniques have been used in combination with reprogramming, particularly histone deacetylase (HDAC) inhibitor molecule, valproic acid (VPA), which has been found wide use in different reprogramming studies.

At the same time, other small molecules such as MAPK kinase (MEK)-ERK (“MEK”) inhibitor PD0325901, transforming growth factor beta (“TGF-β”) type I receptor ALK4, ALK5 and ALK7 inhibitor SB431542 and the glycogen synthase kinase-3 (“GSK3”) inhibitor CHIR99021 have been applied for activation of differentiation-inducing pathways (e.g. BMP signaling), coupled with the modulation of other pathways (e.g. inhibition of the MAPK kinase (MEK)-ERK pathway) in order to sustain self-renewal. Other small molecules, such as Rho-associated coiled-coil-containing protein kinase (“ROCK”) inhibitors, such as Y-27632 and thiazovivin (“Tzv”) have been applied in order to promote survival and reduce vulnerability of cell death, particularly upon single-cell dissociation. As such, the inclusion of one or more of the factors in the herein described methods is envisioned.

Efficiency of Reprogramming

Efficiency of reprogramming, e.g., changing the cell fate of a cell, is readily ascertained by one of many techniques readily understood by the skilled practitioner. For example, efficiency can be described by the ratio between the number of donor cells receiving the agent(s) and reprogramming factors and the number of reprogrammed colonies (de-differentiated colonies) generated. The number donor cells receiving the agent(s) and reprogramming factors can be measured directly, such as by use of a reporter gene such as GFP included in a vector encoding an agent or reprogramming factor. Alternatively, indirect measurement of delivery efficiency can be accomplished by transfecting a vector encoding a reporter gene as a proxy to gauge delivery efficiency in paired samples delivering agent(s) and reprogramming factor vectors. Further, the number of reprogrammed colonies generated can be measured by, for example, observing the appearance of one or more multipotency or pluripotency characteristics such as alkaline phosphatase (AP)-positive clones, colonies with endogenous expression of transcription factors Oct-4 or Nanog, or antibody staining of surface markers such as Tra-1-60. Efficiency can alternatively be described by the time required for induced pluripotent stem cell generation. A combination of percentage of induced cells and the time of induction can also be used.

In one embodiment, the methods described herein result in an enhancement of the number of induced pluripotent stem cells by at least 2-fold as compared to an appropriate control. In another embodiment, the methods described herein result in an enhancement of the number of induced pluripotent stem cells by at least 3-fold, at least 4-fold, at least 5-fold, at least 6-fold, at least 7-fold, at least 8-fold, at least 9-fold, at least 10-fold, or more as compared to an appropriate control. As used herein, an “appropriate control” refers to a comparably treated cell population in the absence of the agent (e.g., that downmodulates SIRT2 and/or that upmodulates SIRT1). The efficiency of reprogramming can be assessed as described above.

One aspect of the invention described herein provides a cell line comprising induced stem-like cells (e.g., pluripotent stem cells) generated by any of the methods described herein.

Another aspect of the invention described herein provides a pharmaceutical composition comprising an induced stem-like cell (e.g., pluripotent stem cell) or population thereof generated by any of the methods described herein and a pharmaceutically acceptable carrier.

Reprogramming Factors with Downmodulation of SIRT2 and/or Upmodulation of SIRT1

The somatic or non-embryonic cell population is further contacted with one or more reprogramming factor. In one embodiment, the one or more reprogramming factor is from one to four reprogramming factors selected from the Yamanaka (reprogramming) factors, e.g, Oct-4, Sox-2, c-Myc (or 1-Myc) and Klf-4, or selected from the Thomson (reprogramming) factors, e.g., Oct-4, Sox-2, Nanog, and Lin-28. Reprogramming factors are traditionally understood to be normally expressed early during development and are involved in the maintenance of the pluripotent potential of a subset of cells that constitute the inner cell mass of the pre-implantation embryo and post-implantation embryo proper. Their ectopic expression is believed to allow the establishment of an embryonic-like transcriptional cascade that initiates and propagates an otherwise dormant endogenous core pluripotency program within a host cell.

In one embodiment, reprogramming factors are expressed in the cell e.g., via an vector such as those described herein, comprising a nucleic acid encoding a given reprogramming factor. In another embodiment, reprogramming factors are expressed in the cell e.g., via expression of a nucleic acid encoding a given reprogramming factor as naked DNA.

Additional reprogramming factors include, but are not limited to, Tert, Klf-4, c-Myc, SV40 Large T Antigen (“SV40LT”) and short hairpin RNAs targeting p53 (“shRNA-p53”). One or more of these factors can further be delivered to the cells to enhance the reprogramming process using delivery methods described herein.

The agent and reprogramming factors described herein may necessarily be contained in and thus further include a vector. Many such vectors useful for transferring exogenous genes into target mammalian cells are available. The vectors may be episomal, e.g. plasmids, virus-derived vectors (e.g., viral vectors) such cytomegalovirus, adenovirus, etc., or may be integrated into the target cell genome, through homologous recombination or random integration, e.g. retrovirus-derived vectors such as MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The use of various combinations of vectors in the methods is envisioned. While various vectors and reprogramming factors in the art appear to present multiple ingredients capable of establishing reprogramming in cells, a high degree of complexity occurs when taking into account the stoichiometric expression levels necessary for successful reprogramming to take hold. For example, somatic cell reprogramming efficiency is reportedly fourfold higher when Oct-4 and Sox-2 are encoded in a single transcript on a single vector in a 1:1 ratio, in contrast to delivering the two factors on separate vectors. The latter case results in a less controlled uptake ratio of the two factors, providing a negative impact on reprogramming efficiency. One approach towards addressing these obstacles is the use of polycistronic vectors, such as inclusion of an internal ribosome entry site (“IRES”), provided upstream of transgene(s) that is distal from the transcriptional promoter. This organization allows one or more transgenes to be provided in a single reprogramming vector, and various inducible or constitutive promoters can be combined together as an expression cassette to impart a more granular level of transcriptional control for the plurality of transgenes. These more specific levels of control can benefit the reprogramming process considerably, and separate expression cassettes on a vector can be designed accordingly as under the control of separate promoters.

Although there are advantages to providing such factors via a single, or small number of vectors, upper limitations on vector size do exist, which can stymie attempts to promote their delivery in a host target cell. For example, early reports on the use of polycistronic vectors were notable for extremely poor efficiency of reprogramming, sometimes occurring in less than 1% of cells, more typically less than 0.1%. These obstacles are due, in-part, to certain target host cells possessing poor tolerance for large constructs (e.g., fibroblasts), or inefficient processing of IRES sites by the host cells. Moreover, positioning of a factor in a vector expression cassette affects both its stoichiometric and temporal expression, providing an additional variable impacting reprogramming efficiency. Thus, some improved techniques can rely on multiple vectors each encoding one or more reprogramming factors in various expression cassettes. Under these designs, alteration of the amount of a particular vector for delivery provides a coarse, but relatively straightforward route for adjusting expression levels in a target cell.

In an alternate embodiment, the methods described herein do not require the somatic or non-embryonic cell to be contacted by a reprogramming factor.

Differentiation of an Induced Pluripotent Stem Cell

One aspect of the invention described herein provides a method to generate differentiated cells comprising delivering to a pluripotent cell population an agent that upmodulates SIRT2 and culturing the population under differentiating conditions for a period of time sufficient to generate at least one differentiated cell. In one embodiment, the method further comprises delivering an agent that downmodulates SIRT1

Pluripotent stem cells comprise the capacity to differentiate into any cell type of the organism. It should be understood that the methods and protocols for differentiating a stem cell will vary based on the cell type, e.g., differentiation into a neuron may require a different protocol compared to differentiation into a hepatocyte. Protocols for differentiating a stem cell into a given cell type are known in the art. The skilled practitioner is able to determine if a cell has differentiated into a particular cell type (e.g., a neuron) by assessing the differentiated cells for specific linage-derived markers (e.g., Class III (3-tubulin, neuron specific enolase (NSE), or calretinin). Markers for various cell types are known and can be determine by the skilled practitioner.

Specific differentiation conditions typically require cultureing in specific differentiation medium. As used herein, “differentiation media” refers to a medium containing factors required for differentiating a stem cell into a particular cell type. Differentiated media useful for generating a particular differentiated cell (e.g., a neuron, or other neuronal cell type) are commercially available for various cell types, e.g, at Cell Applications, Inc., San Diego, Calif. The skilled artisan can determine the appropriate differentiation media and conditions for a desired cell type.

In one embodiment, differentiating conditions are specific for neuronal differentiation (e.g., differentiation in to a neuronal progenitor cell). Methods for differentiation of a stem-like cell to a neuronal cell include culturing an adherent population of stem-like cell in a medium containing factors that promote neural differentiation, such as retinoic acid, BMP inhibitors (e.g., noggin), N2, B27, and ITS. The adherent stem-like cells can be adherent to a matrix, e.g, laminin, fibronection, or collagen, or adherent to a population of feeder cells, e.g., a monolayer of fibroblast cells. When cells in culture begin to commit to neural fates, e.g., as observed by the presence of neural rosettes, they are cultures in a permissive medium, and neuronal rosettes are passaged in permissive medium containing high levels of basic FGF2. Methods for neuronal differentiation are further are reviewed in, e.g., Dhara, S K., and Stice, S L. J Cell Biochem. 2008 Oct. 15; 105(3): 633-640, which is incorporated herein by reference in its entirety.

By way of another example, stem-like cells can be differentiated into a hepatocyte by culturing the stem-like cells in medium containing factors that promote hepatocyte differentiation, e.g., FGF-4, and HGF. After 6 days, the cells are cultured in medium containing FGF-4, HGF, and oncostatin M to allow for differentiation. Complete hepatocyte differentiation can be determined by assessing the cells for hepatocyte markers, such as GATA4, HNF4a, and albumin. Methods for hepatocyte differentiation are further are reviewed in e.g., Agarwak, S., et al. Stem Cells. 2008 Feb. 21; 26(5): 1117-1127, which is incorporated herein by reference in its entirety.

The stem cells for use with the methods and compositions described herein can be naturally occurring stem cells or “induced” stem cells, such as induced pluripotent stem cells generated using methods described herein. Induced pluripotent stem cells can be generated using any methods known in the art (e.g., as described herein). Stem cells can be obtained or generated from any mammalian subjects, e.g. human, primate, equine, bovine, porcine, canine, feline, rodent, e.g. mice, rats, hamster, etc. In one embodiment, the stem cell is a human stem cell. In one embodiment, the stem cell is a non-human stem cell.

In one embodiment, the pluripotent stem cell population is an embryonic stem cell population, an adult stem cell population, an induced pluripotent stem cell population, or a cancer stem cell population. In one embodiment, the stem cell is a non-embryonic stem cell.

In one embodiment, a pluripotent cell population is cultured in, e.g., differentiation media, for a period of time sufficient to generate at least one differentiated cell. Culturing can occur for a period of from 1-5 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 30 days, at least 40 days, at least 50 days, at least 60 days, at least 70 days, at least 80 days, at least 90 days, at least 100 days, at least 110 days, at least 120 days, at least 130 days, at least 140 days, at least 150 days, at least 160 days, at least 170 days, at least 180 days, at least 190 days, at least 200 days, at least 210 days, at least 220 days, at least 230 days, at least 240 days, at least 250 days, at least 260 days, at least 1270 days, at least 280 days, at least 290 days, at least 300 days, or more. In one embodiment, culturing occurs for a period of 7 to 100 days, 7 to 200 day, 7 to 300 days, 100 to 200 days, 200 to 300 days, 50 to 150 days, 150 to 250 days, or 150 to 300 days.

In one embodiment, the methods described herein produce an enhanced number of differentiated cells by at least 2-fold as compared to an appropriate control. In another embodiment, the methods described herein result in an enhancement of the number of differentiated cells by at least 3-fold, at least 4-fold, at least 5-fold, at least 6-fold, at least 7-fold, at least 8-fold, at least 9-fold, at least 10-fold, or more as compared to an appropriate control. In one embodiment, enhancement is by at least 100×, 250×, 500×, 750×, 100× or more, as compared to an appropriate control. One such “appropriate control” is a similar or identical cell subjected to an otherwise identical method that does not downmodulate SIRT1 and/or upmodulate SIRT2. The efficiency of de-differentiation can be assessed as described above for the efficiency of reprogramming.

In one embodiment, the differentiated cells are produced in a significantly shorter period of time than in appropriate control. In one embodiment, the period of time is at least 10% shorter as compared to an appropriate control. In one embodiment, period of time is at least 20%, at least 30%, at least 40%, at least 50%, at least 60%, at least 70%, at least 80%, at least 90%, at least 99%, or more, shorter as compared to an appropriate control.

Another aspect of the invention relates to a cell line comprising differentiated cells generated by any of the methods described herein.

Agents

In various embodiment, agents are delivered to cells to modulate (e.g., upmodulate, or downmodulate) SIRT1 and SIRT2. The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

Such an agent can take the form of any entity which is normally not present or not present at the levels being administered in the cell. Agents such as chemicals; small molecules; nucleic acid sequences; nucleic acid analogues; proteins; peptides; aptamers; antibodies; or fragments thereof, can be identified or generated for use to downmodulate or upmodulate SIRT1 or SIRT2.

Agents in the form of nucleic acid sequences designed to specifically inhibit gene expression are particularly useful. Such a nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

In one embodiment the agent is a catalytic antisense nucleic acid constructs, such as ribozymes, which is capable of cleaving RNA transcripts and thereby preventing the production of the encoded protein. Ribozymes are targeted to and anneal with a particular sequence by virtue of two regions of sequence complementary to the target flanking the ribozyme catalytic site. After binding the ribozyme cleaves the target in a site specific manner. The design and testing of ribozymes which specifically recognize and cleave sequences of the specific gene products is commonly known to persons of ordinary skill in the art.

In one embodiment, the agent inhibits gene expression (i.e. suppress and/or repress the expression of the gene). Such agents are referred to in the art as “gene silencers” and are commonly known in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNA, siRNA, micro RNAi (miRNA), antisense oligonucleotides, etc.

The agent may function directly in the form in which it is administered. Alternatively, the agent can be modified or utilized intracellularly to produce something which modulates SIRT1 or SIRT2, such as introduction of a nucleic acid sequence into the cell and its transcription resulting in the production of the nucleic acid and/or protein inhibitor or activator of SIRT1 or SIRT2 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

Agents in the form of a protein and/or peptide or fragment thereof can also be designed to downmodulate or upmodulate SIRT1 or SIRT2. Such agents encompass proteins which are normally absent or proteins that are normally endogenously expressed in the host cell. Examples of useful proteins are mutated proteins, genetically engineered proteins, peptides, synthetic peptides, recombinant proteins, chimeric proteins (any of which may take the form of a dominant negative protein for SIRT1 or SIRT2), antibodies, midibodies, minibodies, triabodies, humanized proteins, humanized antibodies, chimeric antibodies, modified proteins and fragments thereof. Agents also include antibodies (polyclonal or monoclonal), neutralizing antibodies, antibody fragments, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified.

In one embodiment, an agent that downmodulates SIRT2 is delivered to a differentiated cell to a generate at least one induced pluripotent stem cell. In such embodiment, the agent downmodulates SIRT2 by at least 10%, by at least 20%, by at least 30%, by at least 40%, by at least 50%, by at least 60%, by at least 70%, by at least 80%, by at least 90%, by at least 100% or more as compared to an appropriate control. In an alternate embodiment, an agent that upmodulates SIRT2 is delivered to a stem cell to generate at least one differentiated cell. In such embodiment, the agent upmodulates SIRT2 by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% as compared to an appropriate control. An “appropriate control” can be the same type of cell or population thereof similarly or identically treated to which an agent has not been delivered.

In another embodiment, an agent that downmodulates SIRT1 is delivered to a stem cell to generate at least one differentiated cell. In such embodiment, the agent downmodulates SIRT1 by at least 10%, by at least 20%, by at least 30%, by at least 40%, by at least 50%, by at least 60%, by at least 70%, by at least 80%, by at least 90%, by at least 100% as compared to an appropriate control. In an alternate embodiment, an agent that upmodulates SIRT1 is delivered to a differentiated cell to de-differentiate the cell (e.g., generate at least one induced pluripotent stem cell). In such embodiment, the agent upmodulates SIRT1 by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% or more as compared to an appropriate control. An “appropriate control” can be a cell or population thereof similarly or identically treated to which an agent has not been delivered.

In one embodiment, SIRT1 is upmodulated by a nucleic acid encoding SIRT1 expressed in the cell e.g., via a vector comprising a nucleic acid encoding SIRT1. In another embodiment, a nucleic acid encoding SIRT1 is expressed in the cell e.g., via expression of a nucleic acid encoding SIRT1 as naked DNA. In one embodiment, the nucleic acid encoding SIRT1 has a sequence corresponding to the sequence of SEQ ID NO: 2; or comprises the sequence of SEQ ID NO: 2; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 2, and having the same activity as the sequence of SEQ ID NO: 2 (e.g., acetylation of its substrates).

In one embodiment, SIRT2 is upmodulated by expression of a nucleic acid encoding SIRT1. The nucleic acid encoding SIRT2 can be expressed in the cell e.g., via a vector comprising a nucleic acid encoding SIRT2. In another embodiment, a nucleic acid encoding SIRT2 is expressed in the cell e.g., via expression of a nucleic acid encoding SIRT2 as naked DNA. In one embodiment, the nucleic acid encoding SIRT2 has a sequence corresponding to the sequence of SEQ ID NO: 3; or comprises the sequence of SEQ ID NO: 3; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 3, and having the same activity as the sequence of SEQ ID NO: 3 (e.g., acetylation of its substrates).

In one embodiment, the agent is a small molecule that downmodulates SIRT1 or SIRT2. Such small molecules include, but are not limited, to the small molecules listed in Table 1. Methods for screening small molecules are known in the art and can be used to identify a small molecule that is efficient at, for example, inducing pluripotent stem cells or differentiated cells, given the desired target (e.g., SIRT1 or SIRT2).

TABLE 1
Small molecule compounds targeting Sirtuins

| | Molecular | | |
|---|---|---|---|
| Full Name | Weight | Formula | Information |
| SRT1720 | 506.02 | C25H23N7OS•HCl | SRT1720 HCl is a selective SIRT1 activator with EC50 of |
| | | | 0.16 μM in a cell-free assay, but is >230-fold less potent |
| | | | for SIRT2 and SIRT3 |
| EX527 | 248.71 | C13H13ClN2O | EX 527 is a potent and selective SIRT1 inhibitor with IC50 |
| | | | of 38 nM in a cell-free assay, exhibits >200-fold selectivity |
| | | | against SIRT2 and SIRT3. Phase 2. |
| Sirtinol | 394.47 | C26H22N2O2 | Sirtinol is a specific SIRT1 and SIRT2 inhibitor with IC50 |
| | | | of 131 μM and 38 μM in cell-free assays, respectively. |
| Nicotinamide | 122.12 | C6H6N2O | Nicotinamide (Vitamin B3), a water-soluble vitamin, is an |
| (Vitamin B3) | | | active component of coenzymes NAD and NADP, and also |
| | | | act as an inhibitor of sirtuins. |
| SRT2183 | 468.57 | C27H24N4O2S | SRT2183 is a small-molecule activator of the sirtuin |
| | | | subtype SIRT1, currently being developed by Sirtris |
| | | | Pharmaceuticals. |
| Tenovin-6 | 454.63 | C25H34N4O2S | Tenovin-6 acts through inhibition of the protein- |
| | | | deacetylating activities of SirT1 and SirT2. Tenovin-6 |
| | | | inhibits the protein deacetylase activities of purified human |
| | | | SIRT1, SIRT2, and SIRT3 in vitro with IC50 of 21 μM, 10 μM, |
| | | | and 67 μM, respectively. |
| SRT2104 | 516.64 | C26H24N6O2S2 | SRT2104 (GSK2245840) is a selective SIRT1 activator |
| (GSK2245840) | | | involved in the regulation of energy homeostasis. Phase 2. |
| Thiomyristoyl | 581.85 | C34H51N3O3S | Thiomyristoyl is a potent and specific SIRT2 inhibitor with |
| | | | an IC50 of 28 nM. It inhibits SIRT1 with an IC50 value of |
| | | | 98 μM and does not inhibit SIRT3 even at 200 μM. |
| SirReal2 | 420.55 | C22H20N4OS2 | SirReal2 is a potent and selective Sirt2 inhibitor with IC50 |
| | | | of 140 nM. |
| Salermide | 394.47 | C26H22N2O2 | Salermide is a reverse amide with a strong in vitro |
| | | | inhibitory effect on Sirt1 and Sirt2. Compared with Sirt1, |
| | | | Salermide is even more efficient at inhibiting Sirt2. |
| AGK2 | 434.27 | C23H13Cl2N3O2 | AGK2 is a potent, and selective SIRT2 inhibitor with IC50 |
| | | | of 3.5 μM that minimally affects either SIRT1 or SIRT3 at |
| | | | 10-fold higher levels. |
| SRT3025 | 606.2 | C31H31N5O2S2•HCl | SRT3025 is an orally available small molecule activator of |
| | | | the SIRT1 enzyme. |
| Fisetin | 286.24 | C15H10O6 | Fisetin (Fustel) is a potent sirtuin activating compound |
| | | | (STAC) and an agent that modulates sirtuins. |
| Quercetin | 302.24 | C15H10O7 | Quercetin, a natural flavonoid present in vegetables, fruit |
| | | | and wine, is a stimulator of recombinant SIRT1 and also a |
| | | | PI3K inhibitor with IC50 of 2.4-5.4 μM. Phase 4. |

In one embodiment, the agent that downmodulates SIRT1 or SIRT2 is an antibody or antigen-binding fragment thereof, or an antibody reagent. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

In some embodiments, a nucleic acid for use as an agent as described herein (e.g. SIRT1, or SIRT2) is contained in a vector for delivery and/or expression of the nucleic acid.

In one embodiment, the agent that downmodulates SIRT1 or SIRT2 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., SIRT1 or SIRT2. Thus, oligonucleotides are chosen that are sufficiently complementary to the target, i.e., that hybridize sufficiently well and with sufficient specificity in the context of the cellular environment, to give the desired effect.

In one embodiment the agent downmodulates SIRT1 or SIRT2 by RNA inhibition. Inhibitors of the expression of a given gene can be an inhibitory nucleic acid. In oneembodiment, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. SIRT1 or SIRT2. In one embodiment, the agent is siRNA that downmodulates SIRT1 or SIRT2. In one embodiment, the agent is shRNA that downmodulates SIRT1 or SIRT2.

The skilled practitioner is able to design siRNA, shRNA, or miRNA to target SIRT1 or SIRT2, e.g., using publically available design tools. siRNA, shRNA, or miRNA is commonly commercially made by companies such as Dharmacon (Layfayette, Colo.) or Sigma Aldrich (St. Louis, Mo.). One skilled in the art will be able to readily assess whether the siRNA, shRNA, or miRNA effective target e.g., SIRT1 or SIRT2, for its downregulation, for example by transfecting the siRNA, shRNA, or miRNA into cells and detecting the expression levels of a gene within the cell via western-blotting for the encoded protein.

In one embodiment, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

microRNA

In one embodiment, the agent that downmodulates SIRT1 or SIRT2 is miRNA. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

In one embodiment, the agent that downmodulates SIRT2 is miRNA-200c-5p. miRNA-200c-5p is the mature product of miRNA-200c. miRNA-200c-5p sequences are known for a number of species, e.g., human miRNA-200c-5p, e.g., miRBase Accession number MIMAT0004657. Human miRNA-200c-5p comprises the sequence of CGUCUUACCCAGCAGUGUUUGG (SEQ ID NO: 1). miRNA-200c-5p can refer to human miRNA-200c-5p, including naturally occurring variants, molecules, and alleles thereof.

In one embodiment, the agent, e.g., the miRNA, has a sequence corresponding to the sequence of SEQ ID NO: 1; or comprises the sequence of SEQ ID NO: 1; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 1, and having the same activity as the sequence of SEQ ID NO: 1 (e.g., downmodulates SIRT2, and induces a pluripotent state).

Various other microRNAs (e.g., as miR-302, and -367) have been shown synergize with the reprogramming factors. One or more of these can also be delivered to the cells to induce de-differentiation in the methods described herein. The miR-302/367 cluster contains eight microRNAs, miR-367, 302d, 302c-5p, 302c-3p, 302a-5p, 302a-3p, 302b-5p and 302b-3p. miR302a-d contain the same seed sequence, AAGUGCU (SEQ ID NO: 200). The miR-302/367 cluster members have been demonstrated to play an important role in diverse biological processes, such as the pluripotency of human embryonic stem cells (hESCs), self-renewal and reprogramming. The miR-200 cluster is a family of microRNAs that includes miR-200a, miR-200b, miR-200c, miR-141 and miR-429. In one embodiment, the methods described herein do not include/deliver the members of the miRNA-200 cluster other than miRNA-200c-5p.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 90%, at least 91%, at least 92%, at least 93%, at least 94%, at least 95%, at least 96%, at least 97%, at least 98%, at least 99%, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

Delivery of an Agent

In the herein described methods and compositions, the agent is contacted to the cell such that it can exert its intended effect on the cell. In one embodiment, the agent exerts its effects on cells merely by interacting with the exterior of the cell (e.g., by binding to a receptor). Agents that act on the cell internally (e.g., RNAi or encoded protein) may be delivered in a form readily taken up by the cell when contacted to the cell (e.g., in a formulation which facilitates cellular uptake and delivery to the appropriate subcellular location). In one embodiment, the agent is in a formulation in which it is readily taken up by the cell so that it can exert it effect. In one embodiment, the agent is applied to the media, where it contacts the cell (such as the progenitor and/or feeder cells) and produces its modulatory effects.

The agent may result in gene silencing of the target gene (e.g., SIRT1 or SIRT2), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%.

As used herein, “delivery” refers to an effective amount of, e.g., an agent, that enters a cell or population thereof, and properly functions, e.g., delivery of functional protein or a vector that appropriately expresses the agent. Delivery can be done using any technique known in the art. Exemplary techniques include, but are not limited to transduction, nucleofection, electroporation, direct injection, or transfection. Effective delivery of an agent (e.g., a vector encoding SIRT1 or SIRT2, or a small molecule inhibitor of SIRT1 or SIRT2) can be assessed by measuring protein or mRNA levels, e.g., via Westerblotting or qRT-PCR, respectively. Effective delivery of an agent can additionally be measured by assessing the biological function of the intended target of the agent.

In one embodiment, an agent is delivered to a cell via culturing the cell in a medium comprising the agent. Culturing a population of cells with one or more agents can be achieved in a variety of ways. For instance, a population of cells, e.g., somatic or non-embryonic cells, may be contacted with one or more agents. Somatic or non-embryonic cells can be cultured in the presence of these agents for a period of time, such as for seven or more days. When more than one agent (e.g., an agent that downmodulates SIRT2, and an agent that upmodulates SIRT1) is in contact with a population of cells, the agents can be present in the cell culture medium together, such that the cells are exposed to the agents simultaneously. Alternatively, the agents may be added to the cell culture medium sequentially. For instance, the one or more agents may be added to a population of cells in culture according to a particular regimen, e.g., such that different agents are added to the culture media at different times during a culture period.

It is understood that the optimal method for delivery can vary based on the type of agent, and can be determined by a skilled practitioner.

Identifying Cell Populations of a Particular Cell Fate

One aspect of the invention relates to a method for selecting pluripotent stem cells from an induced population comprising measuring the level and/or activity of SIRT1 and SIRT2 in a population of candidate cells, and selecting cells that exhibit an increased level and/or activity of SIRT1 and decreased level and/or activity of SIRT2. In one embodiment, the candidate cells were induced using any of the methods described herein. In another embodiment, the candidate cells were induced using any method known in the art.

In one embodiment, the level and/or activity of SIRT1 is increased by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% or more as compared to an appropriate control, and the level and/or activity of SIRT2 is decreased by at least 10%, by at least 20%, by at least 30%, by at least 40%, by at least 50%, by at least 60%, by at least 70%, by at least 80%, by at least 90%, by at least 100% as compared to an appropriate control. As used herein, an “appropriate control” refers to a similarly or identically treated cell or population thereof that is not an induced pluripotent cell. An appropriate control can be an identical cell population that was not induced to a pluripotent state, e.g., a cell population that was not contacted by an agent or reprogramming factor.

Another aspect of the invention described herein provides a method for selecting differentiated cells from an induced population comprising measuring the level and/or activity of SIRT1 and SIRT2 in a population of candidate cells, and selecting cells that exhibit an increased level and/or activity of SIRT2 and decreased level and/or activity of SIRT1. In one embodiment, the candidate cells are induced using any of the methods described herein. In another embodiment, the candidate cells are induced using any method known in the art.

In one embodiment, the level and/or activity of SIRT2 is increased by at least 2-fold, by at least 3-fold, by at least 4-fold, by at least 5-fold, by at least 6-fold, by at least 7-fold, by at least 8-fold, by at least 9-fold, by at least 10-fold or more as compared to an appropriate control, or by at least 10%, by at least 20%, by at least 30%, by at least 40%, by at least 50%, by at least 60%, by at least 70%, by at least 80%, by at least 90%, by at least 100% or more as compared to an appropriate control, and the level and/or activity of SIRT1 is decreased by at least 10%, by at least 20%, by at least 30%, by at least 40%, by at least 50%, by at least 60%, by at least 70%, by at least 80%, by at least 90%, by at least 100% as compared to an appropriate control. As used herein, an “appropriate control” can be a stem cell or population thereof, either naturally occurring or induced. An appropriate control can be an identical stem cell population that was not induced to be differentiated, e.g., a cell population that was not contacted by an agent or differentiation factor, but otherwise identically treated.

In one embodiment, the levels of SIRT1 and/or SIRT2 is measured via immunofluorescence using a reagent (e.g., an antibody reagent) that detects SIRT1 or SIRT2 protein in the cell. Fluorescence-activated cell sorting (FACS) can be used to select for cells with a given SIRT1 and SIRT2 expression level. Alternatively, levels of SIRT1 and/or SIRT2 can be measured, e.g., by assessing the protein level or mRNA level in the cell via, e.g., Westernblotting or PCR-based screening (e.g., qRT-PCR), respectively. Activity of SIRT1 and/orSIRT2 can be assessed e.g., via functional assays, e.g., by determining if SIRTlor SIRT2 substrates are acetylated.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. A method to generate induced human pluripotent stem cells comprising delivering to a somatic or non-embryonic cell population an effective amount of one or more reprogramming factors and also an agent that downmodulates SIRT2, and culturing the somatic or non-embryonic cell population for a period of time sufficient to generate at least one induced human pluripotent stem cell.
2. The method of paragraph 1, further comprising delivering to the somatic or non-embryonic cell population an effective amount of an agent that upmodulates SIRT1.
3. The method of paragraph 1 or 2, wherein the reprogramming factor is an agent that increases the expression of c-Myc, Oct4, Sox2, Nanog, Lin-28, or Klf4 in the cells.
4. The method of paragraph 1-3, wherein the reprogramming factor is an agent that increases the expression of SV40 Large T Antigen (“SV40LT”), or short hairpin RNAs targeting p53 (“shRNA-p53”).
5. The method of any of paragraphs 1-3, wherein the agent that downmodulate SIRT2 is selected from the group consisting of a small molecule, an antibody, a peptide, an antisense oligonucleotide, and an RNAi.
6. The method of paragraph 5, wherein the RNAi is a microRNA, an siRNA, or a shRNA.
7. The method of paragraph 6, wherein the microRNA is miR-200c-5p.
8. The method of any one of paragraphs 2-7, wherein the agent that upmodulates SIRT1 is selected from the group consisting of a small molecule, a peptide, and an expression vector encoding SIRT1.
9. The method of any one of paragraphs 1-8, further comprising delivering to the cells one or more microRNAs selected from the miR-302/367 cluster.
10. The method of any one of paragraphs 1-9, wherein delivery comprises contacting the cell population with an agent or a vector that encodes the agent.
11. The method of any one of paragraphs 1-10, wherein delivery comprises transduction, nucleofection, electroporation, direct injection, and/or transfection.
12. The method of paragraph 10, wherein the vector is non-integrative or integrative.
13. The method of paragraph 12, wherein the non-integrative vector is selected from the group consisting of an episomal vector, an EBNA1 vector, a minicircle vector, a non-integrative adenovirus, a non-integrative RNA, and a Sendai virus.
14. The method of paragraph 10-12, wherein the vector is an episomal vector.
15. The method of paragraph 10, wherein the vector is a lentivirus vector.
16. The method of any one of paragraphs 1-15, wherein the culturing is for a period of from 7 to 21 days.
17. The method of any one of paragraphs 1-16, wherein SIRT2 is downmodulated by at least about 50%, 60%, 70%, 80% or 90% as compared to an appropriate control.
18. The method of any one of paragraphs 1-17, wherein SIRT1 is upmodulated by at least about 2×, 5×, 6×, 7×, 8×, 9×, or 10× as compared to an appropriate control.
19. The method of any one of paragraphs 1-18, wherein at least a 2× enhancement of the number of induced pluripotent stem cells is produced as compared to an appropriate control.
20. A cell line comprising induced pluripotent stem cells generated by the method of any one of paragraphs 1-19.
21. A pharmaceutical composition comprising an induced pluripotent stem cell or population thereof generated by the method of any one of paragraphs 1-19, and a pharmaceutically acceptable carrier.
22. A method to generate differentiated cells comprising delivering to a pluripotent cell population an agent that upmodulates SIRT2 and culturing the population under differentiating conditions for a period of time sufficient to generate at least one differentiated cell.
23. The method of paragraph 22, further comprising delivering to the pluripotent cell population an agent that downmodulates SIRT1.
24. The method of paragraph 22 or 23, wherein the pluripotent cell population is selected from the group consisting of an embryonic stem population, an adult stem cell population, an induced pluripotent stem cell population, and a cancer stem cell population.
25. The method of paragraph 23 or 24, wherein the agent that downmodulates SIRT1 is selected from the group consisting of a small molecule, an antibody, a peptide, an antisense oligonucleotide, and an RNAi.
26. The method of paragraph 25, wherein the RNAi is a microRNA, an siRNA, or a shRNA.
27. The method of any one of paragraphs 22-26, wherein the agent that upmodulates SIRT2 is selected from the group consisting of a small molecule, a peptide, and an expression vector encoding SIRT2.
28. The method of any one of paragraphs 22-27, wherein delivery comprises contacting the cell population with a vector that encodes the agent.
29. The method of paragraph 28, wherein delivery comprises transduction, nucleofection, electroporation, direct injection, and/or transfection.
30. The method of paragraph 28, wherein the vector is non-integrative or integrative.
31. The method of paragraph 30, wherein the non-integrative vector is selected from the group consisting of an episomal vector, an EBNA1 vector, a minicircle vector, a non-integrative adenovirus, a non-integrative RNA, and a Sendai virus.
32. The method of any of paragraphs 28-30, wherein the vector is an episomal vector.
33. The method of paragraph 28, wherein the vector is a lentivirus vector.
34. The method of any one of paragraphs 22-33, wherein the culturing is for a period of from 7 to 300 days.
35. The method of any one of paragraphs 22-33, wherein SIRT1 is downmodulated by at least about 50%, 60%, 70%, 80% or 90% as compared to an appropriate control.
36. The method of any one of paragraphs 23-35, wherein SIRT2 is upmodulated by at least about 2×, 5×, 6×, 7×, 8×, 9×, or 10× as compared to an appropriate control.
37. The method of any one of paragraphs 23-36, wherein at least a 2× enhancement of the number of differentiated cells is produced as compared to an appropriate control.
38. The method of any one of paragraphs 23-37, wherein the differentiated cells are produced in a significantly shorter period of time as compared to an appropriate control.
39. The method of any of paragraphs 22-38, wherein the differentiating conditions are specific for neuronal differentiation to thereby generate neuronal cells.
40. A cell line comprising differentiated cells generated by the method of any one of paragraphs 22-39.
41. A method for selecting pluripotent stem cells from an induced population comprising measuring the level and/or activity of SIRT1 and SIRT2 in a population of candidate cells, and selecting cells which exhibit an increased level and/or activity of SIRT1 and decreased level and/or activity of SIRT2.
42. The method of paragraph 41, wherein the level and/or activity of SIRT1 is increased by at least about 2×, 5×, 6×, 7×, 8×, 9×, or 10× as compared to an appropriate control.
43. The method of paragraph 41, wherein the level and/or activity of SIRT2 is decreased by at least about 50%, 60%, 70%, 80% or 90% as compared to an appropriate control.
44. The method of paragraph 41, wherein the candidate cells are induced by the method of any of paragraphs 1-21.
45. A method for selecting differentiated cells from an induced population comprising measuring the level and/or activity of SIRT1 and SIRT2 in a population of candidate cells, and selecting cells which exhibit an increased level and/or activity of SIRT2 and decreased level and/or activity of SIRT1.
46. The method of paragraph 45, wherein the level and/or activity of SIRT2 is increased by at least about 2×, 5×, 6×, 7×, 8×, 9×, or 10× as compared to an appropriate control.
47. The method of paragraph 45, wherein the level and/or activity of SIRT1 is decreased by at least about 50%, 60%, 70%, 80% or 90% as compared to an appropriate control.
48. The method of paragraph 45, wherein the candidate cells are differentiated by the method of any of paragraphs 50-53.
49. The method of paragraph 41 or 45, wherein measuring is by immunofluorescence.

### EXAMPLES

A hallmark of cancer cells is the metabolic switch from oxidative phosphorylation (OXPHOS) to glycolysis, a phenomenon referred to as the “Warburg effect”, which is also observed in primed human pluripotent stem cells (hPSCs) such as human embryonic stem cells (hESCs) and human induced pluripotent stem cells (hiPSCs). It is reported herein that downregulation of SIRT2 and upregulation of SIRT1 is a molecular signature of primed hPSCs and critically regulates induced pluripotency. SIRT2 downregulation leads to hyperacetylation of enzymes of the glycolytic pathway (e.g., aldolase, glyceraldehyde-3-phosphate dehydrogenase, phosphoglycerate kinase, and enolase) and to their enhanced activities, indicating that SIRT2 critically regulates metabolic reprogramming during induced pluripotency. In support of this model, knockdown of SIRT2 in human fibroblasts resulted in significantly decreased OXPHOS and increased glycolysis, both in the absence and presence of reprogramming factors. Aldolase lysine residue 322 was identified herein as an important acetylation site whose deacetylation by SIRT2 robustly downregulates aldolase activity. In addition, it was found that miR-200c-5p specifically targets SIRT2, downregulating its expression through two miRNA-response elements that are identified to reside within the coding sequence. Furthermore, doxycycline-induced SIRT2 overexpression in hESCs significantly affected energy metabolism, altering stem cell function such as pluripotent differentiation properties. Taken together, experimental data described herein identify the miR-200c-SIRT2 axis as a key regulator of metabolic reprogramming (Warburg-like effect), at a minimum, in part via regulation of glycolytic enzymes acetylation and activities, during human induced pluripotency, as well as pluripotent stem cell function.

### INTRODUCTION

Recent proteomics studies revealed that numerous proteins of the nucleus, cytoplasm, and mitochondria involved in diverse aspects of cellular metabolism are highly acetylated in human, mouse, and prokaryotic cells¹⁴⁻¹⁶. In particular, virtually all enzymes involved in glycolysis and the tricarboxylic acid (TCA) cycle were found to be acetylated in human liver tissues¹⁵, strongly suggesting that protein acetylation is a key mechanism regulating metabolism¹⁷, which prompted the hypothesis that protein acetylation regulates, at least in part, metabolic reprogramming. Protein acetylation can be modulated by histone acetyl transferase (HATs), as well as by class I, II, and III histone deacetylases (HDAC). Among these, class III HDACs, termed sirtuins, are NAD-dependent protein deacetylases that are highly conserved from bacteria to human^{18, 19}. Since sirtuins are the only HDACs whose activity is dependent on NAD, a critical co-factor of cell metabolism, it was further hypothesized that certain sirtuin members play important roles in regulating metabolic reprogramming and are likely linked to induced pluripotency and stem cell fate control. Experimental data provided herein indicate that altered acetylation levels of glycolytic enzymes by SIRT2 downregulation critically regulate metabolic reprogramming during human induced pluripotency and influence stem cell function and regulation in primed hPSCs.

Results

Warburg-Like Effect in hESCs and hiPSCs.

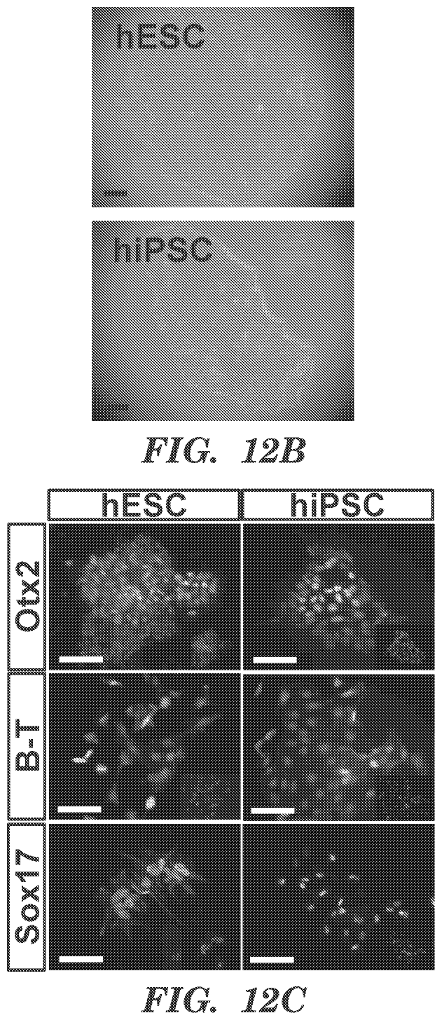

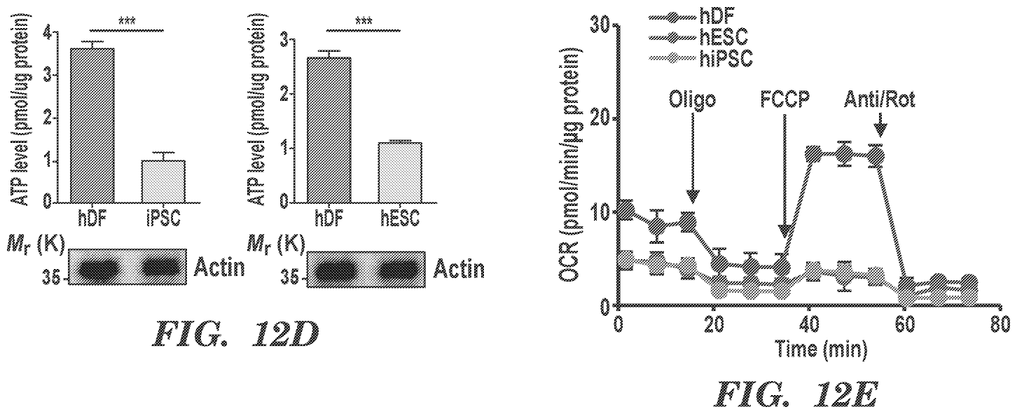

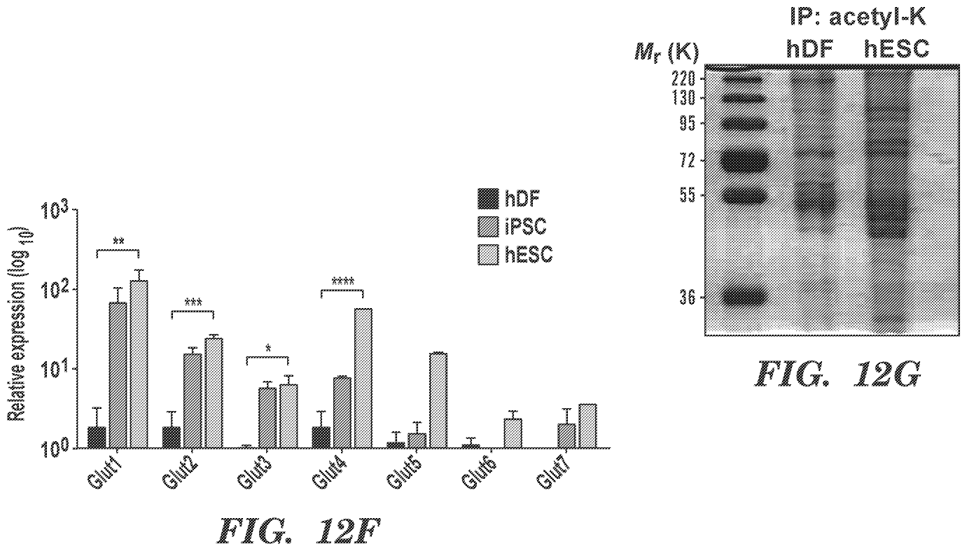

To compare energy metabolism between human pluripotent stem cells (hPSCs) and their somatic counterpart, human iPSCs from were derived from newborn dermal fibroblasts (hDFs) by introducing four reprogramming genes (c-Myc, Oct4, Sox2, and Klf4) using inducible lenti-viruses and confirmed robust expression of the canonical pluripotency markers (Oct4, Nanog, TRA1-60, and SSEA4) in the resulting hiPSCs and in hESCs (FIG. 12A). In addition, these hiPSCs and hESCs exhibited almost identical morphology such as large nuclei and scant cytoplasm, and showed pluripotent differentiation into all 3 germ layers (FIGS. 12B and 12C). Intracellular ATP levels were significantly lower in hESCs and hiPSCs compared to fibroblasts (FIG. 12D). Metabolic parameters were assayed using the Seahorse Flux analyzer by comparing mitochondrial respiration level defined as oxygen consumption rate (OCR)²⁰. When cells were treated with oligomycin, an inhibitor of ATP synthase, OCR was reduced more efficiently in fibroblasts than in hESCs and hiPSCs (FIG. 12E). Adding triflurocarbonylcyanide phenylhydrazone (FCCP), an uncoupling reagent maximizing oxygen consumption, resulted in significantly higher OCR in fibroblasts than in hESCs and hiPSCs, indicating a higher maximal respiratory capacity in fibroblasts (FIG. 12E), which was almost completely blocked by the addition of rotenone, an inhibitor of complex I. Since the Warburg effect is closely related to increased glucose uptake by upregulation of glucose transporters (GLUTs) in cancer cells²¹, the expression levels of GLUT genes were compared. As shown in FIG. 12F, the levels of GLUT1-4 mRNAs were significantly upregulated in both iPSCs and hESCs compared to fibroblasts. Taken together, these results, in line with previous findings^{11, 13, 22, 23,} demonstrate that a Warburg-like effect is operating in primed hPSCs.

Glycolytic Enzymes are Highly Acetylated in hPSCs.

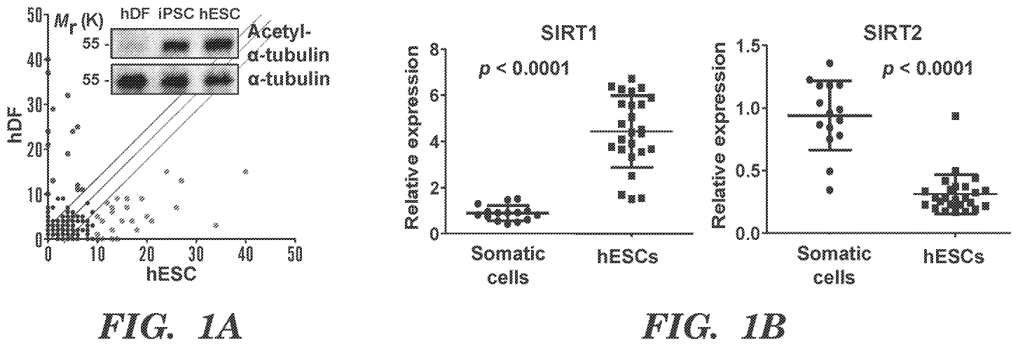

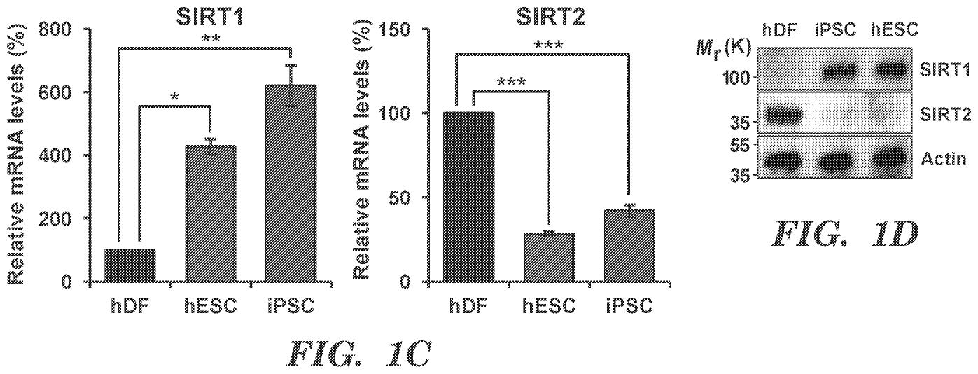

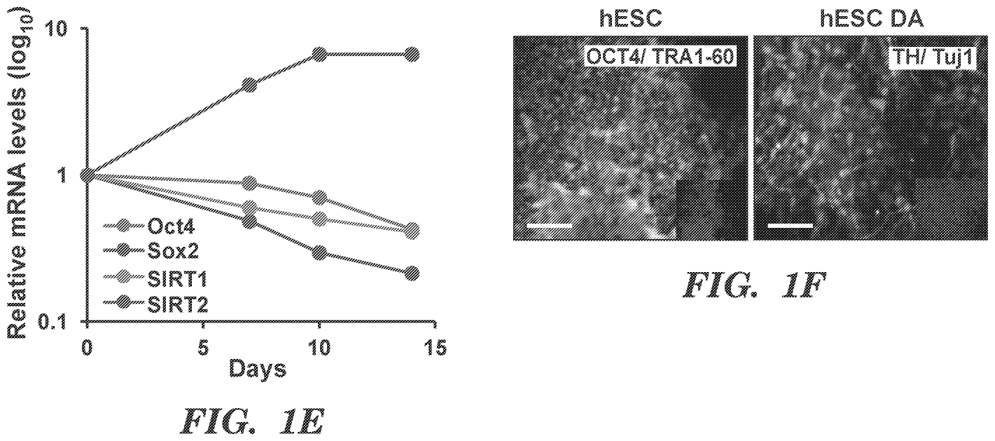

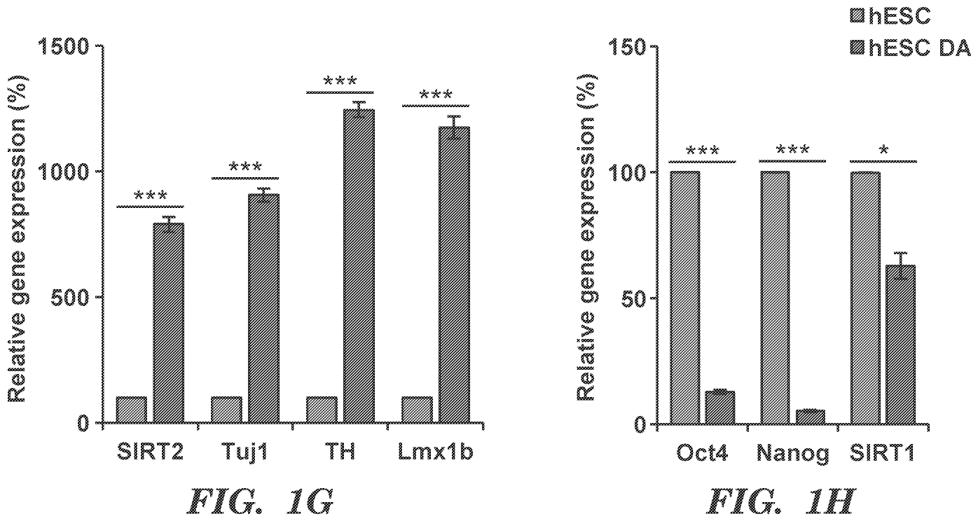

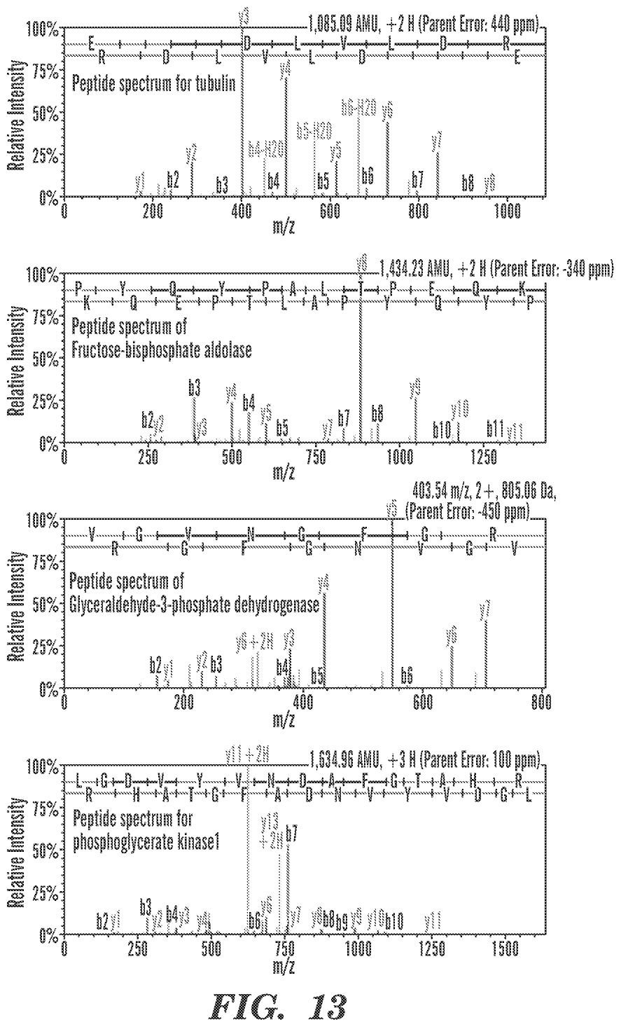

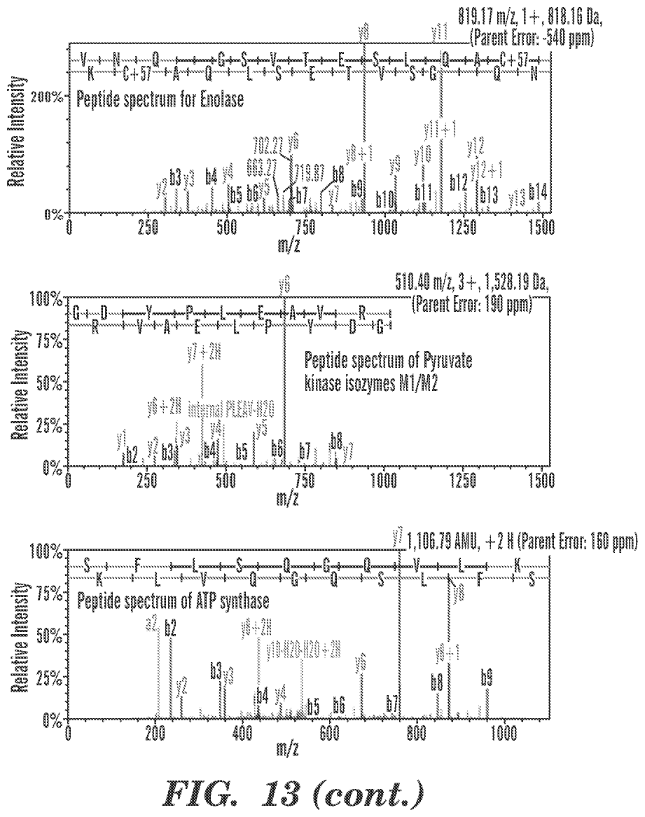

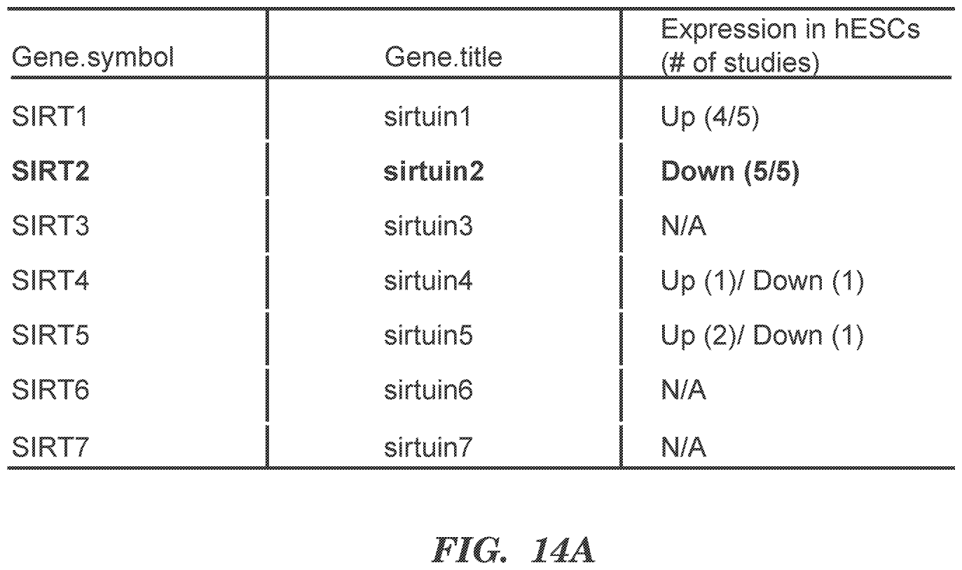

To address the hypothesis that regulation of acetylation affects the metabolic switch, protein acetylation in hESCs and dermal fibroblasts were compared. Acetylated proteins were pulled down by immunoprecipitation with acetyl-Lys antibody and subjected them to liquid chromatography-tandem mass spectrometry (LC-MS/MS) analyses following SDS-PAGE and in-gel trypsin digestion (FIG. 12G). This proteomic analysis identified >200 acetylated proteins in both hDFs and hESCs. To minimize non-specificity, proteins with less than 10 peptide hits were excluded (FIG. 1A), which represent highly stringent ID criteria (peptide or protein probability >95%, Exclusive spectrum count option in Scaffold4; found on the world wide web at http://www.proteomesoftware.com/). The graph in FIG. 1A illustrates this proteomic analysis where proteins with higher acetylation (>1.5 fold) in hESCs or in hDFs are shown. A total of 28 proteins were found to be highly acetylated (Table 2), and a total of 15 proteins are highly deacetylated (Table 3), in hESCs compared to fibroblasts. Two well-characterized SIRT2 substrates, tubulin α/β and 14-3-3 are among the highly acetylated proteins in hESCs^{24, 25}. In agreement with these results, western blot analyses confirmed that hESCs and hiPSCs contain higher levels of acetylated a-tubulin than hDFs while they express similar levels of total a-tubulin (FIG. 1A, inlet). Notably, this analysis revealed that 5 out of 10 glycolytic enzymes are highly acetylated in hESCs: aldolase (encoded by ALDOA), glyceraldehyde-3-phosphate dehydrogenase (encoded by GAPDH), phosphoglycerate kinase (encoded by PGK1), enolase (encoded by ENO1), and pyruvate kinases (encoded by PKM1 and (Table 2). Collision-induced dissociation (CID) spectra of the acetylated peptides derived from these glycolytic proteins are shown in FIG. 13.

Downregulation of SIRT2 and Upregulation of SIRT1 is a Molecular Signature of Primed hPSCs.

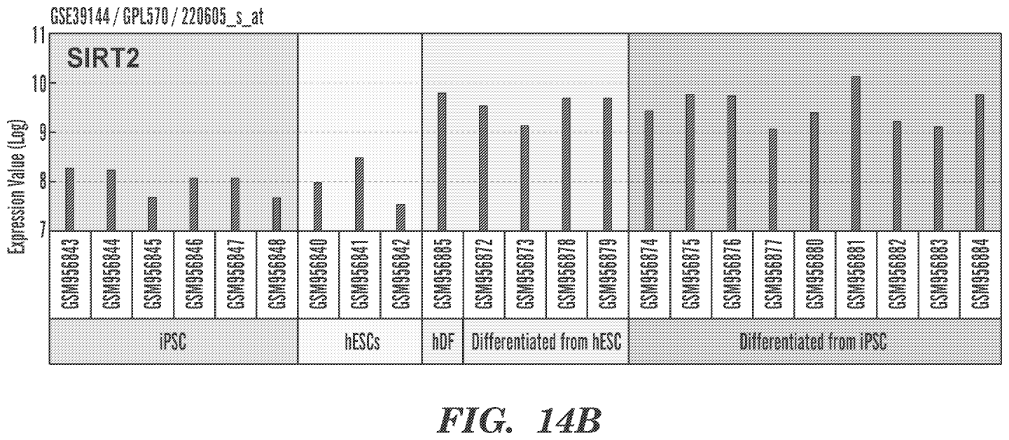

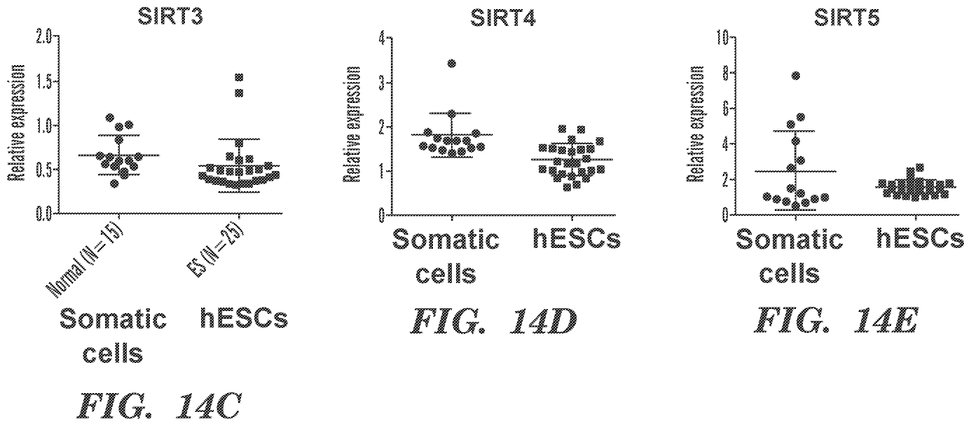

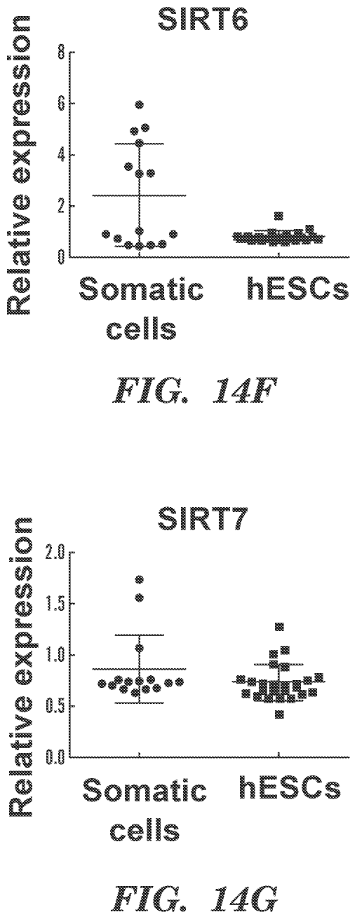

It was next determined if any acetylation-modulating factor(s) such as HATs or HDACs show a unique expression pattern in hPSCs compared to their counterpart somatic tissues by meta-analyses of web-based microarray databases. five independent studies (GSE28633²⁶, GSE18265²⁷, GSE20013²⁸, GSE39144 (found on the world wide web at http://www.ncbi.nlm.nih.gov/geo/query/acc.cgi?acc=GSE39144), and GSE9709²⁹) of hESCs and/or hiPSCs were analyzed against various sets of differentiated cell types (e.g., foreskin fibroblast, neuronal differentiated cells from hESCs/hiPSCs, or endothelial cells). The microarray dataset was analyzed using GEO2R (found on the world wide web at https://www.ncbi.nlm.nih.gov/geo/geo2r/) to identify acetylation-modulating factor(s) whose expression is significantly different in hPSCs compared to their differentiated counterparts ¹¹. Of 40,000-50,000 primers, corresponding to mRNA transcripts, only the top 20% mRNA transcripts were selected as a cut-off range to validate significance, based on p values. Each gene expression in a given database was further monitored across multiple groups of hPSCs to determine gene expression changes. It was first determined if the expression of any acetyl transferase is consistently altered in hPSCs, but failed to find any in all five meta-analysis studies (Table 4). All known deacetylases were next analyzed; 11 HDACs (belonging to HDAC I, II, and IV) and 7 SIRTs (belonging to HDAC III). Remarkably, SIRT2 was found to be uniquely and consistently downregulated in all five independent meta-analyses using multiple sets of hPSCs (FIGS. 14A and 14B and Table 5). In addition, SIRT1 is upregulated in hPSCs in four meta-analyses. Furthermore, using another web-based database analysis tool (found on the world wide web at http://nextbio.com), downregulation of SIRT2 gene expression and upregulation of SIRT1 were observed without any exception in 25 hESCs compared to 15 human somatic cells (FIG. 1B and Table 6). In contrast, expression levels of other sirtuins (SIRT3-7) were variable between hESC lines and somatic cells (FIGS. 14C-14G). Without wishing to be bound by a particular theory, these findings prompted the hypothesis that altered acetylation of metabolic enzymes by SIRT1 and/or 2 plays a critical role(s) in metabolic reprogramming and pluripotent stem cell functions. To test this, their gene expression was examined during somatic reprogramming and in vitro differentiation. As shown in FIGS. 1C and 1D, SIRT2 expression (both mRNA and protein level) was prominently downregulated while SIRT1 expression was upregulated in hPSCs compared to fibroblasts, showing that induced pluripotency accompanies SIRT1 induction and SIRT2 suppression. In contrast, during spontaneous in vitro differentiation, SIRT2 expression was highly upregulated while SIRT1 expression was downregulated along with pluripotency markers Oct4 and Sox2 (FIG. 1E). In addition, SIRT2 was robustly up-regulated during lineage-specific in vitro differentiation of hESCs into midbrain dopamine neuron (FIGS. 1G and 1I), as evidenced by dramatic increases in expression of Tuj1 (encoded from TUBB3: Tubulin beta 3), tyrosine hydroxylase (TH), and transcription factor Lmxlb (FIGS. 1F and 1G), which was accompanied by a robust decrease in the expression of SIRT1, Oct4 and Nanog (FIGS. 1H and 1I).

Functional Effects of SIRT2 Knockdown in hPSCs

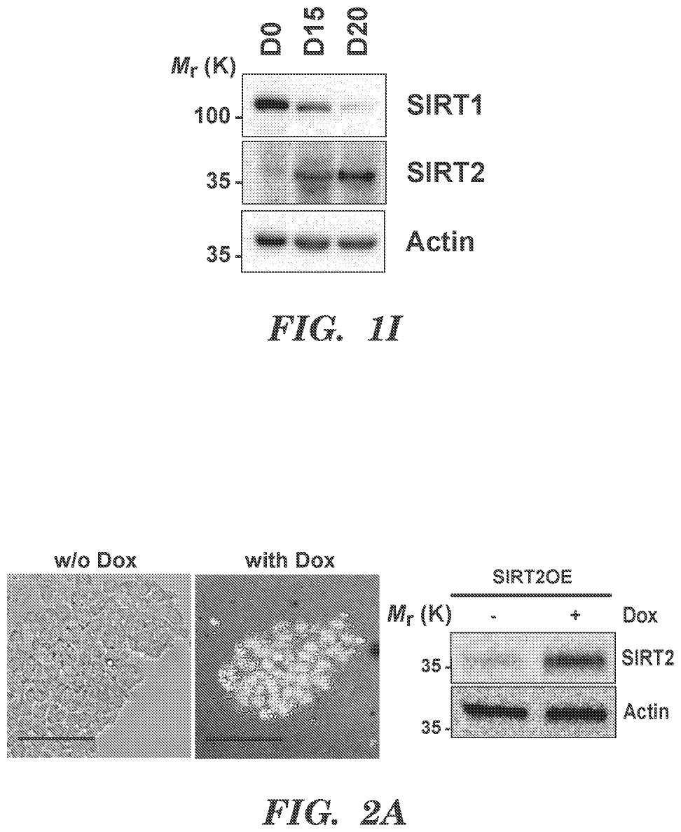

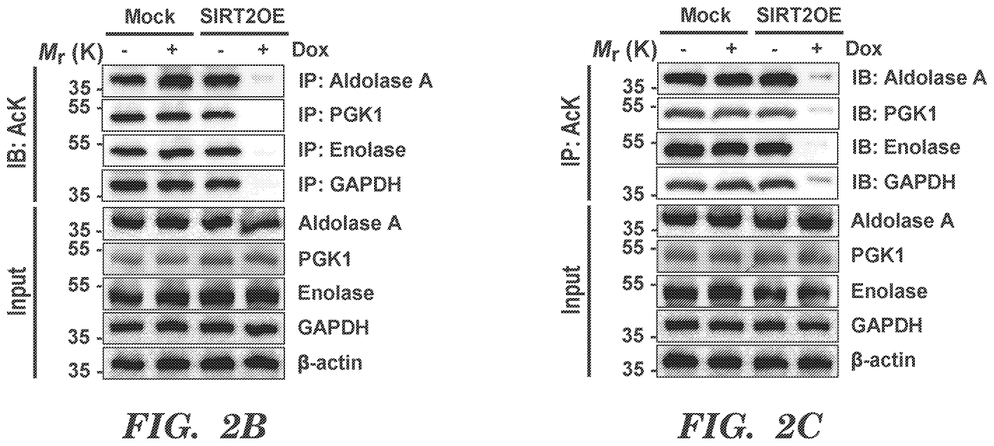

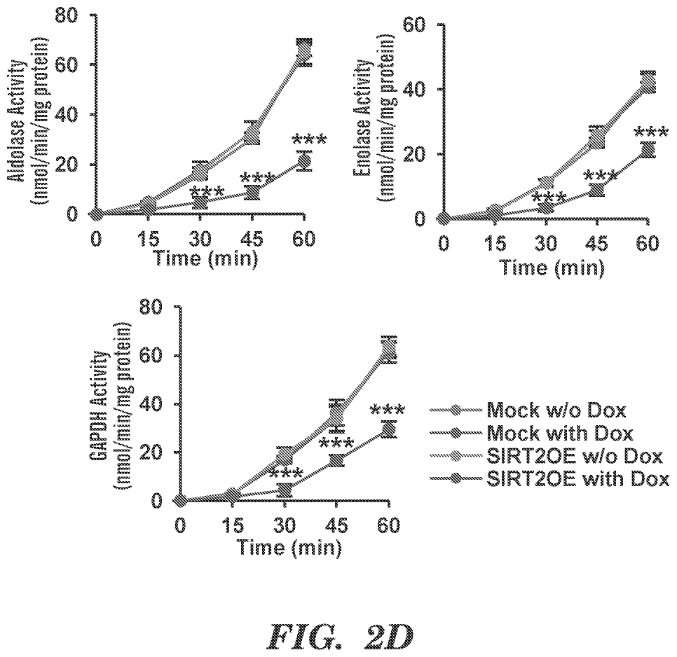

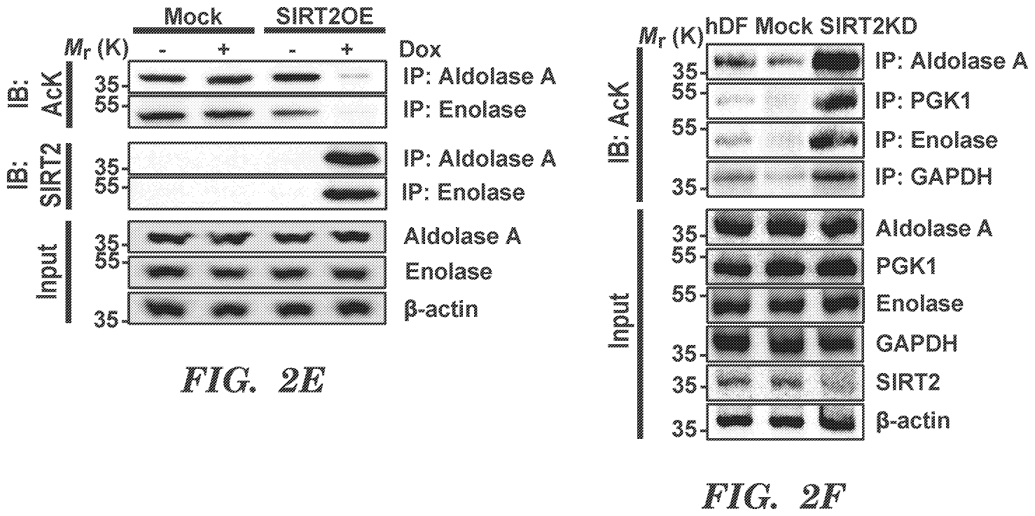

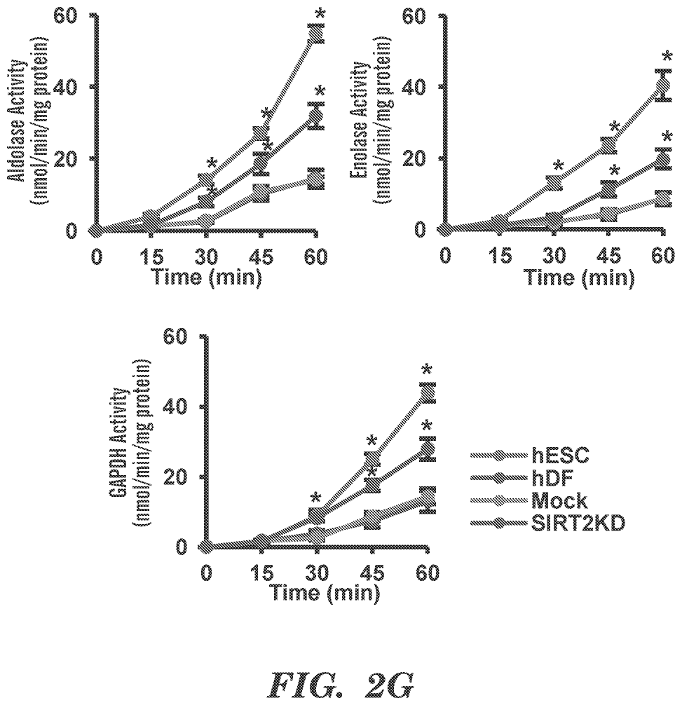

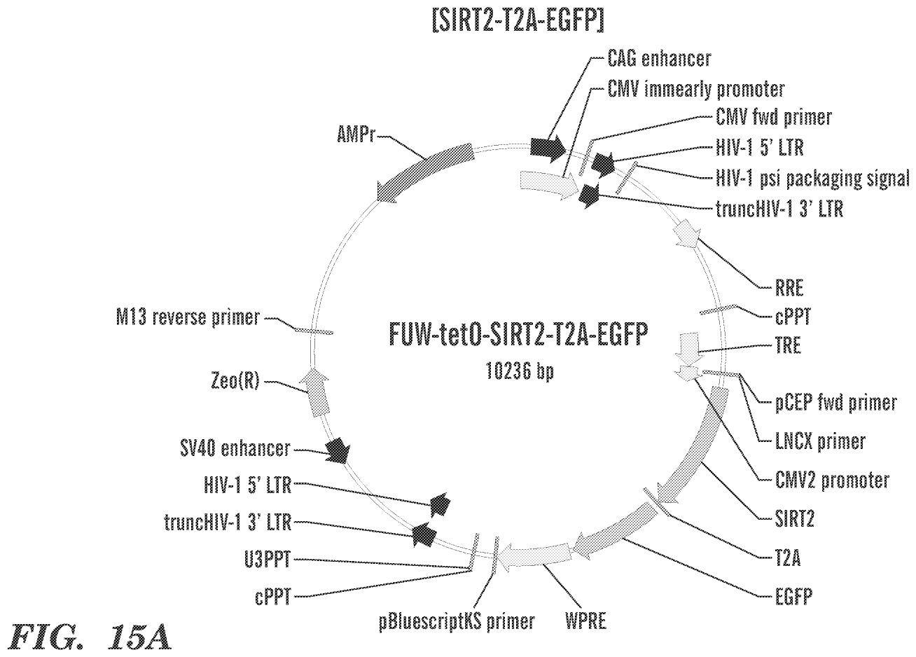

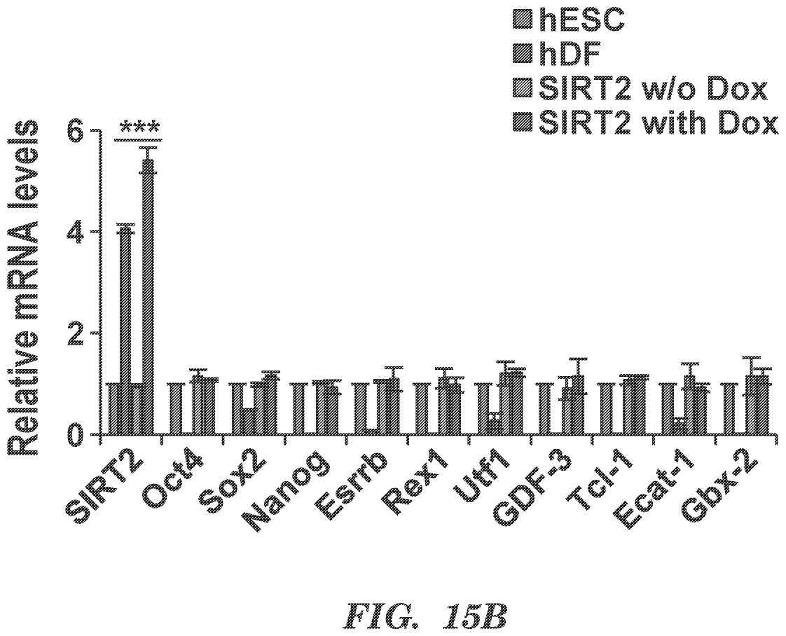

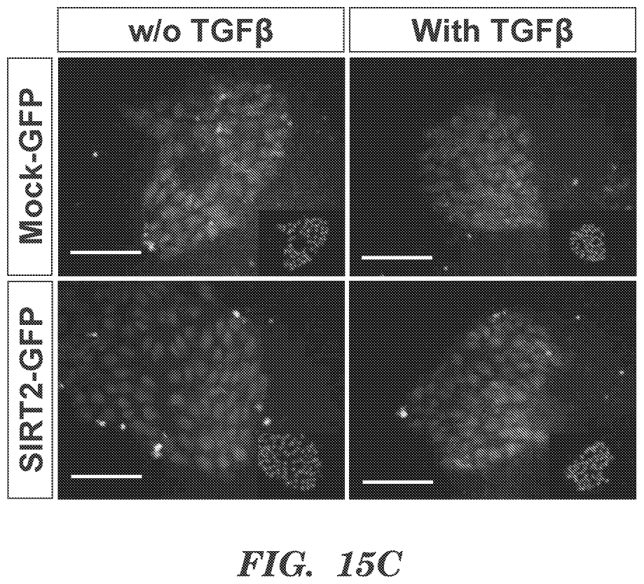

Because glycolytic enzymes (e.g., aldolase (ALDOA), glyceraldehyde-3-phosphate dehydrogenase (GAPDH), phosphoglycerate kinase (PGK1), enolase (ENO1), and pyruvate kinase) are highly acetylated and the deacetylase SIRT2 is robustly downregulated in hESCs, it was hypothesized, without wishing to be bound by theory, that SIRT2 downregulation is responsible for their hyperacetylation, directly contributing to the Warburg-like effect. To address this, stable hESC lines were first generated in which expression of SIRT2 and EGFP can be induced by doxycycline (Dox) using a lentiviral vector (FIGS. 2A and 15A). Under normal hESC culture condition, this hESC line (H9-SIRT2OE) exhibited the same morphology as wild type hESCs (H9) with or without Dox treatment (FIG. 2A). However, their self-renewal and pluripotent differentiation function were altered, as described herein below. To investigate the effect of altered SIRT2 expression on acetylation and enzymatic activities of these glycolytic proteins, each glycolytic protein was pulled down by immunoprecipitation with their respective specific antibody and western blotting was performed using an anti-acetyl-Lys antibody. As shown in FIG. 2B, forced expression of SIRT2 in hESCs prominently deacetylated all four enzymes tested (aldolase, PGK1, enolase, and GAPDH). The same pattern was observed when proteins were first immunoprecipitated using acetyl-Lys antibody followed by western blotting using specific antibodies against each protein (FIG. 2C). In contrast to the altered acetylation levels of these enzymes, expression levels of their total proteins (see Input; FIGS. 2B and 2C) and mRNAs (FIG. 15B) were unchanged. PKM1 and 2 could not be analyzed here due to the lack of specific antibodies that can distinguish these isoforms. Whether altered acetylation affects their enzymatic activities was next assessed. As shown in FIG. 2D, deacetylation of glycolytic enzymes by SIRT2 overexpression (OE) in hESCs caused a significant decrease of enzymatic activities for all three enzymes tested (aldolase, enolase, and GAPDH) while the total proteins were unchanged (FIGS. 2B and 2C). Remarkably, SIRT2 bound to aldolase and enolase (FIG. 2E), but not to PGK1 or GAPDH (data not shown), likely due to their weaker interaction and/or to the lower affinity of the antibodies used herein.

Next, the effect of SIRT2 knockdown (KD) on glycolytic enzymes in hDFs was investigated using lentiviral SIRT2 shRNAs. Each protein was pulled down using specific antibody and detected by western blotting using anti-acetyl-Lys antibody. Acetylation levels of aldolase, enolase, PGK1 and GAPDH were substantially increased in SIRT2 KD fibroblasts, compared to original fibroblasts or mock control, while the expression levels of their total proteins were similar (FIG. 2F). Furthermore, their enzymatic activities were significantly increased, indicating a direct correlation between their acetylation levels and activities (FIG. 2G). In contrast to SIRT2, SIRT1 OE in hDFs affected neither acetylation levels nor activities of these enzymes (data not shown).

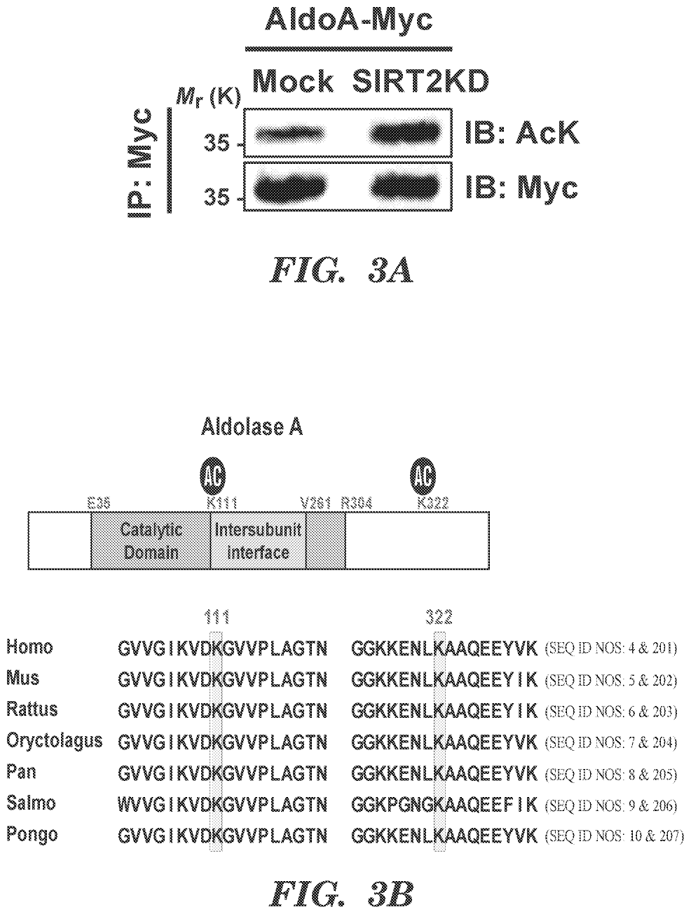

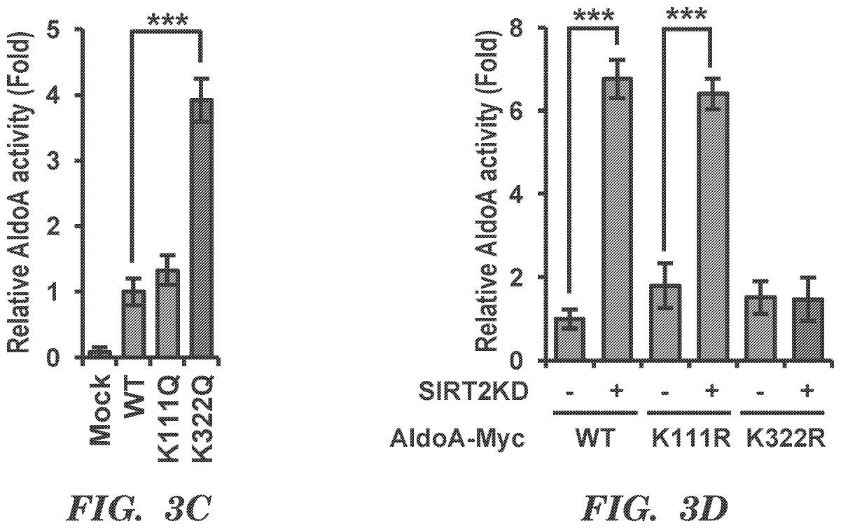

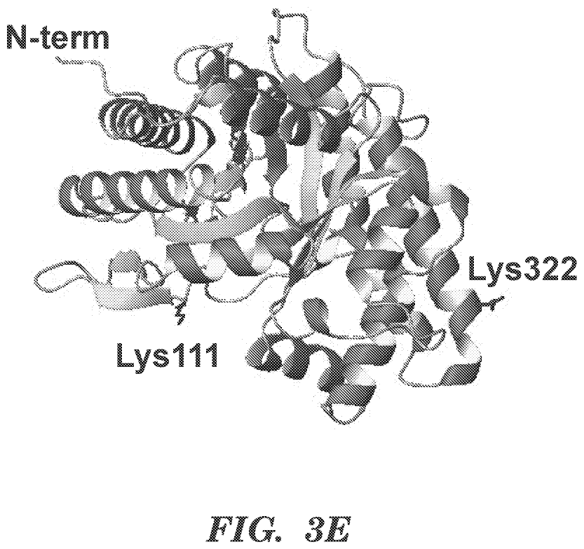

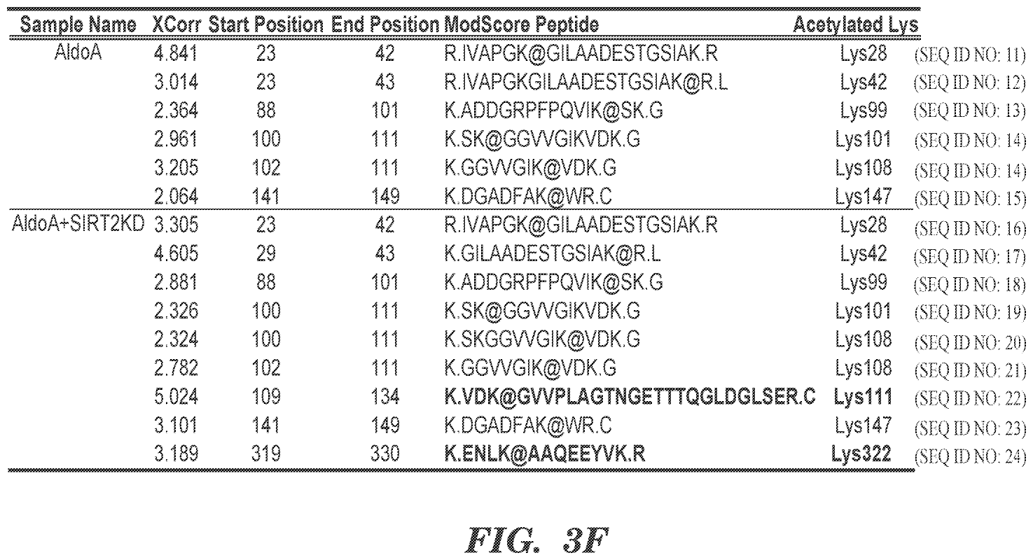

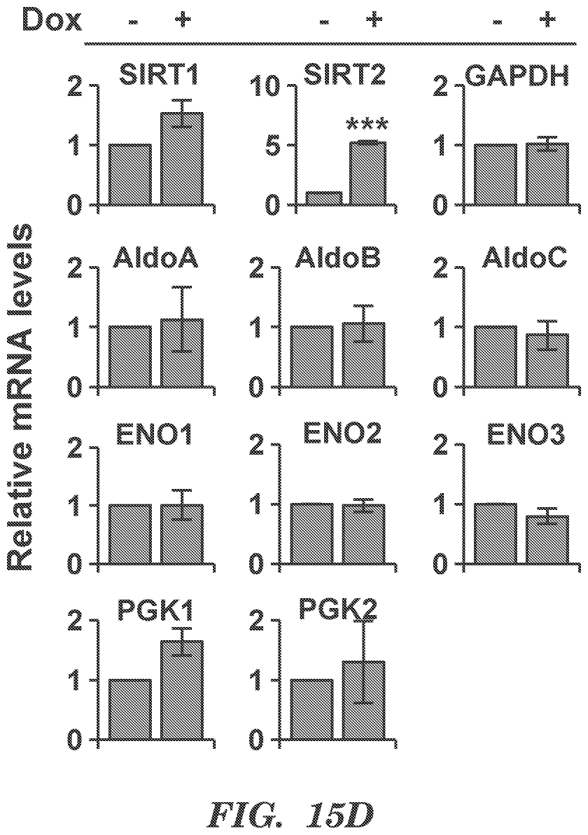

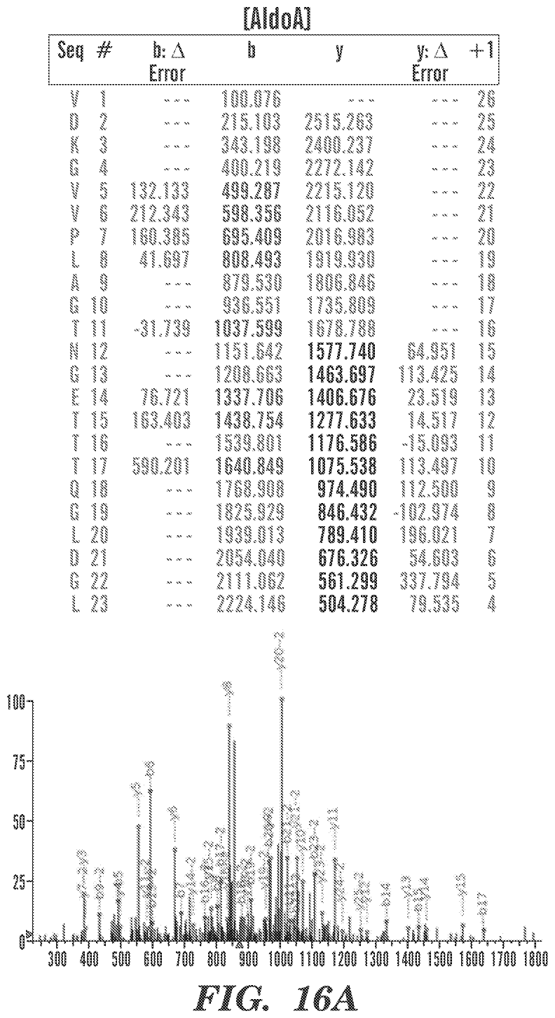

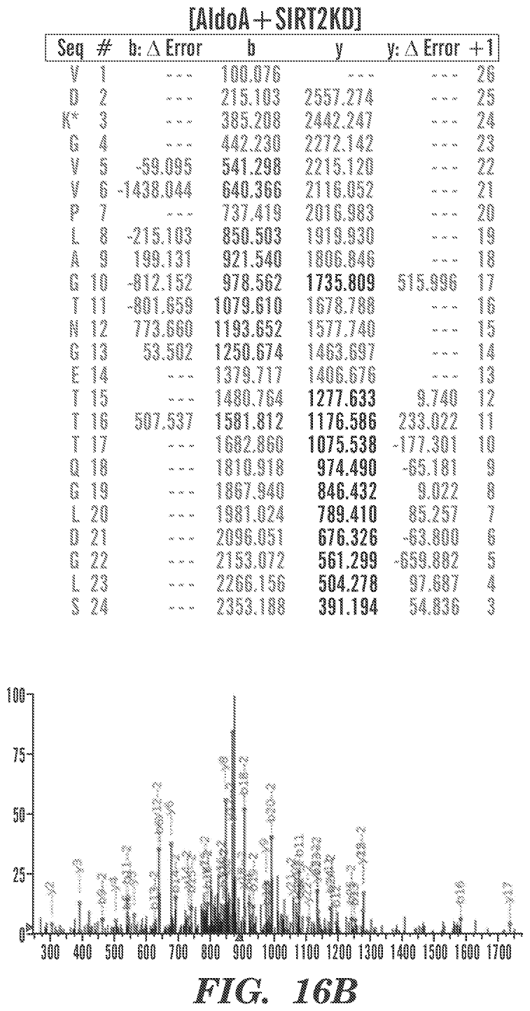

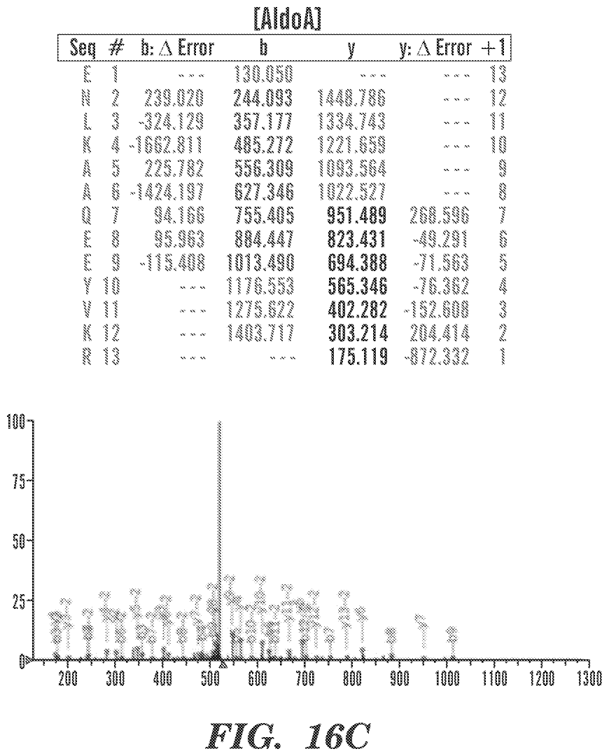

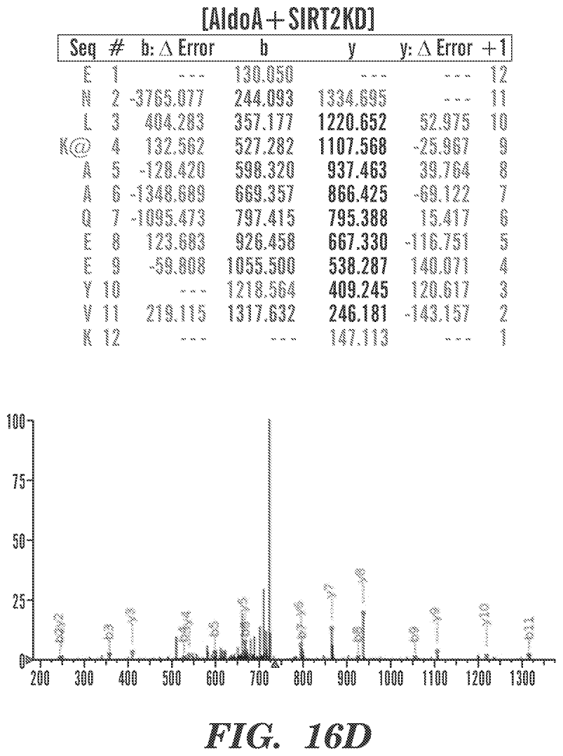

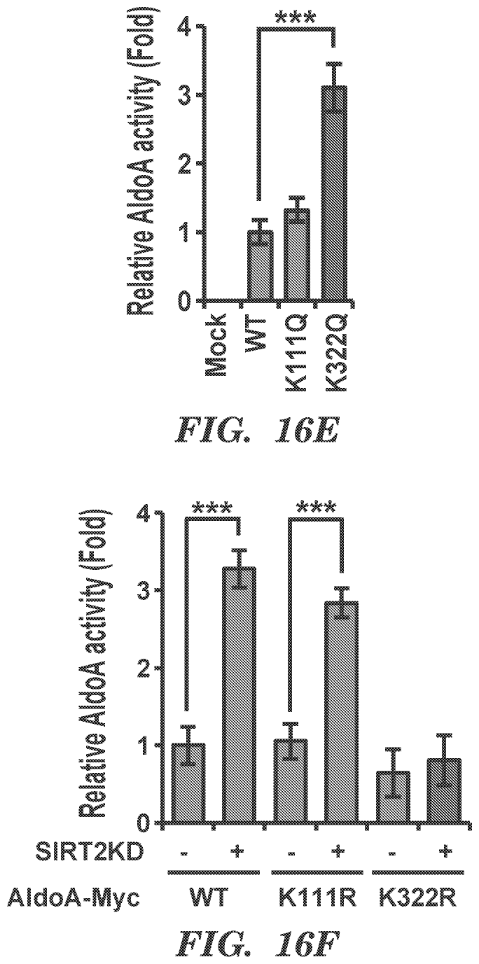

The findings presented herein are surprising because acetylation is generally known to inhibit most metabolic enzymes³⁴. Thus, it was sought to identify specific lysine residues and analyzed the functional effects of their deacetylation by SIRT2, using aldolase (AldoA) as an example. Using LTQ-Orbitrap mass spectrometry, a total of 6 and 8 Lys residues are highly acetylated in mock- and SIRT2 KD cells, respectively, were found (FIGS. 3A and 3B). Interestingly, 2 residues (i.e., K111 and K322) are enriched in SIRT2 KD cells, but not in control cells. Representative spectra of acetylated peptide at K111 and 322 by LC-MS/MS analysis are shown in FIGS. 16A, 16B, 16C, and 16D, respectively. Acetylated and non-acetylated forms of AldoA peptides were well separated and the acetylated form of AldoA was shown 42 higher m/z value due to the acetyl groups. According to protein blast searching (found on the world wide web at http://blast.ncbi.nlm.nih.gov/Blast.cgi), the K111, but not the K322, residue belongs to catalytic domain/intersubunit interface (FIG. 3C)³⁵. Thus, the K322 residue represents an as-yet-unidentified domain. In addition, sequence alignment of AldoA showed that K111 and K322 are highly conserved among diverse species (FIG. 3C). To further determine whether K111 and/or K322 represent SIRT2 target sites and play a role for regulating AldoA, each of them were mutated to glutamine (Q; acetylated mimetic) or arginine (R; deacetylated mimetic) and their activity was examined. The mutation of K322, but not K111 to Q, was found to robustly increase the catalytic activity of AldoA compared to wild type in both hDFs and 293T cells (FIGS. 3D and 16E). Moreover, SIRT2 KD prominently activated wild-type AldoA and K111R mutant, but not K322R mutant (FIGS. 3E and 16F), demonstrating that K322 is an important site of acetylation and that its deacetylation by SIRT2 significantly downregulates its activity. This result further corroborates the findings that SIRT2 levels regulate acetylation and enzymatic activities of aldolase (FIGS. 2B-2G). Notably, AldoA structure model showed that K322 is exposed to the outside surface of AldoA, indicating its availability to bind to SIRT2 (crystal structure model of human Aldolase A, Protein Data Bank code: 1 ALD) (FIG. 3F)³⁶. Taken together, these finding indicate that SIRT2 directly controls the acetylation levels and enzymatic activities of glycolytic enzymes and contributes to metabolic reprogramming.

SIRT2 Expression Levels Influence Metabolism, Cell Survival, and Pluripotent Differentiation Functions of hPSCs

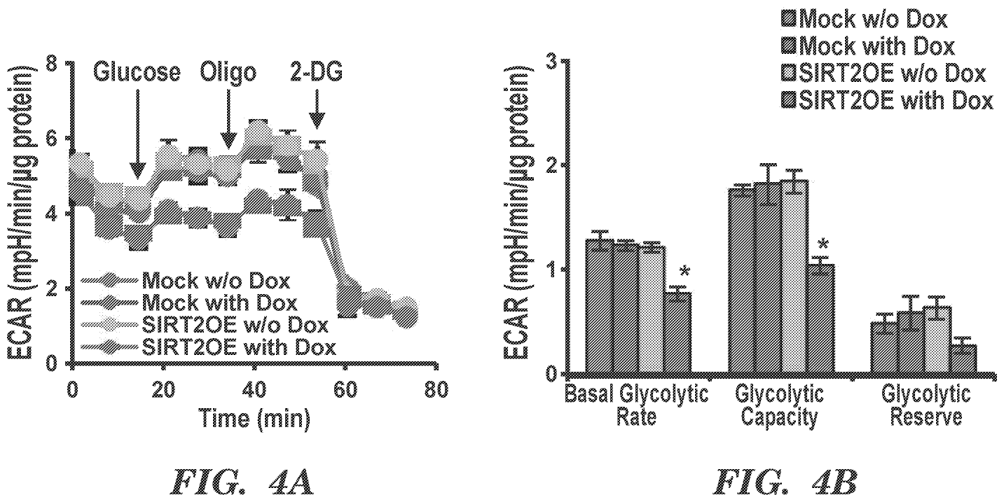

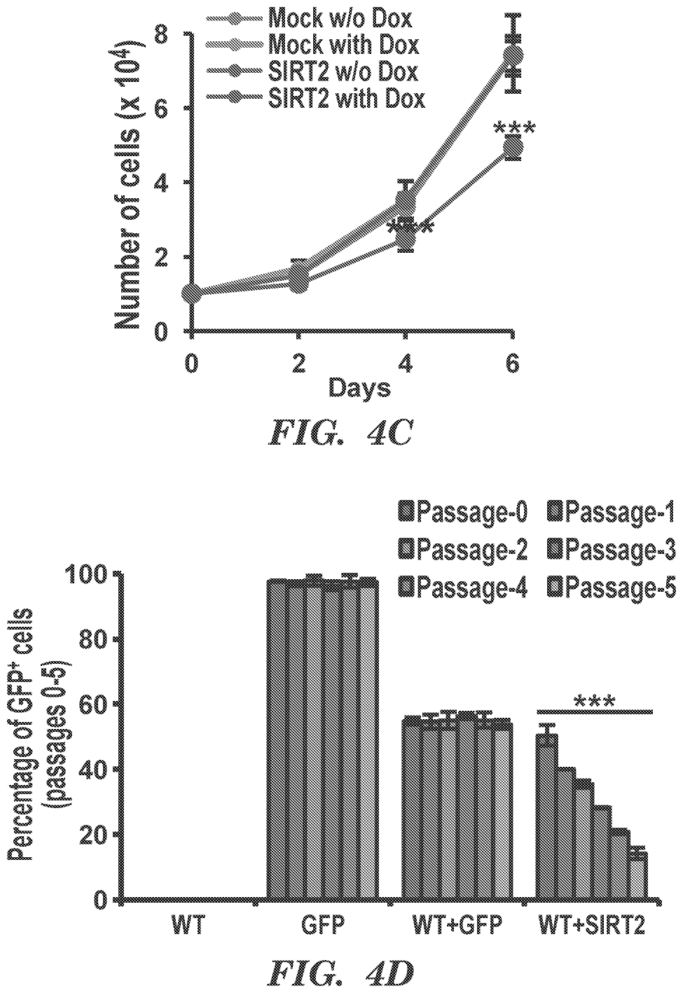

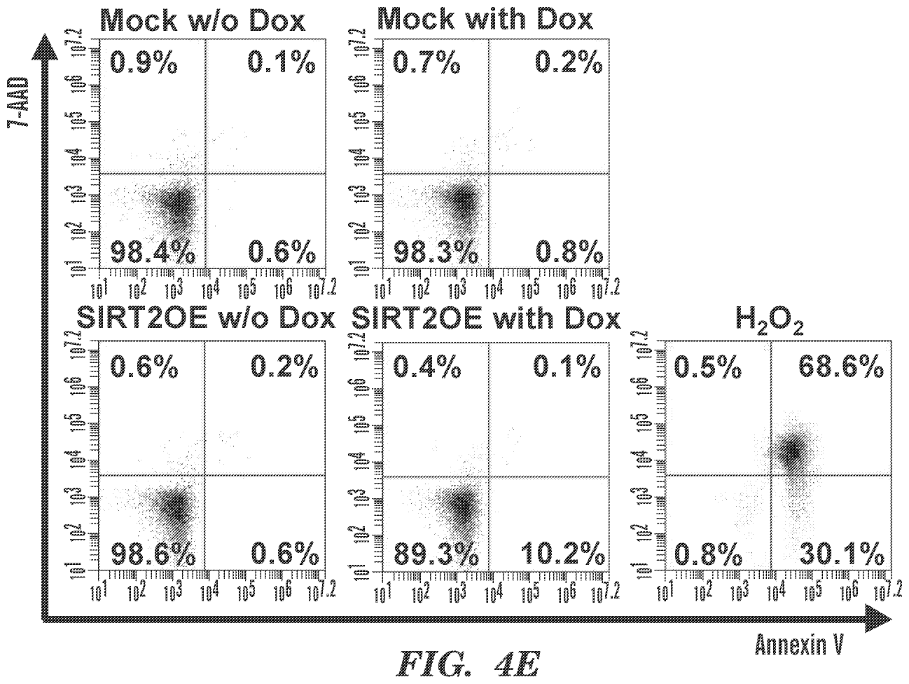

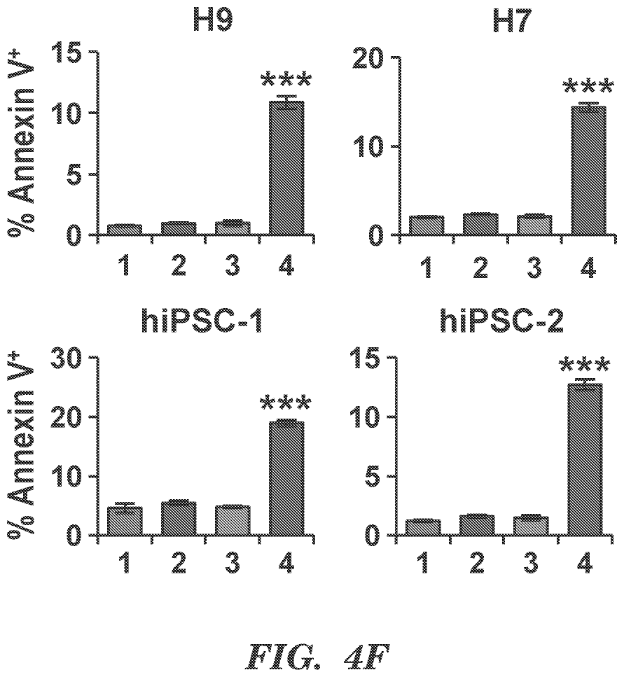

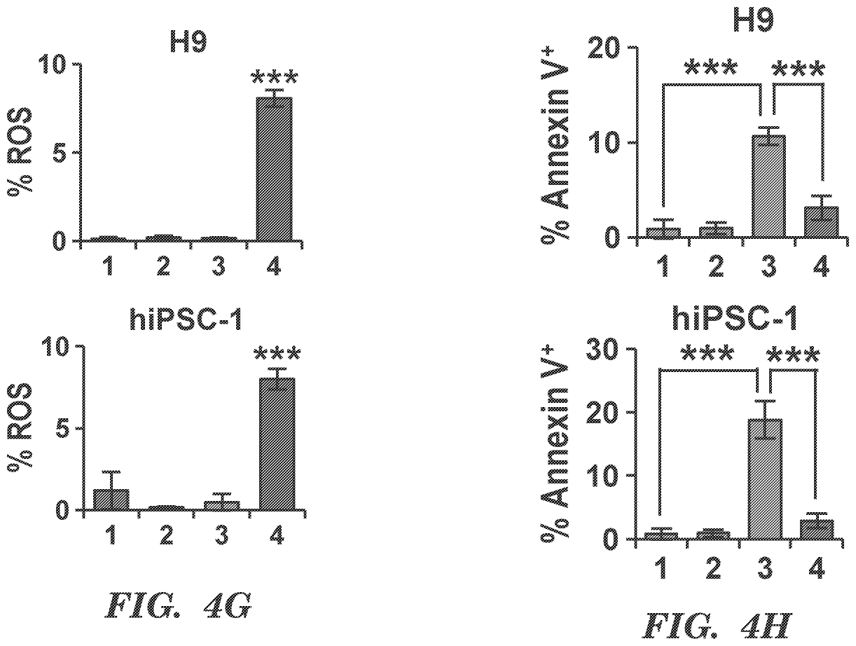

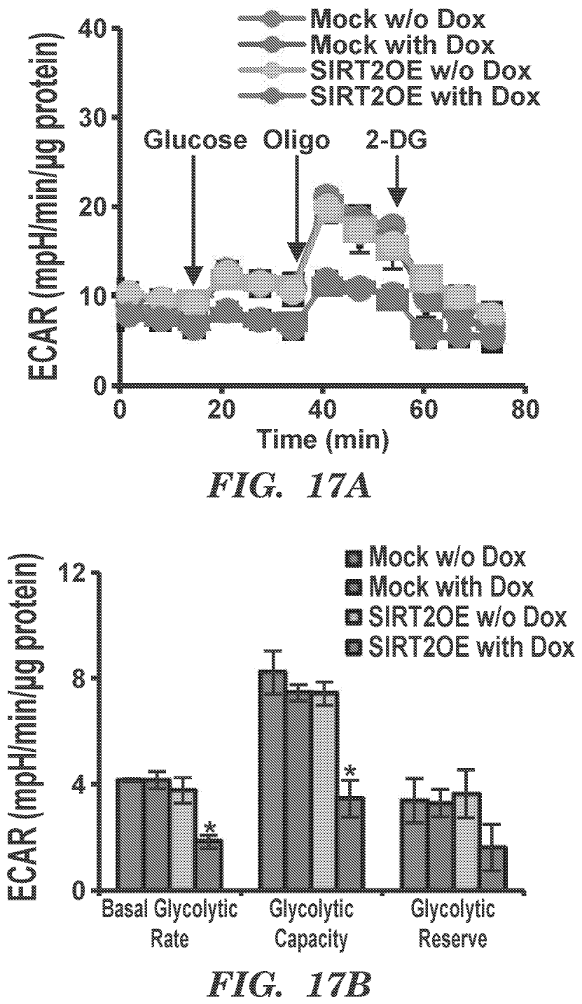

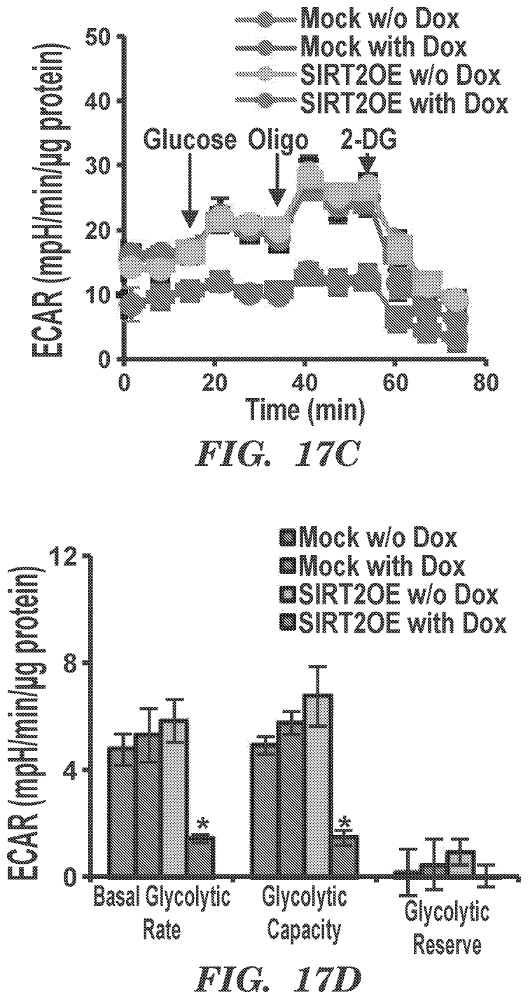

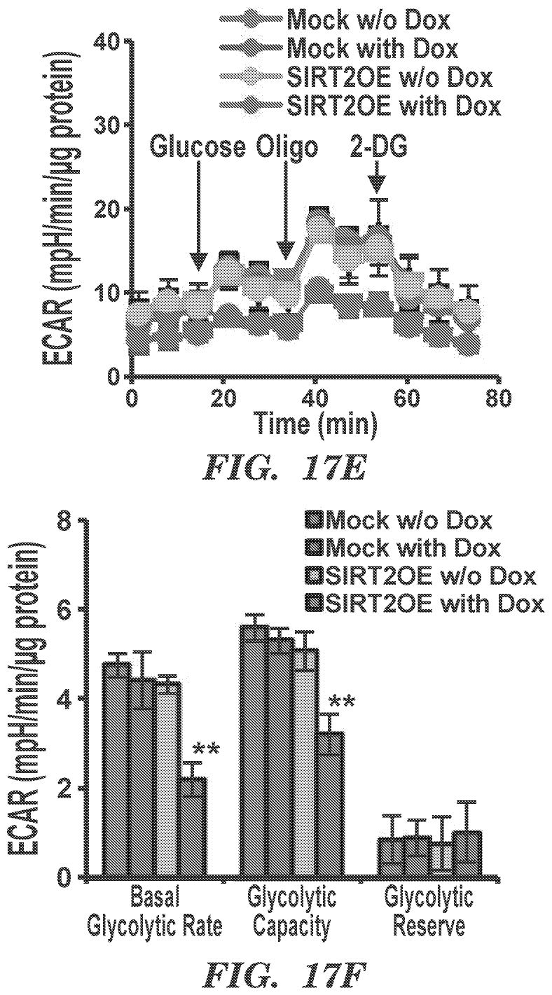

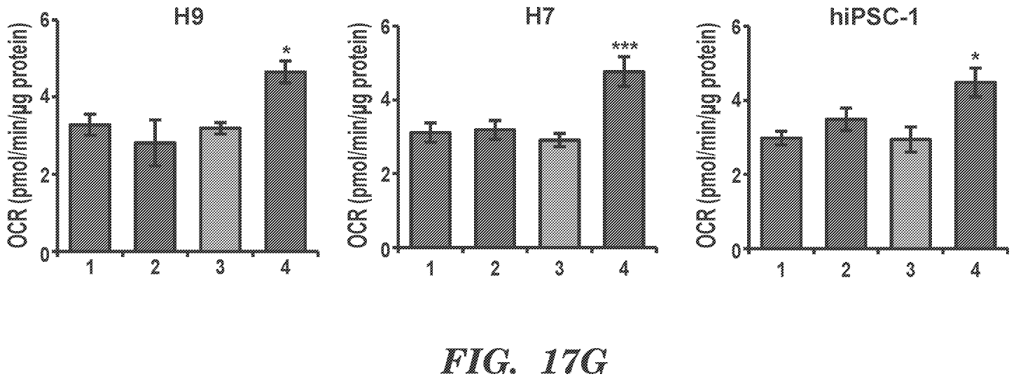

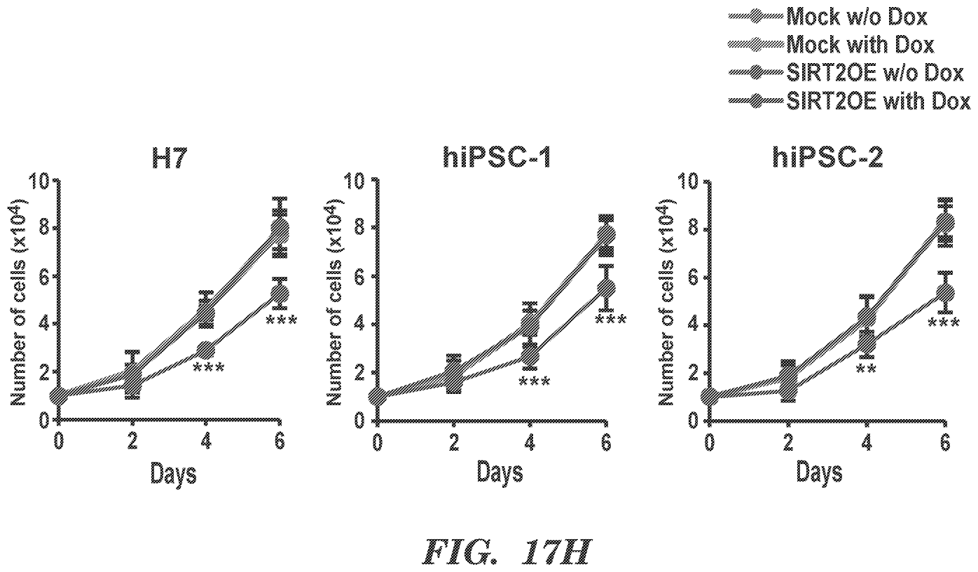

It was next determined if altered SIRT2 levels directly influence glycolytic metabolism in hPSCs by measuring extracellular acidification rate (ECAR)²⁸. Indeed, Dox-induced SIRT2 OE in hESC cells resulted in a reduction of ECAR, basal glycolytic rate (0.77±0.07 versus 1.21±0.04 mpH/min/μg protein) and glycolytic capacity (1.04±0.08 versus 1.84±0.11 mpH/min/vg protein), compared to control cells (FIGS. 4A and 4B). Furthermore, OCR levels were increased by SIRT2 OE compared to control cells (FIG. 17G). The same pattern was observed with H7 hESCs and two independent iPSC lines (the iPSC line described above (hiPSC-1) and the iPS-DF19-9-11T line from the WiCell Institute (hiPSC-2)) (FIGS. 17A-17G). Interestingly, this Dox-induced SIRT2 OE did not change expression levels of pluripotent markers (e.g., Oct4, Nanog, Esrrb, and Rex1) (FIG. 15C) or the morphology of hESCs (FIG. 2A) under nondifferentiating condition. However, the proliferation rate of SIRT2-overexpressing hPSCs was significantly reduced compared to control cells (FIGS. 4A and 17H). a fluorescence-based competition assay was next performed^{37, 38}. When wild-type H9 hESCs (WT) were mixed at a ratio of 1:1 with GFP-overexpressing H9 cells (GFP), the ratios of GFP⁺/total cells remained 50% at each passage up-to 5 passages. In contrast, when WT cells were mixed at a ratio of 1:1 with GFP-overexpressing (GFP) and SIRT2-overexpressing H9 cells (SIRT2), the ratio of GFP+SIRT2-overexpressing cells progressively decreased (FIG. 4D). Since this compromised proliferation/self-renewal capacity can be caused by altered self-renewal per se, cellular senescence, and/or cell death, the cell population was next examined for the presence of the earliest marker of apoptosis, Annexin V. Interestingly, it was found that SIRT2 OE significantly increased the population of apoptotic cells in all 4 hPSC lines tested (FIGS. 4E and 3F). In addition, it was found that intracellular levels of reactive oxygen species (ROS) were increased by SIRT2 OE (FIGS. 4G and 4H). Furthermore, SIRT2-induced cell death was rescued by pretreatment with N-acetyl-L-Cysteine (NAC), a potent ROS scavenger, indicating that induced SIRT2 levels can cause ROS-dependent apoptotic cell death, leading to compromised proliferation/self-renewal capacity.

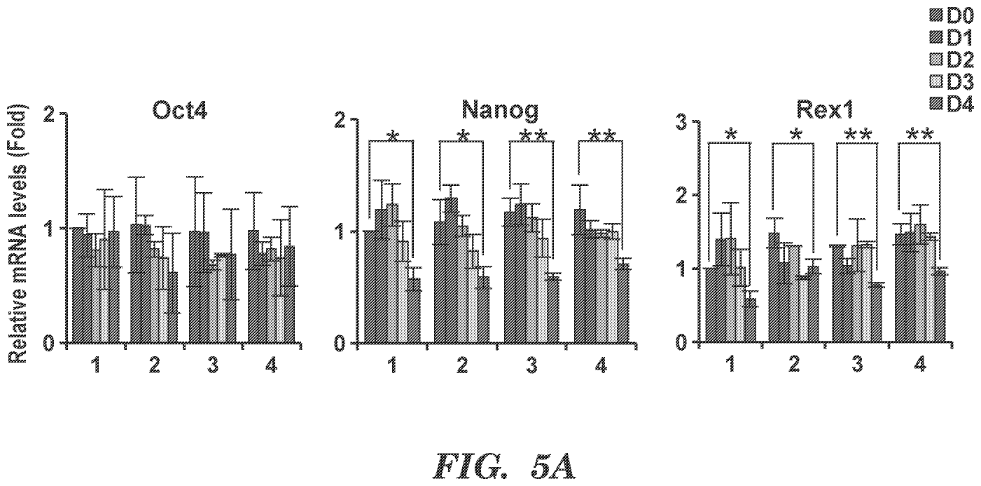

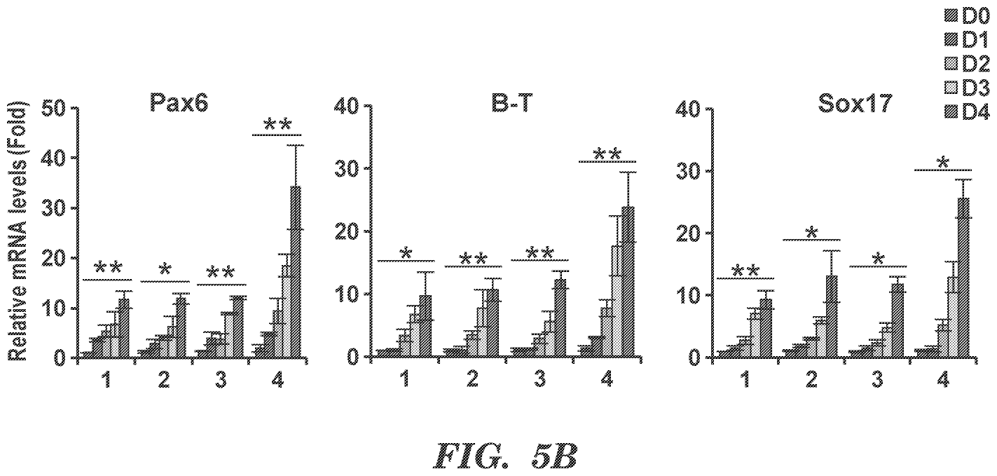

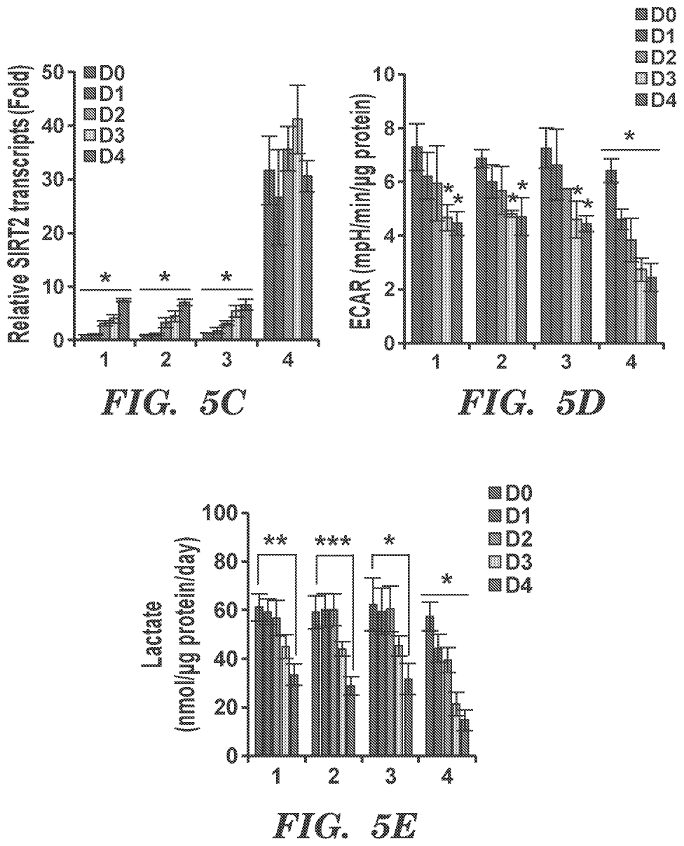

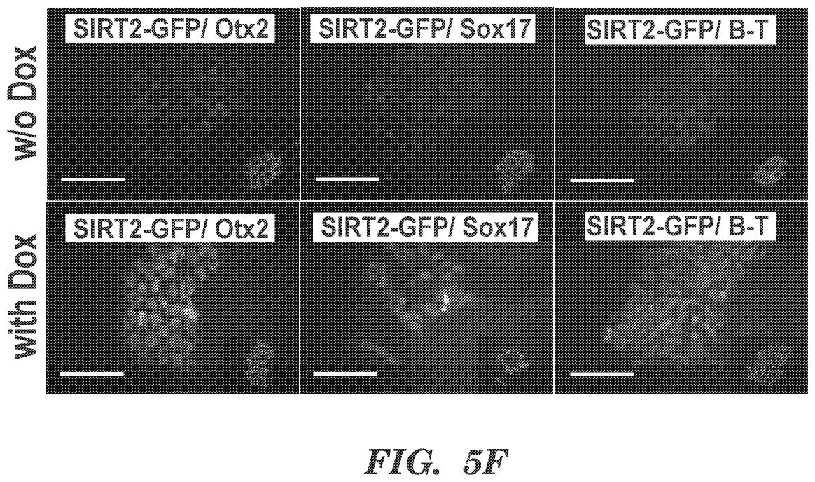

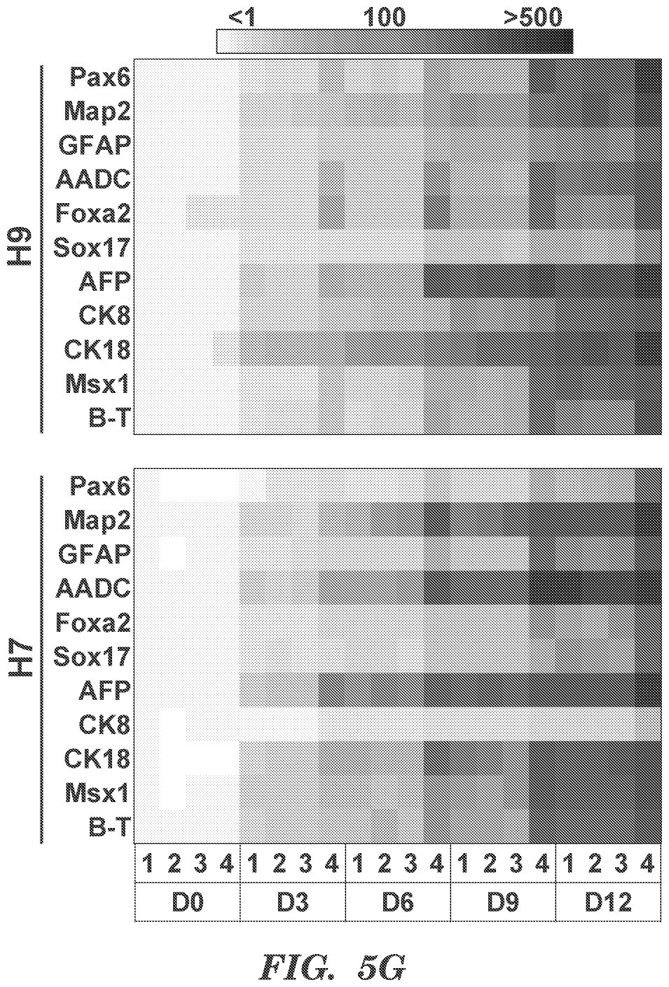

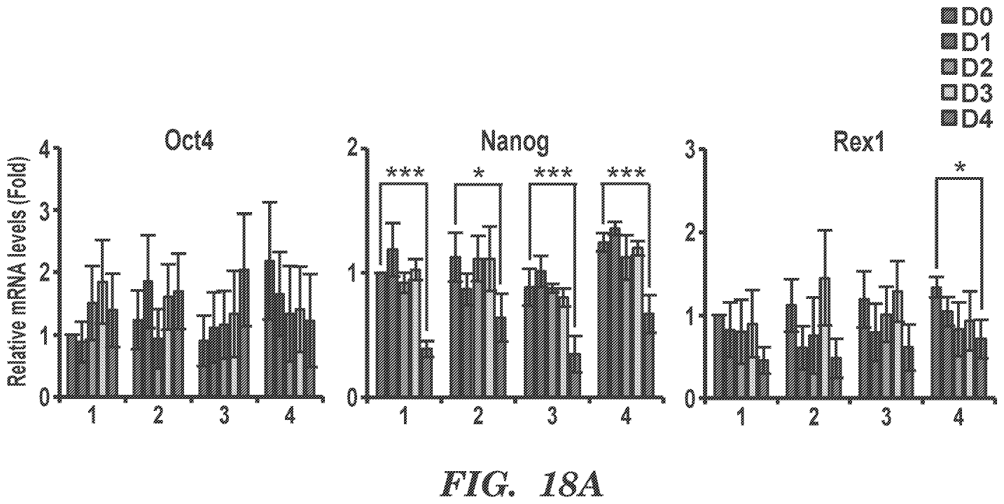

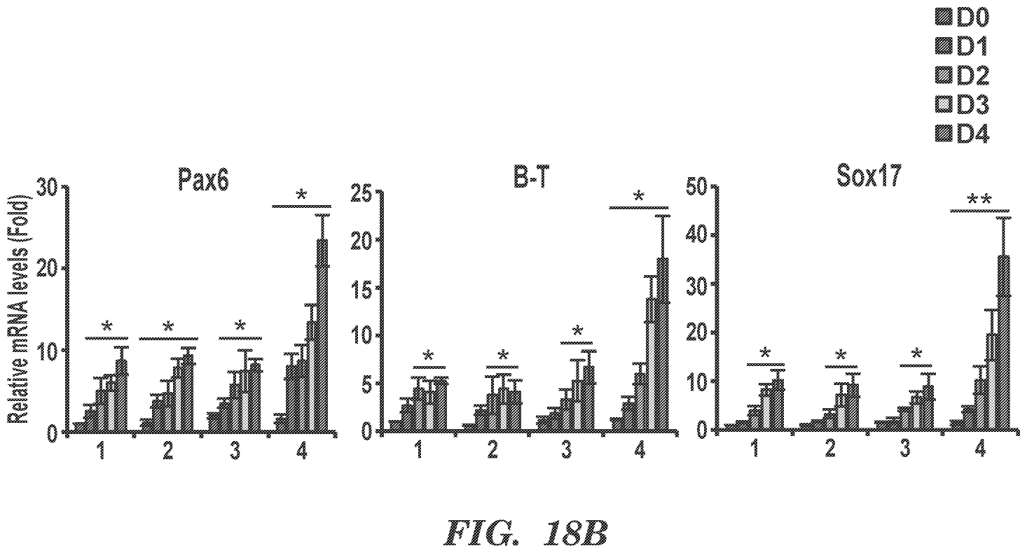

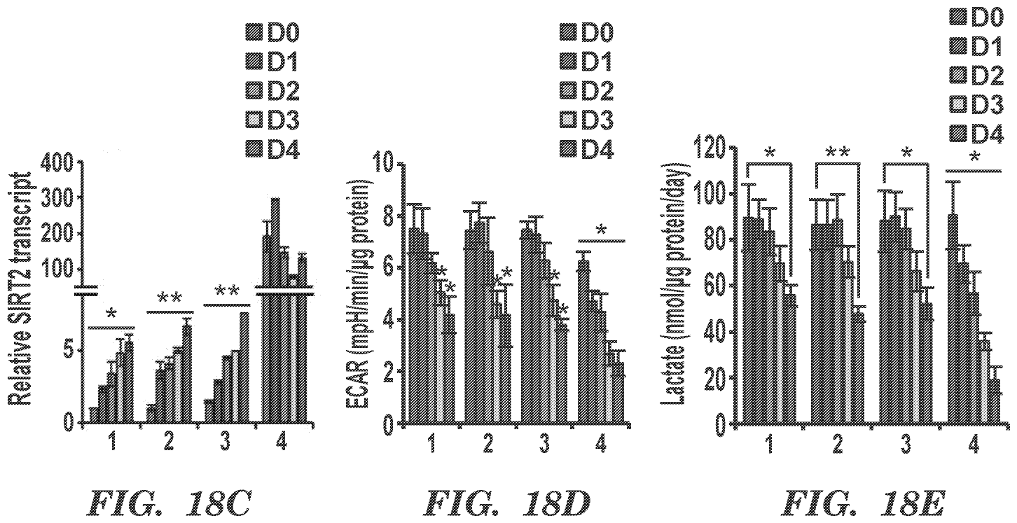

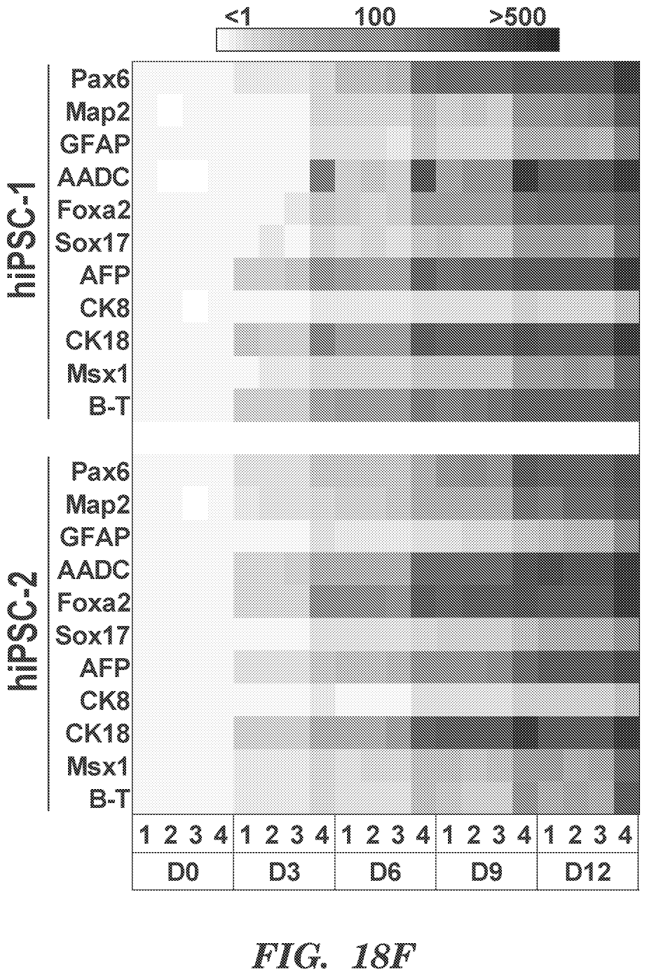

Next, the effect of SIRT2 OE on metabolic reprogramming during the early stage of differentiation was investigated. mRNA expression patterns for pluripotency and lineage-specific early markers were examined. In addition, production of extracellular lactate, a key metabolite of glycolysis, was measured during in vitro differentiation of H9 hESCs. As shown in FIGS. 5A-5C, SIRT2 expression was prominently upregulated within 2 days after differentiation along with early differentiation markers including Pax6, Brachyury (B-T), and Sox17. Furthermore, ECAR levels in hPSCs were decreased as early as 3 days during in vitro differentiation, while lactate production was significantly reduced at day 4 during in vitro differentiation (FIGS. 5D and 5E). Remarkably, Dox-induced SIRT2 OE in H9 hESCs during in vitro differentiation resulted in a significant reduction of ECAR and extracellular lactate production compared to control cells (FIGS. 5D and 5E). The same pattern was observed with the hiPSC-1 line (FIGS. 18A-18E). These findings strongly support the hypothesis that altered SIRT2 expression directly influences metabolic reprogramming during the early differentiation process of hPSCs followed by a significant change of lactate production. To further determine whether SIRT2 expression levels affect the pluripotent differentiation potential of hESCs, mRNA or protein expression patterns for various lineage markers were examined at day 0, 3, 6, 9 or 12 (DO-D12) during spontaneous in vitro differentiation. Strikingly, SIRT2 overexpressing hESCs differentiated more efficiently than WT and H9-SIRT2 without Dox to all three germ layer lineages, as evidenced by staining with antibodies against Otx2 (ectodermal), Sox17 (endodermal), and Brachyury (mesodermal marker) (FIG. 5F). Furthermore, expression levels of diverse lineage marker genes of all three germ layers were markedly increased in SIRT2 OE hESC lines (H9 and H7) as well as hiPSC lines (hiPSC-1 and hiPSC-2) compared to WT and SIRT2 OE without Dox at all time points tested (D3-D12) (FIGS. 5G and 18F). Taken together, results presented herein indicate that SIRT2 levels in hPSCs directly influence energy metabolism and regulate survival and pluripotent differentiation potential of hPSCs.

Expression Levels of SIRT2 Regulate Energy Metabolism in hDFs and Influence the Reprogramming Process

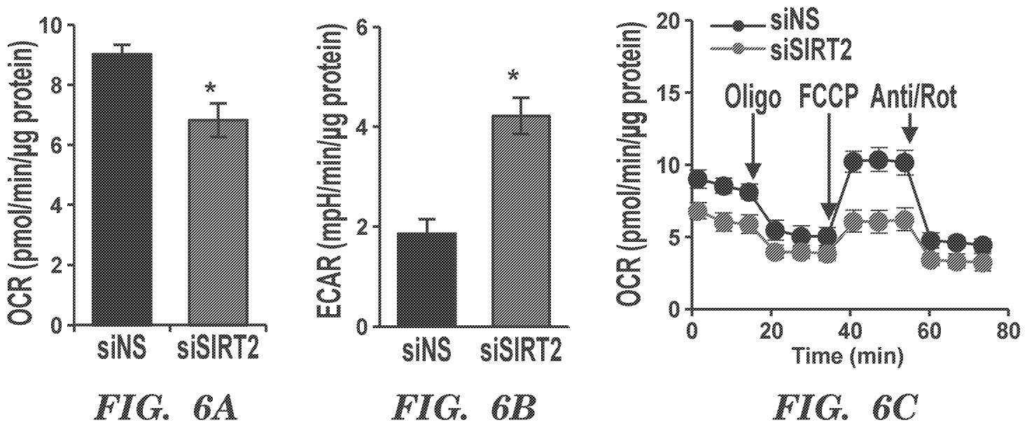

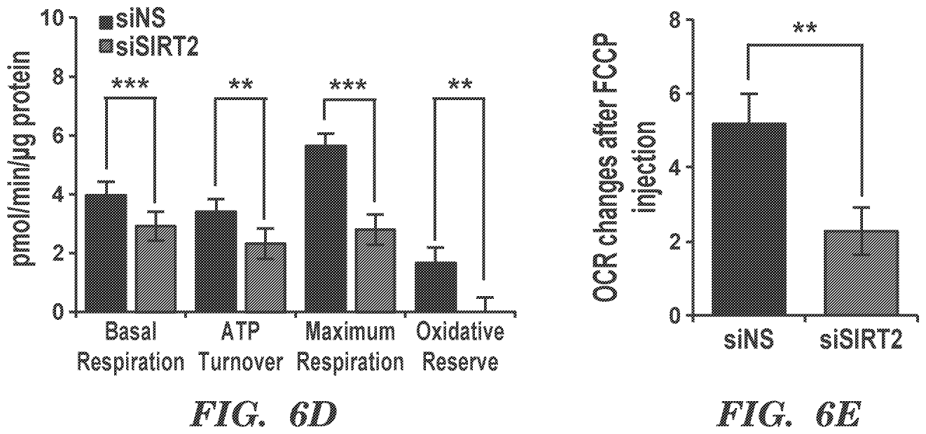

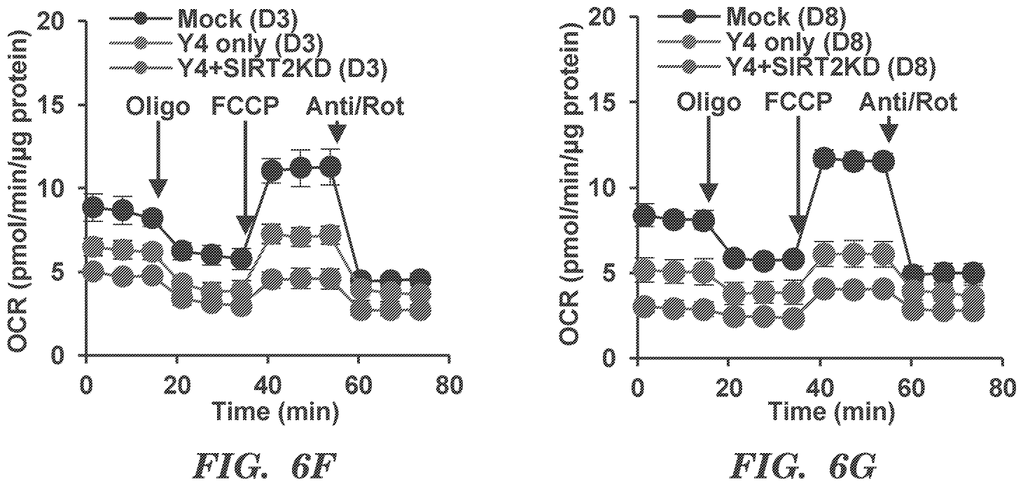

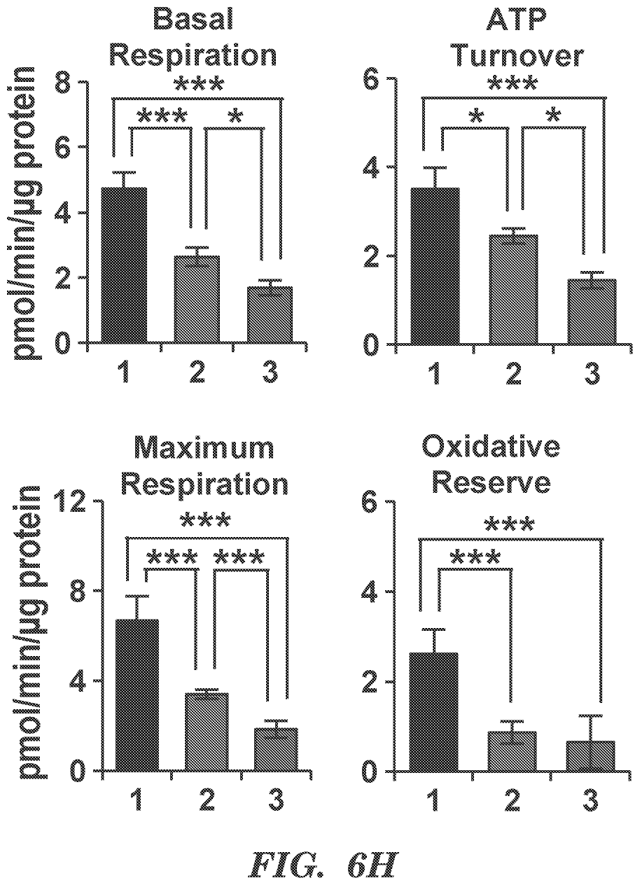

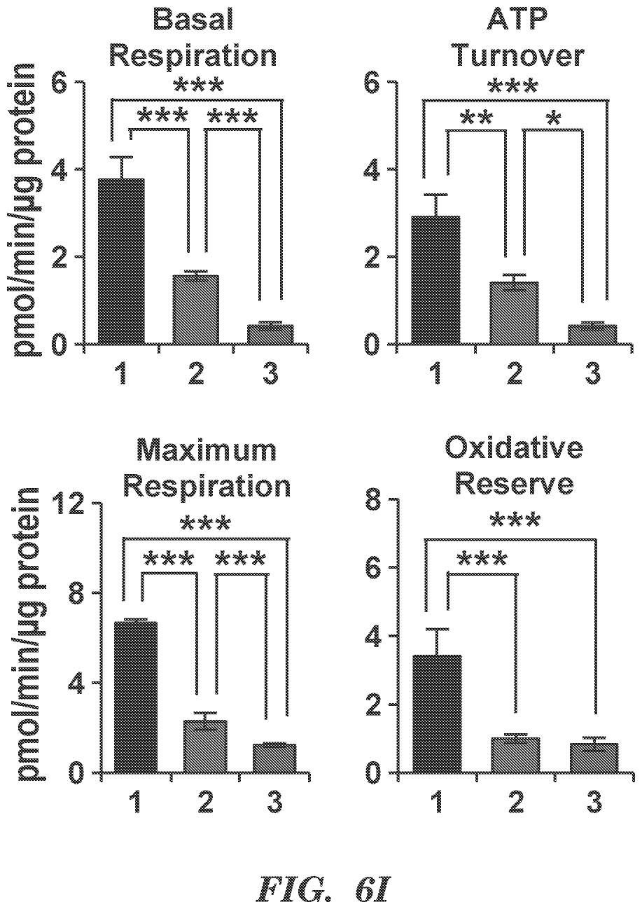

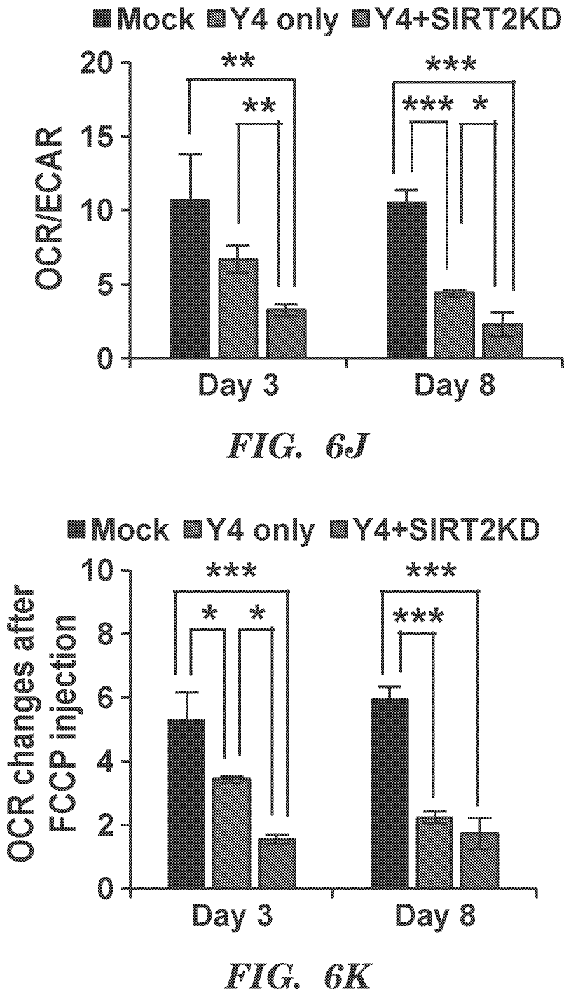

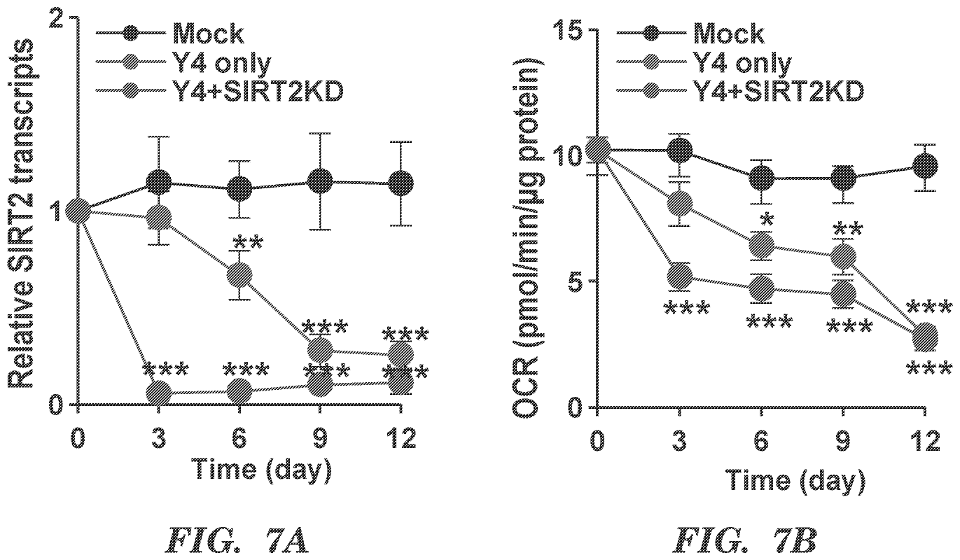

Whether proper regulation of SIRT2 expression is critical for induced pluripotency via regulating metabolic reprogramming was next assessed. To this end, it was first determined whether altered SIRT2 expression induces a metabolic switch in fibroblasts. Indeed, SIRT2 KD in fibroblasts resulted in significant metabolic changes including decreased OCR and increased ECAR compared to control cells (FIGS. 6A and 6B). Furthermore, compared to control, SIRT2 KD cells showed significantly decreased OXPHOS capacity, as evidenced by decreases in basal respiration, ATP turnover, maximum respiration, and oxidative reserve as well as OCR decrease after FCCP treatment (FIGS. 6C-6E). However, SIRT2 KD in fibroblasts by itself was unable to generate any iPSC-like colonies (data not shown). Thus, hDFs were treated with reprogramming factors together with SIRT2 KD. Notably, reprogramming cells with SIRT2 KD showed significantly reduced oxidative metabolism at both day 3 and day 8, compared to control reprogramming cells (FIGS. 6F-6K).

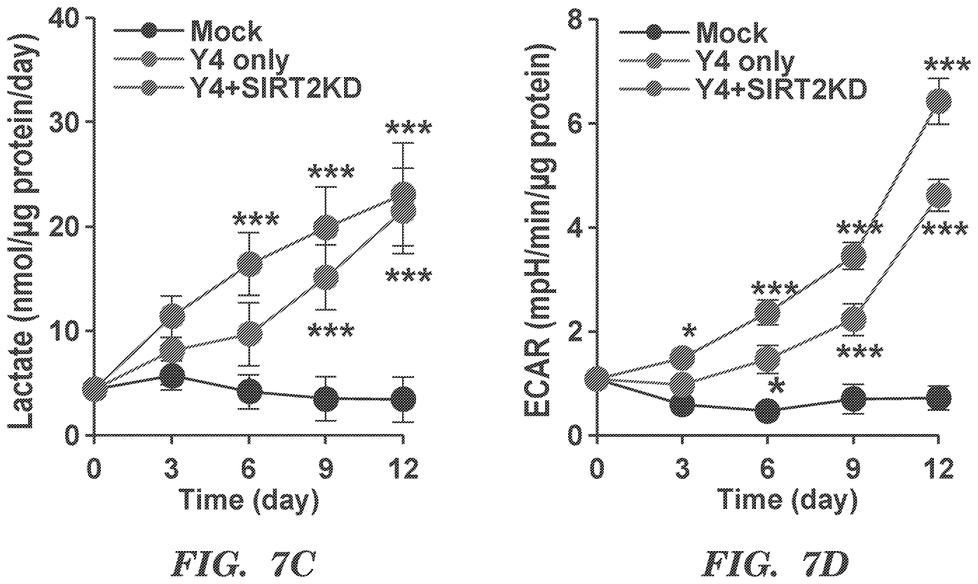

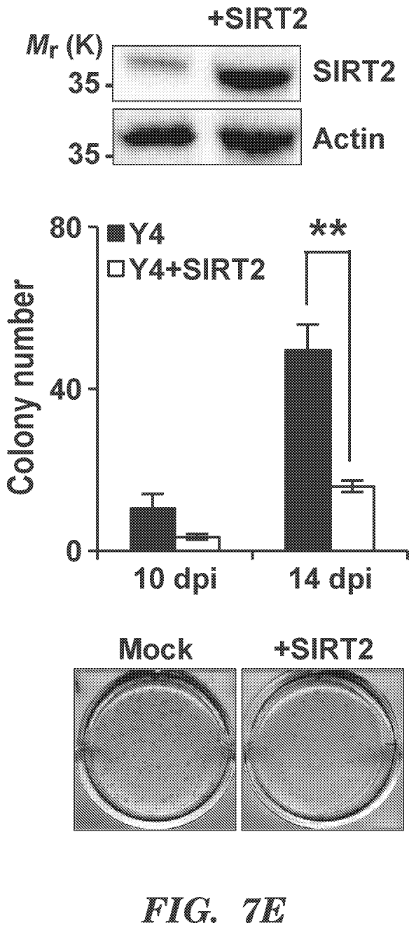

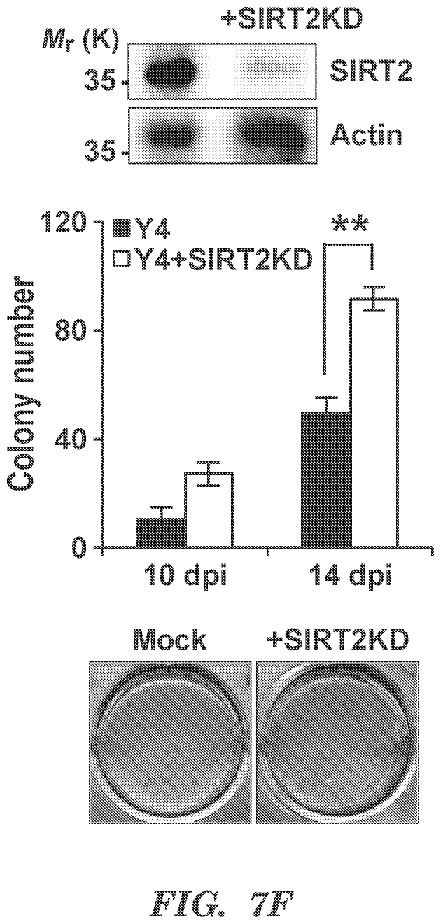

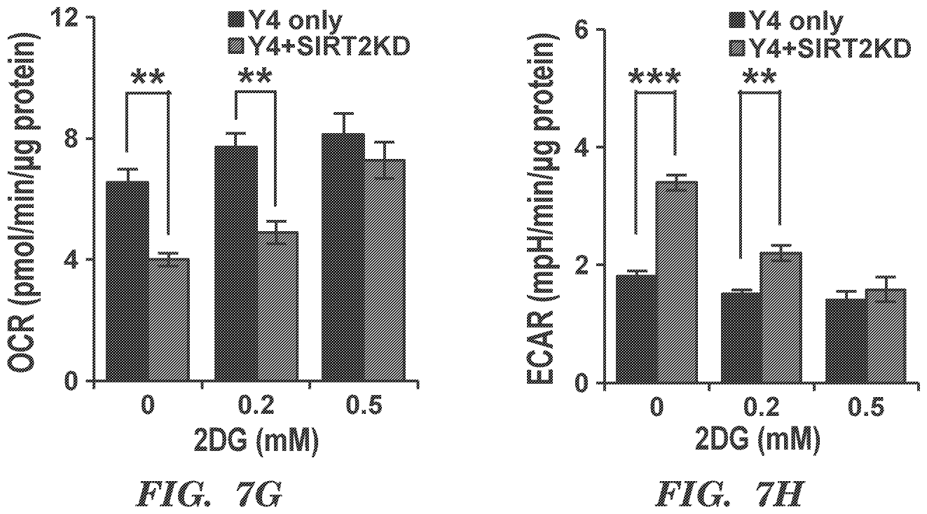

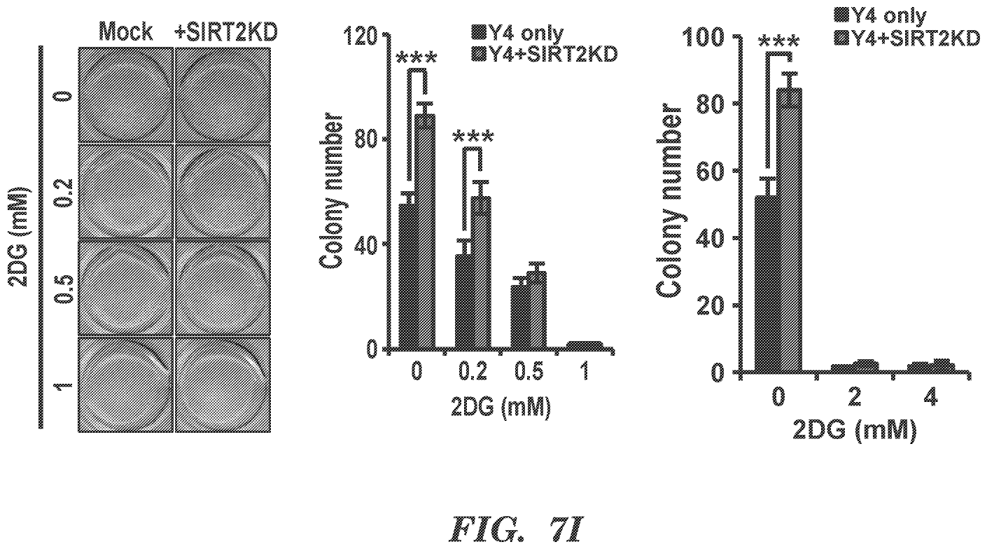

The dynamics of metabolic change by altered SIRT2 expression were also examined during the reprogramming process. As shown in FIG. 7A, 6 days after transfection of Y4, SIRT2 expression was prominently downregulated. Furthermore, decreased OCR and increased ECAR levels were also observed as early as 6 days after transfection, while lactate production was significantly induced at day 9 post-transfection (FIGS. 7B-7D). Importantly, it was found that reprogramming cells with SIRT2 KD resulted in significantly enhanced changes in OCR and ECAR levels and induction of extracellular lactate production compared to control reprogramming cells (FIGS. 7A-7D).

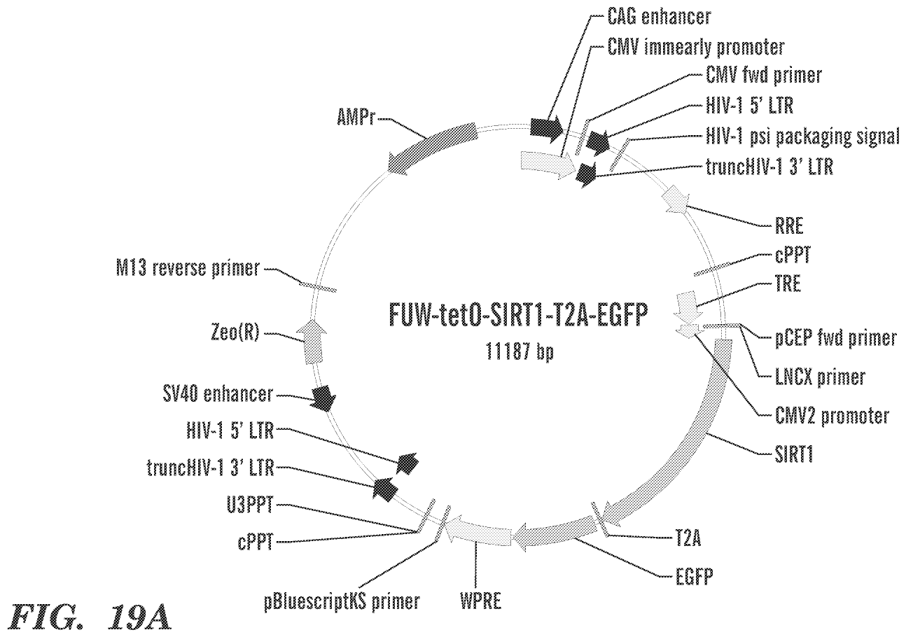

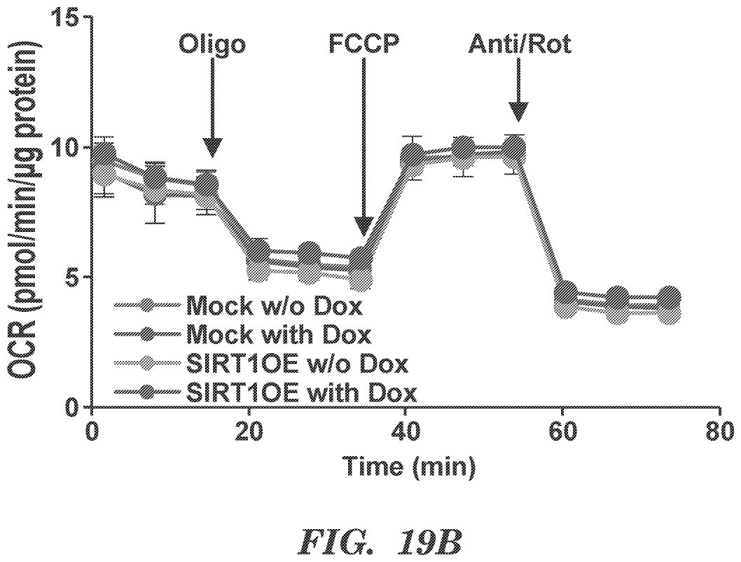

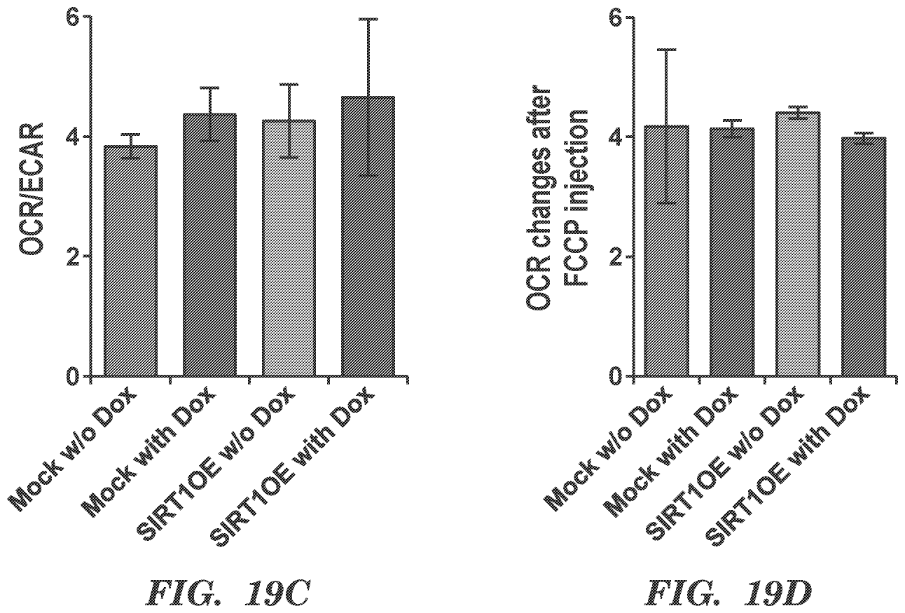

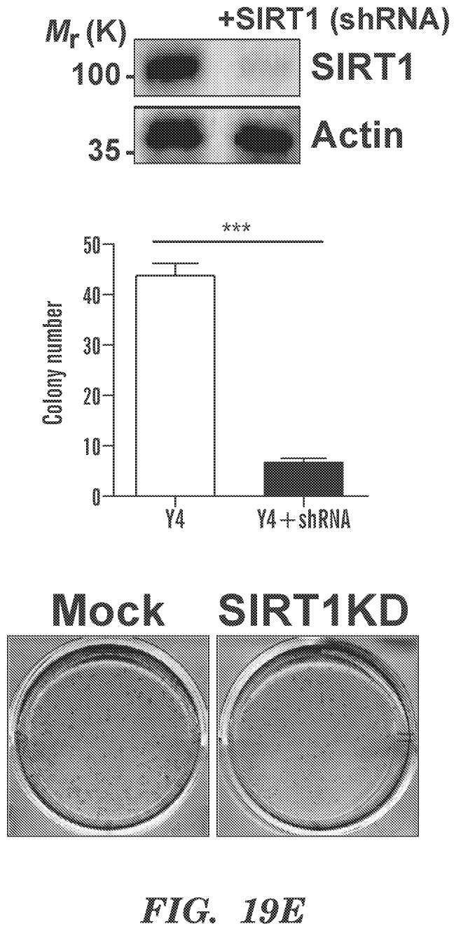

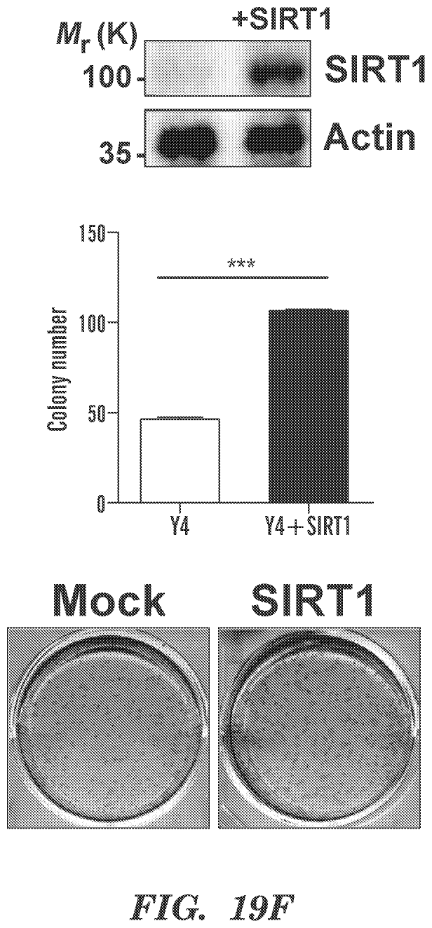

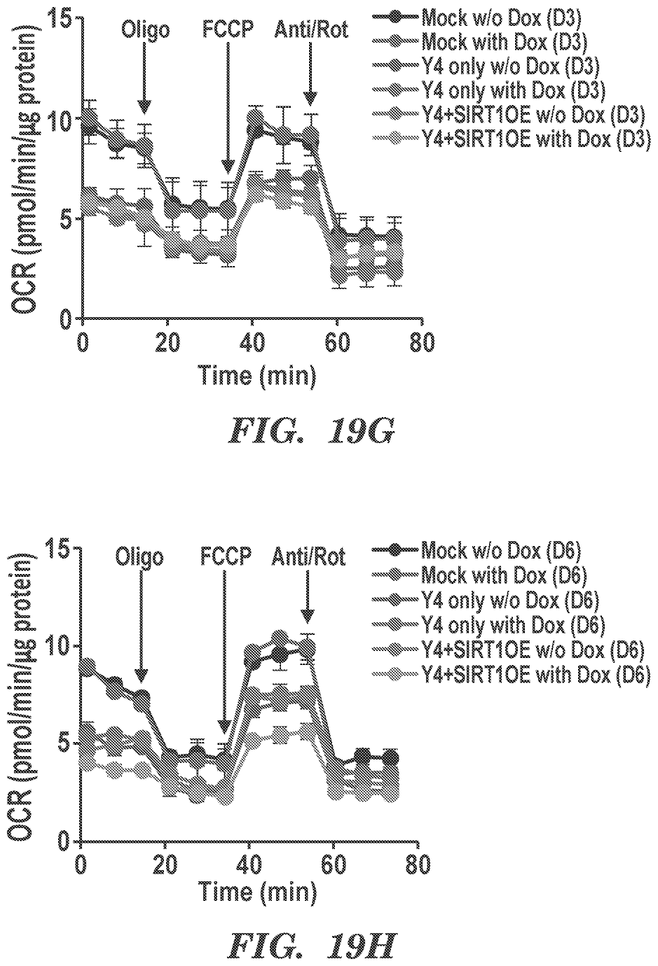

Whether altered SIRT2 expression influences the generation ofiPSCs from fibroblasts was next tested. As shown in FIG. 7E, SIRT2 OE in hDFs interfered with the generation of alkaline phosphatase (AP)-positive iPSC colonies by approximately 80%. In contrast, SIRT2 KD significantly increased the generation of iPSC colonies (FIG. 7F). These results indicate that downregulation of SIRT2 during the reprogramming process is critical for the generation of iPSCs, via enhancing metabolic reprogramming. In addition, it was found that SIRT1 KD prominently reduced the number of iPSC colonies while its overexpression significantly enhanced it (FIGS. 19E and 19F), which is in agreement with previous studies showing a critical role of SIRT1 for induced pluripotency^{32, 39}. However, altered SIRT1 level in hDFs did not influence oxidative metabolism at day 3 (FIGS. 19B-19D). In addition, when SIRT1 was overexpressed in the presence of reprogramming factors, no metabolic change was detected at day 3 during reprogramming (FIG. 19G). Notably, SIRT1 OE appears to enhance metabolic switch at day 6 (FIG. 19H), which is likely due to an indirect effect by enhancing the reprogramming process (FIGS. 19E and 19F). To further test whether enhanced reprogramming by SIRT2 KD depends on elevated glycolysis, the effects of treatment with different concentrations of 2-deoxy-glucose (2DG), a general inhibitor of glycolysis, on metabolic changes and the generation of iPSC colonies were tested. Notably, treatment with 0.2 mM 2DG decreased the glycolytic flux in Y4+SIRT2 KD to the level of Y4 only without 2DG (FIG. 7H), resulting in the generation of iPSC-like colonies to the level of Y4 only without 2DG (FIG. 7I). In addition, when fibroblasts were treated with 0.5 mM 2DG, metabolic changes and increased generation of iPSC-like colonies by SIRT2 KD were abrogated (FIGS. 7G-7I). When fibroblasts were treated with 1 mM or higher concentration of 2DG the generation of iPSC-like colonies was completely blocked. Taken together, these results indicate that enhanced reprogramming by SIRT2 KD is linked to SIRT2's effect on metabolic reprogramming.

miR-200c Suppresses SIRT2 Expression

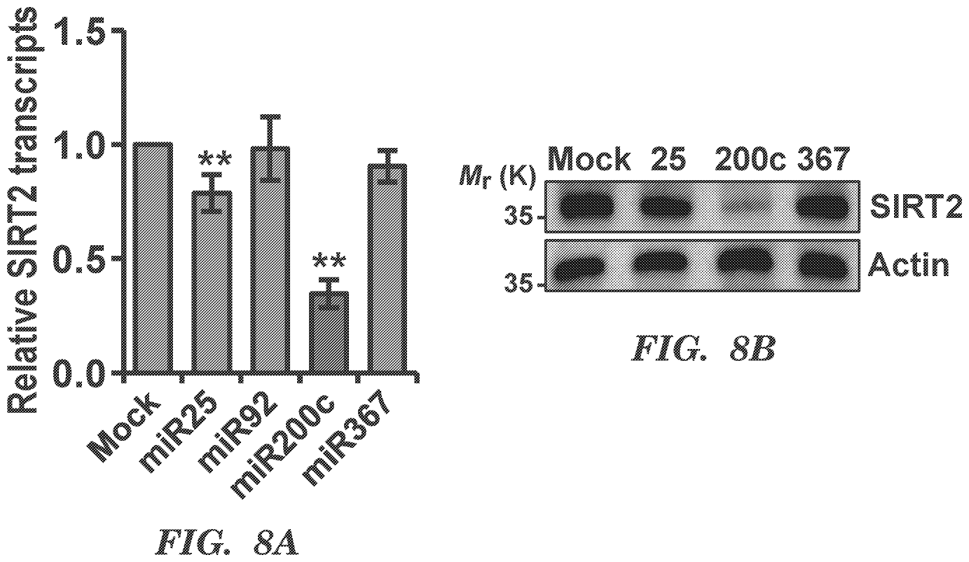

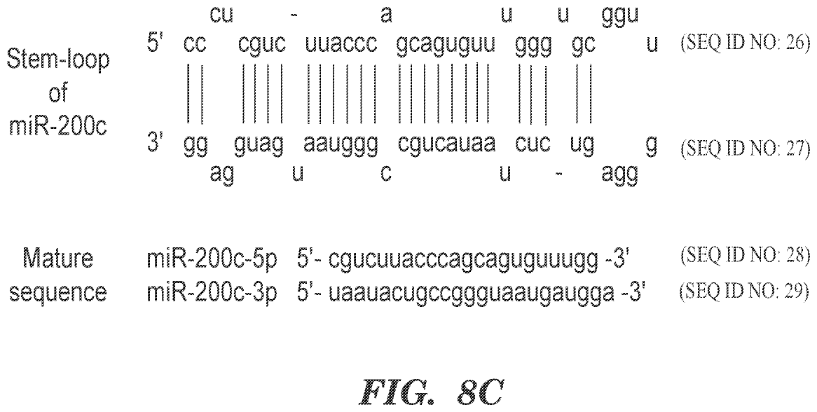

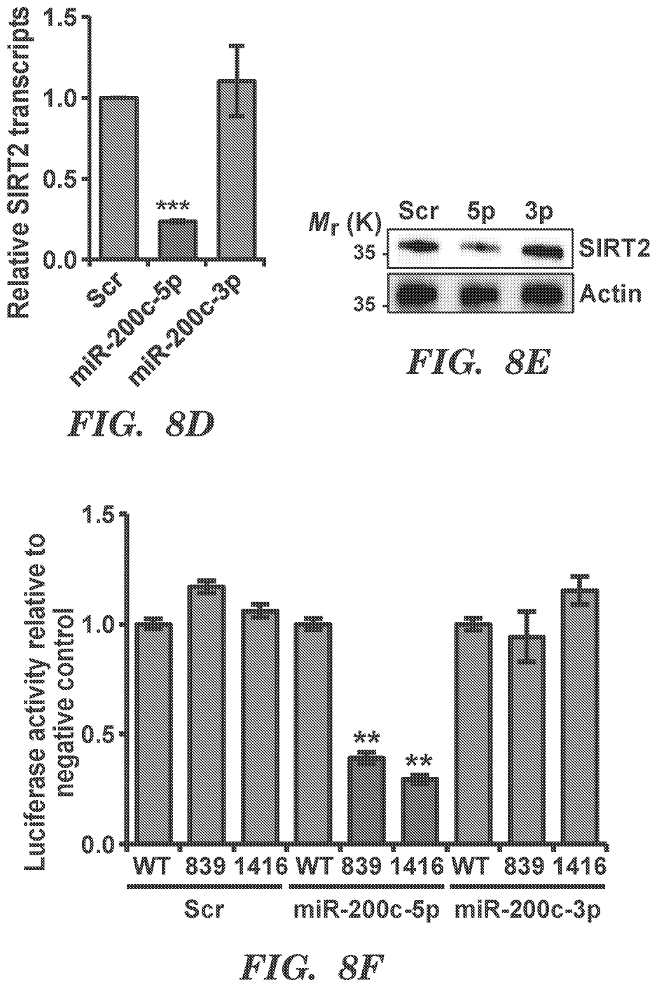

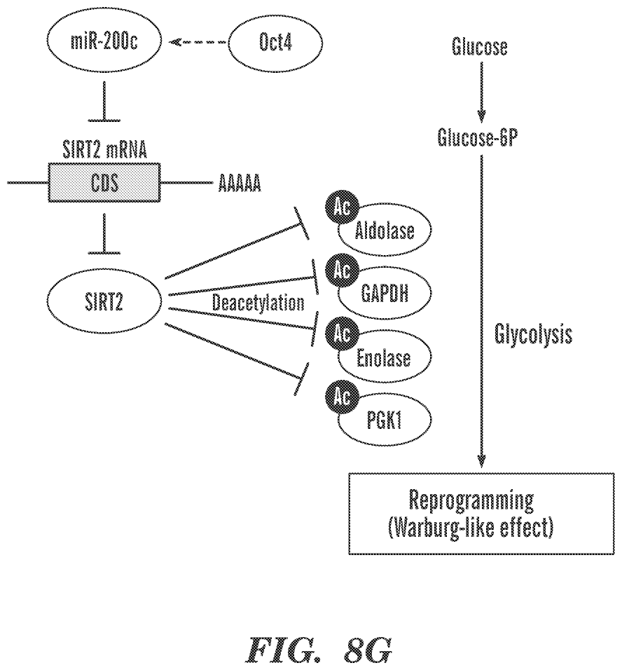

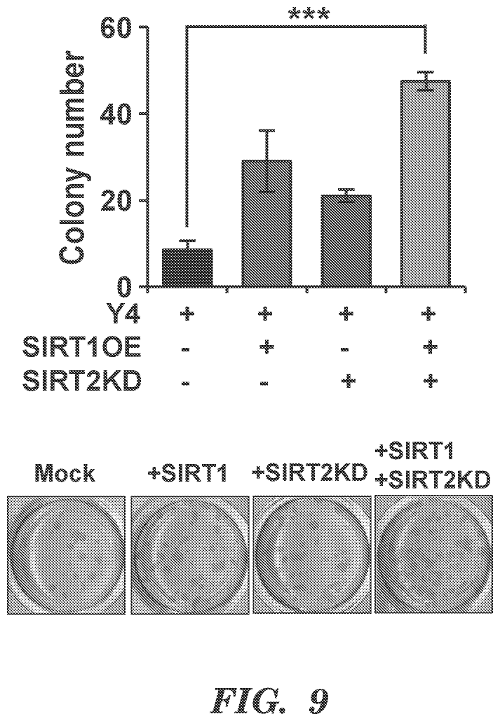

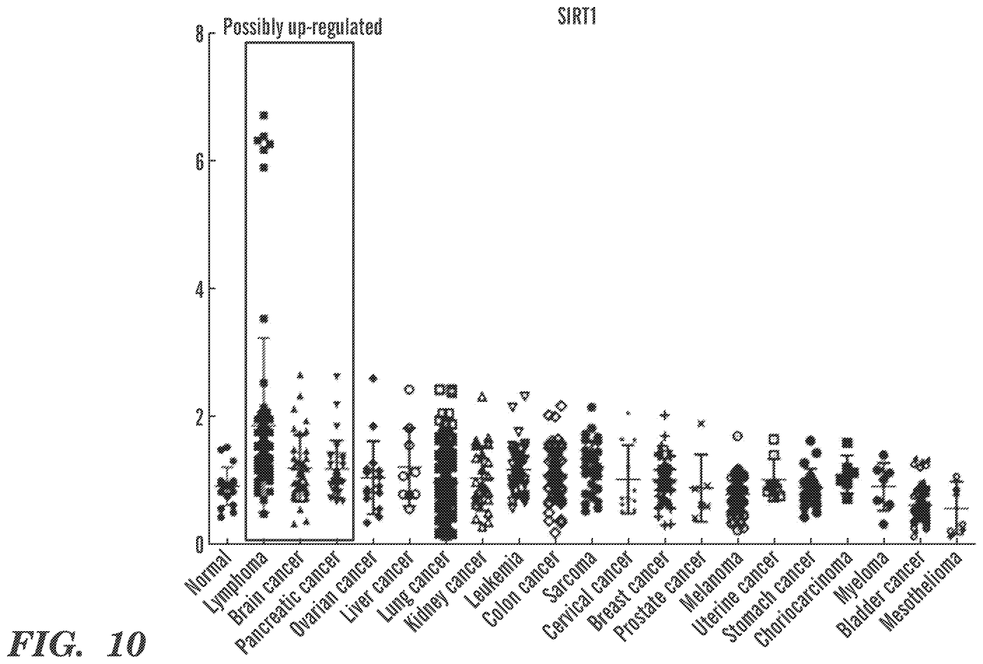

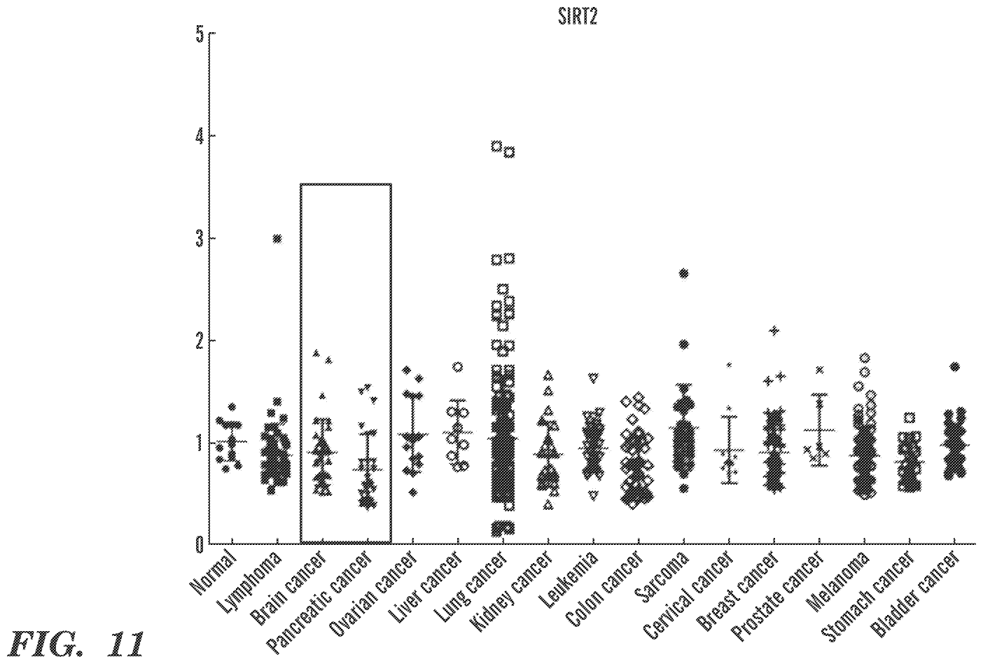

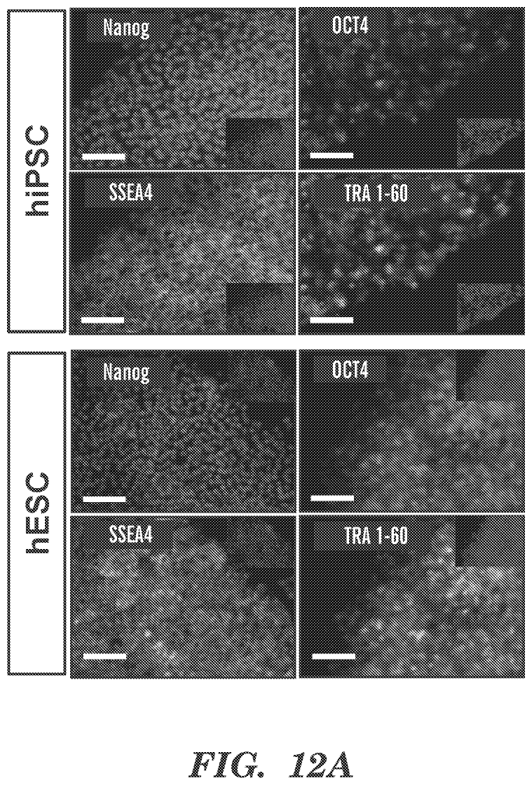

Finally, it was sought to identify the molecular mechanism underlying SIRT2 downregulation during induced pluripotency. In particular, it was speculated that SIRT2 might be regulated by a specific miRNA(s) that are induced by at least one of the reprogramming factors. To address this, miRNA target-prediction analyses using Rna22⁴⁰ was first performed and 656 potential miRNAs that can target the SIRT2 gene were identified. Among these, identified four miRNAs (i.e., miR-25, -92b, -200c, and -367) that belong to the most highly enriched miRNAs in hPSCs⁴¹ were further. Their potential target sites (miRNA-response elements; MREs) in the 5′-untranslated region (UTR) and amino acid coding sequences (CDS) of the SIRT2 gene (Table 7) were also identified. Interestingly, one of these candidates (miR-200c), known to be induced by Oct4⁴², was found to prominently downregulate SIRT2 expression at both the mRNA and protein levels (FIGS. 8A and 8B). Because the prediction analysis used herein showed that SIRT2 could be targeted by miR-200c-5p but not miR-200c-3p (FIG. 8C and Table 7), fibroblasts were transfected with each precursor miRNA (pre-miRNA) oligomer and the effect on the expression levels of the endogenous SIRT2 gene were measured using qRT-PCR and western blot analyses. Transfection of pre-miR-200c-5p significantly decreased the expression level of SIRT2, whereas pre-miR-200c-3p or scrambled oligomers (Scr) did not change SIRT2 mRNA or protein expression (FIGS. 8D and 8E). To validate if miR-200c-5p suppresses SIRT2 expression through the identified MREs, luciferase reporter constructs harboring each of these potential sites were generated. It was found that transfection of pre-miR-200c-5p, but not pre-miR-200c-3p or scrambled sequences, significantly decreased the reporter expression of both MREs (FIG. 8F). These results indicate that Oct4-induced miR-200c-5p downregulates SIRT2 expression by targeting these two MREs residing in the CDS. Taken together, the results presented herein indicate that miR-200c suppresses SIRT2 expression leading to metabolic reprogramming during human induced pluripotency (FIG. 8G).

### DISCUSSION

Here, a molecular signature consisting of SIRT2 downregulation and SIRT1 upregulation in primed hPSCs during the reprogramming process was uncovered, which is critical for induced pluripotency. It was found that SIRT2 KD in human fibroblasts significantly increases the generation of hiPSC colonies while its OE prominently inhibit it. Regulation of SIRT1 expression is also critical for induced pluripotency but in the opposite direction: SIRT1 OE significantly increases the generation of hiPSC colonies while its KD robustly interferes with it. In line with their opposite direction of expression, it appears that SIRT1 and SIRT2 regulate induced pluripotency through distinct mechanisms and targets. For instance, results presented herein highlight that acetylation levels and activities of glycolytic enzymes (e.g., aldolase, PGK1, enolase, and GAPDH) are robustly regulated by SIRT2, but not SIRT1. In agreement with results presented herein, previous studies showed upregulation of SIRT1 in hPSCs ^{31, 32} and SIRT1's important roles for generation of mouse iPSCs ^{32 39}. In addition, the study by Si et al., ³³ showed that SIRT2 is upregulated during in vitro differentiation of mouse ESCs and its KD promotes mesoderm and endoderm lineages while compromising ectoderm differentiation. In contrast, results presented herein show that SIRT2 regulates more fundamental stem cell functions such as metabolism, cell survival/death, and pluripotent differentiation potential in hPSCs. The different functional role(s) of SIRT2 between these two studies possibly reflect species differences (mouse vs. human). Another possibility is that SIRT2 has distinct functional role(s) for different stem cell state. Unlike hESCs and hiPSCs, which represent a primed pluripotent state, mouse ESCs are known to be at a naïve pluripotent state and are energetically bivalent, dynamically switching from glycolysis to OXPHOS on demand⁹.

Recent studies implicate that increased glycolysis is critical for the maintenance or induction of pluripotency^{6, 7, 11-13.} Especially, Moussaieff et al. found that inhibition of glycolysis by BrPA or 2DG causes a rapid loss of pluripotency¹². In contrast, results presented herein showed that SIRT2 OE hPSCs still can be maintained in the undifferentiated state using ESC culture conditions, while they exhibit decreased acetylation levels of glycolytic enzymes and reduced glycolytic metabolism. When hPSCs were exposed to differentiation condition, SIRT2 OE in hPSCs caused further decreased glycolysis, leading to reduced production of lactate, a key metabolite of glycolysis, during early differentiation. It is to be noted that culture conditions (in both ESC maintenance and differentiation) are significantly different between Moussaieff et al. ¹² and findings presented herein. For instance, the chemically defined culture medium (E8TM) containing TGFI3 was used herein, which is known to support undifferentiated proliferation of hPSCs. Indeed, SIRT2 over expression in TGFO-free hPSCs culture condition result in efficient loss of pluripotency and spontaneous differentiation (data not shown). Furthermore, during in vitro differentiation, Moussaieff et al. detected a significant decrease of lactate after 2 days of differentiation while it was only evident after 4 days of differentiation in the experiments presented herein.

Importantly, work presented herein found multiple lines of evidence indicating that SIRT2 is a key regulator of metabolic reprogramming (Warburg-like effect) during human induced pluripotency and critically regulates stem cell fates and functions. Firstly, Dox-induced SIRT2 OE in hESCs robustly altered the acetylation levels and enzymatic activities of glycolytic enzymes, significantly compromising glycolytic metabolism. Secondly, SIRT2 OE in hPSCs caused enhanced OXPHOS and reduced glycolysis, leading to reduction of lactate production. As a result, SIRT2 OE hPSCs exhibit significantly reduced cell proliferation, which may be caused, at least in part, by increased apoptotic cell death via enhanced production of ROS. In addition, SIRT2 OE in hPSCs leads to enhanced pluripotent differentiation potential. Thirdly, SIRT2 KD in human fibroblasts robustly increased acetylation levels and activities of glycolytic enzymes, leading to prominent metabolic switch from OXPHOS to glycolytic metabolism. Fourthly, SIRT2 KD together with the introduction of reprogramming factors into human fibroblasts more rapidly and effectively induced metabolic switch compared to reprogramming factors alone, resulting in more efficient generation of hiPSC colonies. In contrast, altered expression of SIRT1 did not directly influence the metabolic status, further supporting that SIRT1 and SIRT2 regulate the reprogramming process via distinct mechanisms. Taken together, data presented herein indicate that altered levels of SIRT2 during induced pluripotency and differentiation regulate OXPHOS and glycolysis in opposite directions, thus facilitating the metabolic switches. Notably, SIRT2 is the only sirtuin residing primarily in the cytoplasm^{18, 19}, and this may provide a unique advantage to directly control metabolic reprogramming by regulating glycolytic enzymes activities.

The finding that there is a direct correlation between acetylation levels and enzymatic activities is surprising because it was suggested that acetylation is inhibitory to the activities of most enzymes³⁴. For instance, two groups showed that deacetylation of a glycolytic enzyme (phosphoglycerate mutase) by SIRT1 or SIRT2 downregulates its activity^{44, 45}. However, another study reported that the same enzyme could be stimulated through deacetylation by SIRT2⁴⁶ and a recent study showed that GAPDH is activated by acetylation of its K254 residue⁴⁷. Furthermore, increasing GapA acetylation in Salmonella by Pat acetylase treatment increased its glycolysis activity¹⁶. Thus, the functional effect of acetylation appears to be enzyme- and perhaps lysine-specific. To further validate the findings presented herein, LC-MS/MS analyses of Myc-tagged aldolase A (AldoA-Myc) was performed. K111 and K322 were identified as specific SIRT2 target sites and found that K322 critically regulates enzyme activity. K322 resides on an outside surface of AldoA with unknown functional domain, and the new functional data presented herein will provide useful insight into this important enzyme and its regulation in diseases such as cancer.

Interestingly, it was found that SIRT2 is suppressed by miR-200c, a miRNA induced in pluripotent stem cells by Oct4⁴², via binding sites in the sirtuin gene coding sequence. This miRNA enhances metabolic reprogramming via SIRT2 suppression and this appears to be a critical step of induced pluripotency (FIG. 8G). Indeed, enforced SIRT2 OE is highly inhibitory to iPSC reprogramming in human cells. It should be of interest to determine whether this regulation of metabolism by the miR-200c-SIRT2 axis is also important in stem cell function for other types of stem cells (e.g., adult stem cells, naïve pluripotent stem cells, and cancer stem cells). A defect in this process could lead to dysfunctional stem cells and compromised development in embryos or dysfunctional tissues in adults. Further, manipulation of the metabolic control of cell fate and function via the miR-200c-SIRT2 axis may aid translational approaches that use stem cells for regenerative medicine and cell replacement therapy.

Materials and Methods

Cell Culture.

Human dermal fibroblasts (hDFs) were cultured in Dulbecco's modified Minimal Essential Medium (DMEM; Invitrogen, Carlsbad, Calif.) supplemented with 2 mM L-glutamine (Invitrogen), 10% fetal bovine serum (FBS; Invitrogen), 100 U/ml penicillin and 100 μg/ml streptomycin (Invitrogen). For iPSC induction, DMEM/F-12 medium supplemented with 2 mM L-glutamine (Invitrogen), 1 mM p-mercaptoethanol (Invitrogen), 1× non-essential amino acids (NEAA; Invitrogen), 20% knock-out serum replacement (KSR; Invitrogen), 100 U/ml penicillin, 100 μg/ml streptomycin (Invitrogen) and 10 ng/ml basic fibroblast growth factor (bFGF; Invitrogen) was used as the reprogramming medium. Human ESC lines and hiPSC lines were maintained in Essential 8 medium (Invitrogen) using Matrigel® Matrix (Corning Life Sciences, Tewksbury, Mass.) and passaged using 0.5 mM EDTA (Invitrogen) for gentle dissociation.

Plasmid Construction and Lentivirus Production.

Human SIRT1 or SIRT2 was PCR-amplified from hESCs (H9) or hDFs, respectively, then cloned into the pGEM®-T Easy vector (Promega, Madison, Wis.). The 2A sequence of the Thoseaasigna virus (T2A)-linked EGFP was amplified from pCXLE-EGFP plasmid (#27082; Addgene, Cambridge, Mass.) by RT-PCR, cloned into the pGar-T Easy vector. The SIRT1 and SIRT2 fragments were then cut off from the corresponding vectors and inserted into the pGEM-T-T2A-EGFP to generate pGEM-T-SIRT1-T2A-EGFP and pGEM-T-SIRT2-T2A-EGFP, respectively. The SIRT1-T2A-EGFP and SIRT2-T2A-EGFP constructs were confirmed by sequencing and then introduced into the EcoRI site of FUW-tetO vector (Addgene), respectively. Human AldoA-Myc constructs, the AldoA fragment was PCR-amplified from hESCs (H9), and then cloned into the pcDNA3.1-Myc/His vector (Invitrogen). For the psicheck2 constructs, the CDS fragments were cloned in downstream of a Renilla luciferase open reading frame. Point mutations of AldoA were generated by site-directed mutagenesis using a QuickChange II XL Site-Directed Mutagenesis kit (Agilent Technologies, Santa Clara, Calif.). The primers are listed in Table 6. FUW-tetO-based lentiviral vectors containing the other individual reprogramming factors for Oct4 (#20726), Sox2 (#20724), Klf4 (#20725) or c-Myc (#20723) were purchased from Addgene. The polycistronic human STEMCCA lentiviral vector⁴⁸ was kindly provided by Dr. Gustavo Mostoslaysky (Boston University). Genetic knockdown of SIRT1 or SIRT2 was carried out using lentiviral shRNA plasmids targeting human SIRT1 (RHS3979-201750186, RHS3979-201750188, RHS3979-201750189, and RHS3979-201750190) or human SIRT2 (RHS3979-201797165, RHS3979-201768981, RHS3979-201768982, RHS3979-201768983, RHS3979-201768984, and RHS3979-201768985) that were obtained from GE Healthcare Dharmacon (Lafayette, Colo.).

For lentivirus production, lentiviral vectors were co-transfected by packaging plasmids into 293T cells which were maintained in DMEM supplemented with 10% FBS using Lipofectamine 2000 (Invitrogen) according to the manufacturer's instruction. The viral supernatant was harvested at 48 hours (h) after transfection and filtered using 0.45 pm Millex-HV (Millipore) filters to remove cell debris.

Human iPSC Induction.

Human iPSCs were generated using lentiviral particles by inducible lentiviral vectors or STEMCCA vectors to introduce the OSKM factors (Oct4, Sox2, Klf4, and c-Myc) into fibroblasts⁴⁹. ES-like colonies formed after 3 weeks of viral infection and the observed ES-like colonies were handpicked and transferred onto mouse feeder cells (MEF)-plated or Matrigel-coated tissue culture plates to generate iPSC lines. iPSC colonies were mechanically picked until iPSC lines were established.

Live Cell Metabolic Analysis.

Oxygen consumption rate (OCR) and extracellular acidification rates (ECAR) were measured using the XFp8 or XF24 analyzer (Seahorse Bioscience, MA) according to the manufacturer's instruction. Briefly, cells were plated into wells of an XF cell culture microplate and incubated at 37° C. in a CO₂ incubator for 24 h to ensure attachment. The assay was started after cells were equilibrated for 1 h in XF assay medium supplemented with 10 mM glucose, 5 mM sodium pyruvate and 2 mM glutamine in a non-CO₂ incubator. Mitochondrial activity between hDFs and hESCs/parental hDFs and iPSCs were monitored through sequential injections of 1 μM oligomycin, 0.3 pLM FCCP and 1 μM rotenone/antimycin A to calculate basal respiration rates (baseline OCR—rotenone/antimycin A OCR), ATP dependent (basal respiration rate—oligomycin OCR), maximum respiration (FCCP OCR— rotenone/antimycin A OCR), and oxidative reserve (maximum respiration rate—basal respiration rate). Glycolytic processes were measured by serial injections of 10 mM glucose, 1 μM oligomycin, and 100 mM 2-deoxyglucose to calculate basal glycolytic rate, glycolytic capacity (in response to oligomycin), and glycolytic reserve (glycolytic capacity—basal rate). Each plotted value was normalized to total protein quantified using a Bradford protein assay (Bio-Rad).

Immunoprecipitation.

For immunoprecipitation assays, hESCs and hDFs lysates were incubated with specific antibodies against acetyl-Lys, aldolase, enolase, PGK1 or GAPDH at 4° C. overnight. After addition of protein A/G UltraLink resin, samples were incubated at 4° C. for 2 h. Beads were washed three times with PBS and proteins were released from the beads by boiling in SDS-sample loading buffer and analyzed by SDS-PAGE.

Liquid Chromatography Mass Spectrometry (LC-MS/MS).

For identification of acetylated proteins, hESCs or hDFs (control) were plated in 100 mm dishes, grown in STEMPRO® hESC SFM up to 60-70% confluence. Cells were collected, washed with PBS and lysed (50 mM Tris-HCl, pH 7.4, 150 mM NaCl, 1 mM EDTA, 1 mM EGTA, 0.5% NP-40, 1% SDS, and protease inhibitor cocktail). Whole cell lysate from hESCs and hDFs were incubated for 10 min on ice followed by centrifugation at 14,000×g for 15 min at 4° C. Supernatants were collected and pellets were discarded. Protein concentrations were determined using the BCA assay (Pierce, Rockford, Ill.) using bovine serum albumin (BSA) as standard. For immunoprecipitation assays, 500 μg of hESC and hDFs lysates were incubated with anti-acetyl-Lys antibody at 4° C. for overnight. After addition of Protein A/G UltraLink resin samples were incubated at 4° C. for 2 h. Beads were washed three times with PBS and proteins were released from the beads by addition of SDS-sample loading buffer. The eluted proteins were analyzed by SDS-PAGE and the gel stained with Coomassie Blue. For LC-MS/MS analyses, the gel was de-stained and bands cut and processed as follows. Briefly, acetylated proteins bands were divided into 10 mm sections and subjected to in-gel digestion with trypsin. The tryptic digests were separated by on-line reversed-phase chromatography using a Thermo Scientific Eazy nano LC II UHPLC equipped with an autosampler using a reversed-phase peptide trap EASY-Column (100 μm inner diameter, 2 cm length) and a reversed-phase analytical EASY-Column (75 gm inner diameter, 10 cm length, 3 pm particle size), both from Thermo Scientific, followed by electrospray ionization using a 30 gm (i.d.) nanobore stainless steel online emitter (Thermo Scientific) and a voltage set at 2.6 V., at a flow rate of 300 nl/min. The chromatography system was coupled on-line with an LTQ mass spectrometer. Spectra were searched against the Human IPI v3.7 DB using the Sorcerer 2 IDA Sequest-based search algorithm, and comparative analysis of proteins identified in this study was performed using Scaffold 4. LC-MS/MS analysis was performed at the Biopolymers & Proteomics Core Facility of the David H. Koch Institute at MIT and at the Medicinal Bioconvergence Research Center at Seoul National University. To compare protein acetylation between hESCs and hDFs, the acetylated proteins in both samples were quantified based on spectral counts. The spectral counts were first normalized to ensure that average spectral counts per protein was the same in the two data sets⁵⁰. A G test was used to judge statistical significance of protein abundance differences ⁵¹. Briefly, the G value of each protein was calculated as follows:

G=2(Si×ln[Si/((Si+S2)/2)]+S2/ln[S2/(S1±S2)21));

wherein S₁ and S₂ are the detected spectral counts of a given protein in any of two samples for comparison. Although the theoretical distribution of the G values is complex, these values approximately fit to the ₇₂ distribution (1 degree of freedom), allowing the calculation of related p values⁵¹. For identification of acetylation sites on AldoA, Myc-conjugated AldoA proteins were pulled down by immunoprecipitation via Myc antibody from 293T cells infected with AldoA-Myc-overexpressing plasmid together with empty or SIRT2 KD plasmid. The AldoA-Myc band was excised, digested with chymotrypsin, and analyzed by LTQ-Orbitrap ion-trap mass spectrometer from Thermo Scientific (Taplin Mass Spectrometry Facility, Harvard University, Boston, Mass.; found on the world wide web at https://taplin.med.harvard.edu/home).

Western Blot Analysis.

Samples (50 μg) were loaded onto a 12% SDS-PAGE and separated by electrophoresis followed by transfer onto a piece of Immun-Blot PVDF membrane (Bio-Rad, Hercules, Calif.). After transfer, the membrane was blocked at room temperature with Tris-buffered saline (TBS) containing 0.1% Tween-20 and 5% (w/v) skim milk for 3-5 h and then incubated overnight at 4° C. with primary antibody. The membrane was washed three times with TBS containing 0.05% Tween-20 (TBST) and then incubated for 2 h with the appropriate secondary antibody (Pierce, Rockford, Ill.). After washing twice with TBST and once with TBS, bound antibodies were detected by chemiluminescence using the SuperSignal® West Pico kit (Pierce). Antibodies against acetyl-Lys (#9441; 1:1000) and Enolase (#3810; 1:1000) were purchased from Cell Signaling Technology (Danvers, Mass.), actin (ab8227; 1:1000), tubulin (ab4074; 1:1000), acetylated-tubulin (ab24610; 1:1000), total OXPHOS cocktail (ab110413; 1:250), SIRT1 (ab32441; 1:1000), and SIRT2 (ab51023; 1:1000) from Abcam (Cambridge, Mass.), Aldolase A (sc-12059; 1:1000), PGK1 (sc-130335; 1:1000), GAPDH (sc-32233; 1:1000) from Santa Cruz Biotechnologies (Santa Cruz, Calif.). horseradish peroxidase-conjugated Veriblot for 1P secondary antibody (ab131366; Abcam) were used to facilitate detection of immunoprecipitated proteins without co-detecting the IgG heavy and light chains. The PVDF membrane was stripped by washing three times with TBST followed by incubation at 50° C. for 30 min with shaking in stripping buffer (62.5 mM Tris-HC 1, pH 6.7, 100 mM13-mercaptoethanol, and 2% SDS). After incubation, the membrane was washed several times with TBST. Stripped membranes were blocked and probed with primary and secondary antibodies as previously described.

Immunofluorescence.

For immunofluorescence assay, cells were immediately fixed (2% formaldehyde, 100 mM KCl, 200 mM sucrose, 1 mM EGTA, 1 mM MgCl2, 10 mM PIPES, pH 6.8) for 10 min, washed with PBS and then treated with permeabilization buffer (0.2% Triton X-100, 100 mM KCl, 200 mM sucrose, 1 mM EGTA, 1 mM MgCl₂, 10 mM PIPES, pH 6.8) for 10 min. Cells were washed with PBS three times and incubated with blocking solution containing 3% BSA in PBS for 15 min. Cells were washed with PBS three times and incubated with primary antibodies in blocking solution at 4° C. overnight. Oct4 (sc-5279; 1:500) and Nanog (sc-33759; 1:500) antibodies were obtained from Santa Cruz Biotechnologies, SSEA4 (MAB4304; 1:500) and TRA-1-60 (MAB4360; 1:500) antibodies from EMD Millipore (Billerica, Mass.), Otx2 (AF1979; 1:500), Sox17 (AF1924; 1:500) and Brachyury (AF2085; 1:500) antibodies from R&D Systems, Inc. (Minneapolis, Minn.). Cells were washed with PBS three times and incubated with Alexa Fluor conjugated secondary antibodies (Alexa Fluor® 488 goat anti-mouse (A11001; Invitrogen) and Alexa Fluor® 568 donkey anti-rabbit (A10042; Invitrogen)) in blocking solution. After washing with PBS, nuclei were stained with Hoechst33342 (H3570; Invitrogen). Each image was examined using a confocal laser-scanning microscope (Olympus America Inc., Melville, N.Y.).

Quantitative Reverse Transcription Polymerase Chain Reaction (qRT-PCR).

Total RNA was extracted from cells by using the Direct-zol RNA purification Kit (Zymo research, Irvine, Calif.) and cDNA was synthesized using the ThermoScript™ RT-PCR system (Invitrogen). For quantitative analysis, qRT-PCR (Bio-Rad) was performed using SsoAdvanced SYBR Green supermix (Bio-Rad) with target genes specific primers. The expression level of each gene was shown as relative value following normalization against that of the 13-actin gene. Primers used in this study are listed in Table 8.

ATP Determination Assay.

Cellular ATP concentration was measured by using an ATP determination kit (Molecular Probe, Carlsbad, Calif.). Cells (iPSCs and parental hDFs/hESCs and hDFs) were washed three times with PBS and lysed by addition of water and boiled for 5 min. Cell lysates were collected by centrifugation for 15 min at 4° C. ATP chemiluminescent detection was performed using firefly luciferase and luciferin and measured by a SpectraMax L (Molecular Devices, Sunnyvale, Calif.). Cell lysates protein concentrations were determined using the BCA assay (Bio-Rad) and RLU (relative luminescent unit) were normalized according to protein concentrations.

Neuronal and Spontaneous Differentiation.

Neuronal differentiation was performed as described previously with slight modifications⁵². Briefly, hESCs were dissociated and embryoid bodies (EB) were allowed to form for 1 week after plating on bacterial dishes in hESC medium without bFGF. EBs were allowed to attach to tissue culture dish and neuronal precursors were selected by incubation in serum-free ITSFn (Insulin-Transferrin-Selenium-Fibronectin) medium for 30 days. hESCs and hiPSCs in vitro spontaneous differentiation was performed by culturing in serum-free ITSFn medium for different periods up to 12 days without EB formation.

Fluorescence-Based Competition Assay.

Fluorescence-based competition assay was performed as described previously with slight modifications^{37, 38}. Briefly, GFP expressing hESCs (GFP) or SIRT2 (and GFP)-inducible hESCs (SIRT2) were mixed with wild type hESCs (GFP⁻) and cultured in matrigel-coated 6 well plates. Every 5 days (one passage) cells were dissociated using accutase (A6964; Sigma-Aldrich, St. Louis, Mo.) and replated. At each passage, the proportion of GFP⁺/GFP⁻ cells was measured by flow cytometry on a BD Accuri flow cytometer using the Accuri C6 data analysis software (Ann Arbor, Mich.). Analyses were carried out for six consecutive passages.

Enzyme Activity Assay.

Enzyme activity of aldolase (#K665-100), enolase (#K691-100), and GAPDH (#K640-100) was measured using an enzymatic colorimetric assay kit (Biovision, Milpitas, Calif.) according to the manufacturer's instruction. All samples were assayed in triplicate wells, and data are presented as mean±SEM.

Proliferation Assay.

Cells were detached using accutase for 10 min and suspended in ESC medium and counted using a hemocytometer. An equal number of cells (1×10⁴ cells/well) were seeded on matrigel-coated 12 well plates. The total number of cells per well was determined at 2, 4, 6 days post-seeding using a hemocytometer.

Annexin Stainin.

For apoptosis analysis, cells were washed twice with cold PBS, and then stained with annexin V-PE and 7-AAD (559763; BD Biosciences), and analyzed by flow cytometer.

Luciferase Reporter Assay.

The Promega dual luciferase assay kit was used to perform the luciferase assay according to the manufacturer's instruction. In brief, cell lysates were analyzed for luciferase activity using the dual luciferase system in which two luciferase enzymes, one (from Renilla reniformis) containing the experimental target sequence and another (from firefly) containing the control. The Renilla/firefly luciferase ratios were normalized against the empty psicheck-2 vector and averaged over 6 replicates.

Cellular ROS Measurements.

Intracellular ROS levels were determined using a CeliROX® Deep Red Oxidative Stress Reagent (C10422; Life technologies) according to the manufacturer's instruction.

Lactate Assay.

Extracellular lactate production was measured using L-Lactate assay kit (700510; Cayman Chemical, Ann Arbor, Mich.) according to the manufacturer's instruction.

Statistical Analysis.

The graphical data are presented as mean±SEM. For multiple group comparisons one-way analysis of variance (ANOVA) was used followed by Bonferroni post-test analysis. For two groups comparisons Student's t test was used. Statistically significant differences are indicated as follows: *p<0.05; ″p<0.01; ***p<0.005; ****p<0.001.

```
Nucleic acid sequence encoding SIRT1 (SEQ ID NO: 2)
(SEQ ID NO: 2)
                          atgtttga tattgaatat ttcagaaaag atccaagacc attcttcaag
tttgcaaagg aaatatatcc tggacaattc cagccatctc tctgtcacaa attcatagcc
ttgtcagata aggaaggaaa actacttcgc aactataccc agaacataga cacgctggaa
caggttgcgg gaatccaaag gataattcag tgtcatggtt cctttgcaac agcatcttgc
ctgatttgta aatacaaagt tgactgtgaa gctgtacgag gagatatttt taatcaggta
gttcctcgat gtcctaggtg cccagctgat gaaccgcttg ctatcatgaa accagagatt
gtgttttttg gtgaaaattt accagaacag tttcatagag ccatgaagta tgacaaagat
gaagttgacc tcctcattgt tattgggtct tccctcaaag taagaccagt agcactaatt
ccaagttcca taccccatga agtgcctcag atattaatta atagagaacc tttgcctcat
ctgcattttg atgtagagct tcttggagac tgtgatgtca taattaatga attgtgtcat
aggttaggtg gtgaatatgc caaactttgc tgtaaccctg taaagctttc agaaattact
gaaaaacctc cacgaacaca aaaagaattg gcttatttgt cagagttgcc acccacacct
cttcatgttt cagaagactc aagttcacca gaaagaactt caccaccaga t
Nucleic acid sequence encoding SIRT2 (SEQ ID NO: 3)
(SEQ ID NO: 3)
                                                                                        gcagacatgg acttcctgcg
gaacttattc tcccagacgc tcagcctggg cagccagaag gagcgtctgc tggacgagct
gaccttggaa ggggtggccc ggtacatgca gagcgaacgc tgtcgcagag tcatctgttt
ggtgggagct ggaatctcca catccgcagg catccccgac tttcgctctc catccaccgg
cctctatgac aacctagaga agtaccatct tccctaccca gaggccatct ttgagatcag
ctatttcaag aaacatccgg aacccttctt cgccctcgcc aaggaactct atcctgggca
gttcaagcca accatctgtc actacttcat gcgcctgctg aaggacaagg ggctactcct
gcgctgctac acgcagaaca tagataccct ggagcgaata gccgggctgg aacaggagga
cttggtggag gcgcacggca ccttctacac atcacactgc gtcagcgcca gctgccggca
cgaatacccg ctaagctgga tgaaagagaa gatcttctct gaggtgacgc ccaagtgtga
agactgtcag agcctggtga agcctgatat cgtctttttt ggtgagagcc tcccagcgcg
tttcttctcc tgtatgcagt cagacttcct gaaggtggac ctcctcctgg tcatgggtac
ctccttgcag gtgcagccct ttgcctccct catcagcaag gcacccctct ccacccctcg
cctgctcatc aacaaggaga aagctggcca gtcggaccct ttcctgggga tgattatggg
cctcggagga ggcatggact ttgactccaa gaaggcctac agggacgtgg cctggctggg
tgaatgcgac cagggctgcc tggcccttgc tgagctcctt ggatggaaga aggagctgga
ggaccttgtc cggagggagc acgccagcat agatgcccag tcgggggcgg gggtccccaa
ccccagcact tcagcttccc ccaagaagtc cccgccacct gccaaggacg aggccaggac
aacagagagg gagaaacccc agtgacagct
Nucleic acid sequence encodig pCXLE-miR-302s/200c (SEQ ID NO: 199)
(SEQ ID NO: 199)
tcgacattgattattgactagttattaatagtaatcaattacggggtcattagttcatagcccatat
atggagttccgcgttacataacttacggtaaatggcccgcctggctgaccgcccaacgacccccgcc
cattgacgtcaataatgacgtatgttcccatagtaacgccaatagggactttccattgacgtcaatg
ggtggagtatttacggtaaactgcccacttggcagtacatcaagtgtatcatatgccaagtacgccc
cctattgacgtcaatgacggtaaatggcccgcctggcattatgcccagtacatgaccttatgggact
ttcctacttggcagtacatctacgtattagtcatcgctattaccatggtcgaggtgagccccacgtt
ctgcttcactctccccatctcccccccctccccacccccaattttgtatttatttattttttaatta
ttttgtgcagcgatgggggcggggggggggggggggcgcgcgccaggcggggcggggcggggcgagg
ggcggggcgggcgaggcggagaggtgcggcggcagccaatcagagcggcgcgctccgaaagtttcct
tttatggcgaggcggcggcggcggcggccctataaaaagcgaagcgcgcggcgggcgggagtcgctg
cgcgctgccttcgccccgtgccccgctccgccgccgcctcgcgccgcccgccccggctctgactgac
cgcgttactcccacaggtgagcgggcgggacggcccttctcctccgggctgtaattagcgcttggtt
taatgacggcttgtttcttttctgtggctgcgtgaaagccttgaggggctccgggagggccctttgt
gcggggggagcggctcggggggtgcgtgcgtgtgtgtgtgcgtggggagcgccgcgtgcggctccgc
gctgcccggcggctgtgagcgctgcgggcgcggcgcggggctttgtgcgctccgcagtgtgcgcgag
gggagcgcggccgggggcggtgccccgcggtgcggggggggctgcgaggggaacaaaggctgcgtgc
ggggtgtgtgcgtgggggggtgagcagggggtgtgggcgcgtcggtcgggctgcaaccccccctgca
cccccctccccgagttgctgagcacggcccggcttcgggtgcggggctccgtacggggcgtggcgcg
gggctcgccgtgccgggcggggggtggcggcaggtgggggtgccgggcggggcggggccgcctcggg
ccggggagggctcgggggaggggcgcggcggcccccggagcgccggcggctgtcgaggcgcggcgag
ccgcagccattgccttttatggtaatcgtgcgagagggcgcagggacttcctttgtcccaaatctgt
gcggagccgaaatctgggaggcgccgccgcaccccctctagcgggcgcggggcgaagcggtgcggcg
ccggcaggaaggaaatgggcggggagggccttcgtgcgtcgccgcgccgccgtccccttctccctct
ccagcctcggggctgtccgcggggggacggctgccttcgggggggacggggcagggcggggttcggc
ttctggcgtgtgaccggcggctctagagcctctgctaaccatgttcatgccttcttctttttcctac
agctcctgggcaacgtgctggttattgtgctgtctcatcattttggcaaagaattc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]














tctagaagg
gctcaccaggaagtgtccccagggactcgggtggtggggggatgggagccagggatctgcagctttt
ccgcagggatcctgggcctgaagctgcctgacccaaggtgggcgggctgggcgggggccctcgtctt
acccagcagtgtttgggtgcggttgggagtctctaatactgccgggtaatgatggaggcccctgtcc
ctgtgtcagcaacatccatcgcctcaggtccccagcccttagctggctgcagccccctccccacttc
ccacgcaccccggaagcccctcgtcttgagctgagagcgttgcacaaggggtggttcttgttggctg
gctgccactaagggacacaatgggccccagcccctcctcccacccagtgcgatttgtcacctggtgg
atccagaacccacagtcgaccttgagcttggggttggctcgccccctctcaagagacctcacctggc
ctgtggccagggtcccctgtagcaactggtgagcgcgcaccgtagttctctgtcggccggccctggg
tccatcttccagtacagtgttggatggtctaattgtgaagctcctaacactgtctggtaaagatggc
tcccgggtgggttctctcggcagtaaccttcagggagccctgaagaccatggaggactactgaccaa
caacctctgaccttcacccctctggatgggggacgaatcactaggcaaaggggaacaatgggaagga
gacagaattcaagcttcggggactagtcatatgataatcaacctctggattacaaaatttgtgaaag
attgactggtattcttaactatgttgctccttttacgctatgtggatacgctgctttaatgcctttg
tatcatgctattgcttcccgtatggctttcattttctcctccttgtataaatcctggttgctgtctc
tttatgaggagttgtggcccgttgtcaggcaacgtggcgtggtgtgcactgtgtttgctgacgcaac
ccccactggttggggcattgccaccacctgtcagctcctttccgggactttcgctttccccctccct
attgccacggcggaactcatcgccgcctgccttgcccgctgctggacaggggctcggctgttgggca
ctgacaattccgtggtgttgtcggggaagctgacgtcctttccatggctgctcgcctgtgttgccac
ctggattctgcgcgggacgtccttctgctacgtcccttcggccctcaatccagcggaccttccttcc
cgcggcctgctgccggctctgcggcctcttccgcgtcttcgccttcgccctcagacgagtcggatct
ccctttgggccgcctccccgcatcggtaaattcactcctcaggtgcaggctgcctatcagaaggtgg
tggctggtgtggccaatgccctggctcacaaataccactgagatctttttccctctgccaaaaatta
tggggacatcatgaagccccttgagcatctgacttctggctaataaaggaaatttattttcattgca
atagtgtgttggaattttttgtgtctctcactcggaaggacatatgggagggcaaatcatttaaaac
atcagaatgagtatttggtttagagtttggcaacatatgcccatatgctggctgccatgaacaaagg
ttggctataaagaggtcatcagtatatgaaacagccccctgctgtccattccttattccatagaaaa
gccttgacttgaggttagattttttttatattttgttttgtgttatttttttctttaacatccctaa
aattttccttacatgttttactagccagatttttcctcctctcctgactactcccagtcatagctgt
ccctcttctcttatggagatccctcgacctgcagcccaagcttggcgtaatcatggtcatagctgtt
tcctgtgtgaaattgttatccgctcacaattccacacaacatacgagccggaagcataaagtgtaaa
gcctggggtgcctaatgagtgagctaactcacattaattgcgttgcgctcactgcccgctttccagt
cgggaaacctgtcgtgccagcggatctcaattccgatcatattcaataacccttaatataacttcgt
ataatgtatgctatacgaagttattaggtctgaagaggagtttacgtccagccaagcttaggatcaa
ttctcatgtttgacagcttatcatcgataagctgatcctcacaggccgcacccagcttttcttccgt
tgccccagtagcatctctgtctggtgaccttgaagaggaagaggaggggtcccgagaatccccatcc
ctaccgtccagcaaaaagggggacgaggaatttgaggcctggcttgaggctcaggacgcaaatcttg
aggatgttcagcgggagttttccgggctgcgagtaattggtgatgaggacgaggatggttcggagga
tggggaattttcagacctggatctgtctgacagcgaccatgaaggggatgagggtgggggggctgtt
ggagggggcaggagtctgcactccctgtattcactgagcgtcgtctaataaagatgtctattgatct
cttttagtgtgaatcatgtctgacgaggggccaggtacaggacctggaaatggcctaggagagaagg
gagacacatctggaccagaaggctccggcggcagtggacctcaaagaagagggggtgataaccatgg
acgaggacggggaagaggacgaggacgaggaggcggaagaccaggagccccgggcggctcaggatca
gggccaagacatagagatggtgtccggagaccccaaaaacgtccaagttgcattggctgcaaaggga
cccacggtggaacaggagcaggagcaggagcgggaggggcaggagcaggaggggcaggagcaggagg
aggggcaggagcaggaggaggggcaggaggggcaggaggggcaggaggggcaggagcaggaggaggg
gcaggagcaggaggaggggcaggaggggcaggaggggcaggagcaggaggaggggcaggagcaggag
gaggggcaggaggggcaggagcaggaggaggggcaggaggggcaggaggggcaggagcaggaggagg
ggcaggagcaggaggaggggcaggaggggcaggagcaggaggaggggcaggaggggcaggaggggca
ggagcaggaggaggggcaggagcaggaggggcaggaggggcaggaggggcaggagcaggaggggcag
gagcaggaggaggggcaggaggggcaggaggggcaggagcaggaggggcaggagcaggaggggcagg
agcaggaggggcaggagcaggaggggcaggaggggcaggagcaggaggggcaggaggggcaggagca
ggaggggcaggaggggcaggagcaggaggaggggcaggaggggcaggagcaggaggaggggcaggag
gggcaggagcaggaggggcaggaggggcaggagcaggaggggcaggaggggcaggagcaggaggggc
aggaggggcaggagcaggaggaggggcaggagcaggaggggcaggagcaggaggtggaggccggggt
cgaggaggcagtggaggccggggtcgaggaggtagtggaggccggggtcgaggaggtagtggaggcc
gccggggtagaggacgtgaaagagccagggggggaagtcgtgaaagagccagggggagaggtcgtgg
acgtggagaaaagaggcccaggagtcccagtagtcagtcatcatcatccgggtctccaccgcgcagg
ccccctccaggtagaaggccatttttccaccctgtaggggaagccgattattttgaataccaccaag
aaggtggcccagatggtgagcctgacgtgcccccgggagcgatagagcagggccccgcagatgaccc
aggagaaggcccaagcactggaccccggggtcagggtgatggaggcaggcgcaaaaaaggagggtgg
tttggaaagcatcgtggtcaaggaggttccaacccgaaatttgagaacattgcagaaggtttaagag
ctctcctggctaggagtcacgtagaaaggactaccgacgaaggaacttgggtcgccggtgtgttcgt
atatggaggtagtaagacctccctttacaacctaaggcgaggaactgcccttgctattccacaatgt
cgtcttacaccattgagtcgtctcccctttggaatggcccctggacccggcccacaacctggcccgc
taagggagtccattgtctgttatttcatggtctttttacaaactcatatatttgctgaggttttgaa
ggatgcgattaaggaccttgttatgacaaagcccgctcctacctgcaatatcagggtgactgtgtgc
agctttgacgatggagtagatttgcctccctggtttccacctatggtggaaggggctgccgcggagg
gtgatgacggagatgacggagatgaaggaggtgatggagatgagggtgaggaagggcaggagtgatg
taacttgttaggagacgccctcaatcgtattaaaagccgtgtattcccccgcactaaagaataaatc
cccagtagacatcatgcgtgctgttggtgtatttctggccatctgtcttgtcaccattttcgtcctc
ccaacatggggcaattgccggaacccttaatataacttcgtataatgtatgctatacgaagttatta
ggtccctcgaagaggttcactagcggatctcaattgggcatacccatgttgtcacgtcactcagctc
cgcgctcaacaccttctcgcgttggaaaacattagcgacatttacctggtgagcaatcagacatgcg
acggctttagcctggcctccttaaattcacctaagaatgggagcaaccagcaggaaaaggacaagca
gcgaaaattcacgcccccttgggaggtggcggcatatgcaaaggatagcactcccactctactactg
ggtatcatatgctgactgtatatgcatgaggatagcatatgctacccggatacagattaggatagca
tatactacccagatatagattaggatagcatatgctacccagatatagattaggatagcctatgcta
cccagatataaattaggatagcatatactacccagatatagattaggatagcatatgctacccagat
atagattaggatagcctatgctacccagatatagattaggatagcatatgctacccagatatagatt
aggatagcatatgctatccagatatttgggtagtatatgctacccagatataaattaggatagcata
tactaccctaatctctattaggatagcatatgctacccggatacagattaggatagcatatactacc
cagatatagattaggatagcatatgctacccagatatagattaggatagcctatgctacccagatat
aaattaggatagcatatactacccagatatagattaggatagcatatgctacccagatatagattag
gatagcctatgctacccagatatagattaggatagcatatgctatccagatatttgggtagtatatg
ctacccatggcaacattagcccaccgtgctctcagcgacctcgtgaatatgaggaccaacaaccctg
tgcttggcgctcaggcgcaagtgtgtgtaatttgtcctccagatcgcagcaatcgcgcccctatctt
ggcccgcccacctacttatgcaggtattccccggggtgccattagtggttttgtgggcaagtggttt
gaccgcagtggttagcggggttacaatcagccaagttattacacccttattttacagtccaaaaccg
cagggcggcgtgtgggggctgacgcgtgcccccactccacaatttcaaaaaaaagagtggccacttg
tctttgtttatgggccccattggcgtggagccccgtttaattttcgggggtgttagagacaaccagt
ggagtccgctgctgtcggcgtccactctctttccccttgttacaaatagagtgtaacaacatggttc
acctgtcttggtccctgcctgggacacatcttaataaccccagtatcatattgcactaggattatgt
gttgcccatagccataaattcgtgtgagatggacatccagtctttacggcttgtccccaccccatgg
atttctattgttaaagatattcagaatgtttcattcctacactagtatttattgcccaaggggtttg
tgagggttatattggtgtcatagcacaatgccaccactgaaccccccgtccaaattttattctgggg
gcgtcacctgaaaccttgttttcgagcacctcacatacaccttactgttcacaactcagcagttatt
ctattagctaaacgaaggagaatgaagaagcaggcgaagattcaggagagttcactgcccgctcctt
gatcttcagccactgcccttgtgactaaaatggttcactaccctcgtggaatcctgaccccatgtaa
ataaaaccgtgacagctcatggggtgggagatatcgctgttccttaggacccttttactaaccctaa
ttcgatagcatatgcttcccgttgggtaacatatgctattgaattagggttagtctggatagtatat
actactacccgggaagcatatgctacccgtttagggttaacaagggggccttataaacactattgct
aatgccctcttgagggtccgcttatcggtagctacacaggcccctctgattgacgttggtgtagcct
cccgtagtcttcctgggcccctgggaggtacatgtcccccagcattggtgtaagagcttcagccaag
agttacacataaaggcaatgttgtgttgcagtccacagactgcaaagtctgctccaggatgaaagcc
actcagtgttggcaaatgtgcacatccatttataaggatgtcaactacagtcagagaacccctttgt
gtttggtccccccccgtgtcacatgtggaacagggcccagttggcaagttgtaccaaccaactgaag
ggattacatgcactgccccgcgaagaaggggcagagatgtcgtagtcaggtttagttcgtccggggc
ggggcatcgatcctctagagtcgacgctagcggatccgctgcattaatgaatcggccaacgcgcggg
gagaggcggtttgcgtattgggcgctcttccgcttcctcgctcactgactcgctgcgctcggtcgtt
cggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgct
ggcgtttttccataggctccgcccccctgacgagcatcacaaaaatcgacgctcaagtcagaggtgg
cgaaacccgacaggactataaagataccaggcgtttccccctggaagctccctcgtgcgctctcctg
ttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaa
ccccccgttcagcccgaccgctgcgccttatccggtaactatcgtcttgagtccaacccggtaagac
acgacttatcgccactggcagcagccactggtaacaggattagcagagcgaggtatgtaggcggtgc
tacagagttcttgaagtggtggcctaactacggctacactagaagaacagtatttggtatctgcgct
ctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcc
tttgatcttttctacggggtctgacgctcagtggaacgaaaactcacgttaagggattttggtcatg
agattatcaaaaaggatcttcacctagatccttttaaattaaaaatgaagttttaaatcaatctaaa
gtatatatgagtaaacttggtctgacagttaccaatgcttaatcagtgaggcacctatctcagcgat
ctgtctatttcgttcatccatagttgcctgactccccgtcgtgtagataactacgatacgggagggc
ttaccatctggccccagtgctgcaatgataccgcgagacccacgctcaccggctccagatttatcag
caataaaccagccagccggaagggccgagcgcagaagtggtcctgcaactttatccgcctccatcca
gtctattaattgttgccgggaagctagagtaagtagttcgccagttaatagtttgcgcaacgttgtt
gccattgctacaggcatcgtggtgtcacgctcgtcgtttggtatggcttcattcagctccggttccc
aacgatcaaggcgagttacatgatcccccatgttgtgcaaaaaagcggttagctccttcggtcctcc
gatcgttgtcagaagtaagttggccgcagtgttatcactcatggttatggcagcactgcataattct
cttactgtcatgccatccgtaagatgcttttctgtgactggtgagtactcaaccaagtcattctgag
aatagtgtatgcggcgaccgagttgctcttgcccggcgtcaatacgggataataccgcgccacatag
cagaactttaaaagtgctcatcattggaaaacgttcttcggggcgaaaactctcaaggatcttaccg
ctgttgagatccagttcgatgtaacccactcgtgcacccaactgatcttcagcatcttttactttca
ccagcgtttctgggtgagcaaaaacaggaaggcaaaatgccgcaaaaaagggaataagggcgacacg
gaaatgttgaatactcatactcttcctttttcaatattattgaagcatttatcagggttattgtctc
atgagcggatacatatttgaatgtatttagaaaaataaacaaataggggttccgcgcacatttcccc
gaaaagtgccacctggg
```

In SEQ ID NO: 199, the bolded. double underlined text represents the sequence of miR-302s, and the bolded, underlined text represents the sequence of miRNA-200c.

### REFERENCES

1. Warburg, 0., Wind, F. & Negelein, E. The Metabolism of Tumors in the Body. J Gen Physiol 8, 519-530 (1927).
2. Warburg, 0. On the origin of cancer cells. Science 123, 309-314 (1956).
3. Rafalski, V. A., Mancini, E. & Brunet, A. Energy metabolism and energy-sensing pathways in mammalian embryonic and adult stem cell fate. Journal of cell science 125, 5597-5608 (2012).
4. Ito, K. & Suda, T. Metabolic requirements for the maintenance of self-renewing stem cells. Nature reviews. Molecular cell biology 15, 243-256 (2014).
5. Burgess, R. J., Agathocleous, M. & Morrison, S. J. Metabolic regulation of stem cell function. J Intern Med 276, 12-24 (2014).
6. Zhang, J., Nuebel, E., Daley, G. Q., Koehler, C. M. & Teitell, M. A. Metabolic regulation in pluripotent stem cells during reprogramming and self-renewal. Cell stem cell 11, 589-595 (2012).
7. Folmes, C. D., Dzeja, P. P., Nelson, T. J. & Terzic, A. Metabolic plasticity in stem cell homeostasis and differentiation. Cell stem cell 11, 596-606 (2012).
8. Zhang, J et al. UCP2 regulates energy metabolism and differentiation potential of human pluripotent stem cells. The EMBO journal 30, 4860-4873 (2011).
9. Zhou, W. et al. HIF 1 alpha induced switch from bivalent to exclusively glycolytic metabolism during ESC-to-EpiSC/hESC transition. EMBO J 31, 2103-2116 (2012).
10. Varum, S. et al. Energy metabolism in human pluripotent stem cells and their differentiated counterparts. PLoS One 6, e20914 (2011).
11. Folmes, C. D. et al. Somatic oxidative bioenergetics transitions into pluripotency-dependent glycolysis to facilitate nuclear reprogramming. Cell metabolism 14, 264-271 (2011).
12. Moussaieff, A. et al. Glycolysis-Mediated Changes in Acetyl-CoA and Histone Acetylation Control the Early Differentiation of Embryonic Stem Cells. Cell metabolism 21, 392-402 (2015).
13. Panopoulos, A. D. et al. The metabolome of induced pluripotent stem cells reveals metabolic changes occurring in somatic cell reprogramming. Cell research 22, 168-177 (2012).
14. Kim, S. C. et al. Substrate and functional diversity of lysine acetylation revealed by a proteomics survey. Mol Cell 23, 607-618 (2006).
15. Zhao, S. et al. Regulation of cellular metabolism by protein lysine acetylation. Science 327, 1000-1004 (2010).
16. Wang, Q. et al. Acetylation of metabolic enzymes coordinates carbon source utilization and metabolic flux. Science 327, 1004-1007 (2010).
17. Guarente, L. The logic linking protein acetylation and metabolism. Cell metabolism 14, 151-153 (2011).
18. Guarente, L. Franklin H. Epstein Lecture: Sirtuins, aging, and medicine. The New England journal of medicine 364, 2235-2244 (2011).
19. Finkel, T., Deng, C. X. & Mostoslaysky, R. Recent progress in the biology and physiology of sirtuins. Nature 460, 587-591 (2009).
20. Zhang, J. et al. Measuring energy metabolism in cultured cells, including human pluripotent stem cells and differentiated cells. Nature protocols 7, 1068-1085 (2012).
21. Gatenby, R. A. & Gillies, R. J. Why do cancers have high aerobic glycolysis? Nat Rev Cancer 4, 891-899 (2004).
22. Mathieu, J. et al. Hypoxia-Inducible Factors Have Distinct and Stage-Specific Roles during Reprogramming of Human Cells to Pluripotency. Cell stem cell 14, 592-605 (2014).
23. Prigione, A. et al. HIF 1 alpha modulates cell fate reprogramming through early glycolytic shift and upregulation of PDK1-3 and PKM2. Stem Cells 32, 364-376 (2014).
24. North, B. J., Marshall, B. L., Borra, M. T., Denu, J. M. & Verdin, E. The human Sir2 ortholog, SIRT2, is an NAD+-dependent tubulin deacetylase. Mol Cell 11, 437-444 (2003).
25. Lynn, E. G., McLeod, C. J., Gordon, J. P., Bao, J. & Sack, M. N. SIRT2 is a negative regulator of anoxia-reoxygenation tolerance via regulation of 14-3-3 zeta and BAD in H9c2 cells. FEBS Lett 582, 2857-2862 (2008).
26. Fathi, A. et al. Comprehensive gene expression analysis of human embryonic stem cells during differentiation into neural cells. PLoS One 6, e22856 (2011).
27. Assou, S. et al. A meta-analysis of human embryonic stem cells transcriptome integrated into a web-based expression atlas. Stem Cells 25, 961-973 (2007).
28. Li, Z. et al. Functional and transcriptional characterization of human embryonic stem cell-derived endothelial cells for treatment of myocardial infarction. PLoS One 4, e8443 (2009).
29. Masaki, H. et al. Heterogeneity of pluripotent marker gene expression in colonies generated in human iPS cell induction culture. Stem Cell Res 1, 105-115 (2007).
30. Barrett, T. et al. NCBI GEO: archive for functional genomics data sets—update. Nucleic Acids Res 41, D991-995 (2013).
31. Calvanese, V. et al. Sirtuin 1 regulation of developmental genes during differentiation of stem cells. Proc Nall Acad Sci USA 107, 13736-13741 (2010).
32. Lee, Y. L. et al. Sirtuin 1 facilitates generation of induced pluripotent stem cells from mouse embryonic fibroblasts through the miR-34a and p53 pathways. PloS one 7, e45633 (2012).
33. Si, X. et al. Activation of GSK3beta by Sirt2 is required for early lineage commitment of mouse embryonic stem cell. PloS one 8, e76699 (2013).
34. Xiong, Y. & Guan, K. L. Mechanistic insights into the regulation of metabolic enzymes by acetylation. J Cell Biol 198, 155-164 (2012).
35. Marchler-Bauer, A. et al. CDD: NCBI's conserved domain database. Nucleic acids research 43, D222-226 (2015).
36. Gamblin, S. J. et al. Activity and specificity of human aldolases. Journal of molecular biology 219, 573-576 (1991).
37. Park, K. S. et al. Transcription elongation factor Tcea3 regulates the pluripotent differentiation potential of mouse embryonic stem cells via the Lefty1-Nodal-Smad2 pathway. Stem Cells 31, 282-292 (2013).
38. Ivanova, N et al. Dissecting self-renewal in stem cells with RNA interference. Nature 442, 533-538 (2006).
39. Mu, W. L. et al. Sox2 Deacetylation by Sirtl Is Involved in Mouse Somatic Reprogramming. Stem Cells 33, 2135-2147 (2015).
40. Miranda, K. C. et al. A pattern-based method for the identification of MicroRNA binding sites and their corresponding heteroduplexes. Cell 126, 1203-1217 (2006).
41. Suh, M. R. et al. Human embryonic stem cells express a unique set of microRNAs. Developmental biology 270, 488-498 (2004).
42. Wang, G. et al. Critical regulation of miR-200/ZEB2 pathway in Oct4/Sox2-induced mesenchymal-to-epithelial transition and induced pluripotent stem cell generation. Proceedings of the National Academy of Sciences of the United States of America 110, 285 8-2863 (2013).
43. Gomes, P., Outeiro, T. F. & Cavadas, C. Emerging Role of Sirtuin 2 in the Regulation of Mammalian Metabolism. Trends in pharmacological sciences 36, 756-768 (2015).
44. Hallows, W. C., Yu, W. & Denu, J. M. Regulation of glycolytic enzyme phosphoglycerate mutase-1 by Sirtl protein-mediated deacetylation. The Journal of biological chemistry 287, 3850-3858 (2012).
45. Tsusaka, T. et al. Deacetylation of phosphoglycerate mutase in its distinct central region by SIRT2 down-regulates its enzymatic activity. Genes Cells 19, 766-777 (2014).
46. Xu, Y. et al. Oxidative stress activates SIRT2 to deacetylate and stimulate phosphoglycerate mutase. Cancer Res 74, 3630-3642 (2014).
47. Li, T. et al. Glyceraldehyde-3-phosphate dehydrogenase is activated by lysine 254 acetylation in response to glucose signal. J Biol Chem 289, 3775-3785 (2014).
48. Somers, A. et al. Generation of transgene-free lung disease-specific human induced pluripotent stem cells using a single excisable lentiviral stem cell cassette. Stem Cells 28, 1728-1740 (2010).
49. Takahashi, K. et al. Induction ofpluripotent stem cells from adult human fibroblasts by defined factors. Cell 131, 861-872 (2007).
50. Kislinger, T. et al. Global survey of organ and organelle protein expression in mouse: combined proteomic and transcriptomic profiling. Cell 125, 173-186 (2006).
51. Zhou, J. Y. et al. Galectin-3 is a candidate biomarker for amyotrophic lateral sclerosis: discovery by a proteomics approach. Journal of proteome research 9, 5133-5141 (2010).
52. Kim, D. et al. Generation of human induced pluripotent stem cells by direct delivery of reprogramming proteins. Cell stem cell 4, 472-476 (2009).

TABLE 2
List of hyperacetylated proteins
in hESCs includes five glycolytic enzymes.

| | Acession | Molec. | hDF | hESC | hESC/ | | | Representative | Seq |
|---|---|---|---|---|---|---|---|---|---|
| Description | No. | Weight | peptide | peptide | hDF | G-stat | p-value | peptide | ID |
| Fatty acid | IPI00026781 | 273 kDa | 3 | 34 | 11.33 | 30.46916 | 3.39E-08 | (R)FPQLDSTSFAN | SEQ |
| synthase | | | | | | | | SR(D) | ID |
| | | | | | | | | | NO: |
| | | | | | | | | | 30 |
| Fructose- | IPI00418262 | 48 kDa | 1 | 10 | 10 | 8.547244 | 0.003460458 | (R)YASIQQGIVPI | SEQ |
| bisphosphate | | | | | | | | VEPEILPDGDHDLK | ID |
| aldolase | | | | | | | | (R) | NO: |
| | | | | | | | | | 31 |
| Ubiquitin-like | IPI00645078 | 118 kDa | 1 | 10 | 10 | 8.547244 | 0.003460458 | (R)YDGQVAVFGSD | SEQ |
| modifier- | | | | | | | | LQEK(L) | ID |
| activating | | | | | | | | | NO: |
| enzyme 1 | | | | | | | | | 32 |
| ATP synthase | IPI00303476 | 57 kDa | 2 | 18 | 9 | 14.72257 | 0.000124547 | (K)TVLIELINVAK | SEQ |
| subunit beta, | | | | | | | | (A) | ID |
| mitochondrial | | | | | | | | | NO: |
| | | | | | | | | | 33 |
| Isoform alpha- | IPI00465248 | 47 kDa | 2 | 13 | 6.5 | 9.014181 | 0.002678928 | (K)VNQIGSVTESI | SEQ |
| enolase | | | | | | | | QAK(L) | ID |
| | | | | | | | | | NO: |
| | | | | | | | | | 34 |
| Phospho- | IPI00169383 | 45 kDa | 4 | 21 | 5.25 | 12.67387 | 0.000370802 | (R)AHSSVGVNLPQ | SEQ |
| glycerate | | | | | | | | K(A) | ID |
| kinase 1 | | | | | | | | | NO: |
| | | | | | | | | | 35 |
| Actin, | IPI00021439 | 42 kDa | 4 | 17 | 4.25 | 8.661848 | 0.003249416 | (K)DSYVGDEAQSK | SEQ |
| cytoplasmic 1 | | | | | | | | (R) | ID |
| | | | | | | | | | NO: |
| | | | | | | | | | 36 |
| Transitional | IPI00022774 | 89 kDa | 5 | 20 | 4 | 9.637238 | 0.001906718 | (R)IVSQLLTLMDG | SEQ |
| endoplasmic | | | | | | | | LK(Q) | ID |
| reticulum | | | | | | | | | NO: |
| ATPase | | | | | | | | | 37 |
| 14-3-3 | IPI00021263 | 28 kDa | 4 | 15 | 3.75 | 6.782773 | 0.009204181 | (R)YLAEVAAGDDK | SEQ |
| protein | | | | | | | | (K) | ID |
| zeta/delta | | | | | | | | | NO: |
| | | | | | | | | | 38 |
| Isoform 1 | IPI00003865 | 71 kDa | 7 | 26 | 3.71 | 11.64198 | 0.0006448 | (K)NQVAMNPTNTV | SEQ |
| of Heat | | | | | | | | FDAK(R) | ID |
| shock cognate | | | | | | | | | NO: |
| 71 kDa protein | | | | | | | | | 39 |
| Protein | IPI00009904 | 73 kDa | 4 | 11 | 2.75 | 3.39696 | 0.065316682 | (K)VEGFPTIYFAP | SEQ |
| disulfide- | | | | | | | | SGDK(K) | ID |
| isomerase A4 | | | | | | | | | NO: |
| | | | | | | | | | 40 |
| Heat shock | IPI00414676 | 83 kDa | 15 | 40 | 2.67 | 11.7914 | 0.000595049 | (R)TLTIVDTGIGM | SEQ |
| protein HSP | | | | | | | | TK(A) | ID |
| 90-beta | | | | | | | | | NO: |
| | | | | | | | | | 41 |
| Isoform 1 of | IPI00784295 | 85 kDa | 7 | 16 | 2.29 | 3.617618 | 0.057170688 | (K)HSQFIGYPITL | SEQ |
| Heat shock | | | | | | | | FVEK(E) | ID |
| protein | | | | | | | | | NO: |
| HSP 90-alpha | | | | | | | | | 42 |
| Isoform Long of | IPI00883857 | 91 kDa | 6 | 13 | 2.17 | 2.640709 | 0.104157079 | (R)GYFEYIEENK | SEQ |
| Heterogeneous | | | | | | | | (Y) | ID |
| nuclear | | | | | | | | | NO: |
| ribonucleo- | | | | | | | | | 43 |
| protein U | | | | | | | | | |
| Tubulin alpha- | IPI00180675 | 50 kDa | 9 | 19 | 2.11 | 3.651513 | 0.056018296 | (K)TIGGGDDSFNT | SEQ |
| 1A chain | | | | | | | | FFSETGAGK(H) | ID |
| | | | | | | | | | NO: |
| | | | | | | | | | 44 |
| Tubulin beta- | IPI00007752 | 50 kDa | 13 | 27 | 2.08 | 5.005292 | 0.025269938 | (R)IMNTFSVVPSP | SEQ |
| 2C chain | | | | | | | | K(V) | ID |
| | | | | | | | | | NO: |
| | | | | | | | | | 45 |
| 60 kDa | IPI00784154 | 61 kDa | 5 | 10 | 2 | 1.69899 | 0.192420068 | (K)VGGTSDVEVNE | SEQ |
| heat shock | | | | | | | | K(K) | ID |
| protein, | | | | | | | | | NO: |
| mitochondrial | | | | | | | | | 46 |
| Pyruvate | IPI00220644 | 57 kDa | 5 | 10 | 2 | 1.69899 | 0.192420068 | (R)LNFSHGTHEYH | SEQ |
| kinase M1/M2 | | | | | | | | AETIK(N) | ID |
| | | | | | | | | | NO: |
| | | | | | | | | | 47 |
| Heat shock | IPI00304925 | 70 kDa | 7 | 13 | 1.86 | 1.828022 | 0.176361396 | (K)NQVALNPQNTV | SEQ |
| 70 kDa | | | | | | | | FDAK(R) | ID |
| protein 1A/1B | | | | | | | | | NO: |
| | | | | | | | | | 48 |
| Fructose- | IPI00465439 | 39 kDa | 9 | 16 | 1.78 | 1.986449 | 0.158712633 | (K)GILAADESTGS | SEQ |
| bisphosphate | | | | | | | | IAK(R) | ID |
| aldolase A | | | | | | | | | NO: |
| | | | | | | | | | 49 |
| Glyceraldehyde- | IPI00219018 | 36 kDa | 9 | 16 | 1.78 | 1.986449 | 0.158712633 | (R)GALQNIIPAST | SEQ |
| 3-phosphate | | | | | | | | GAAK(A) | ID |
| dehydrogenase | | | | | | | | | NO: |
| | | | | | | | | | 50 |
| 78 kDa glucose- | IPI00003362 | 72 kDa | 15 | 24 | 1.6 | 2.095762 | 0.147708093 | (R)IINEPTAAAIA | SEQ |
| regulated | | | | | | | | YGLDK(R) | ID |
| protein | | | | | | | | | NO: |
| | | | | | | | | | 51 |
| Hydroxy- | IPI00008475 | 57 kDa | 0 | 14 | | 19.40812 | 1.06E-05 | (K)VTQDATPGSAL | SEQ |
| methylglutaryl- | | | | | | | | DK(I) | ID |
| CoA synthase, | | | | | | | | | NO: |
| cytoplasmic | | | | | | | | | 52 |
| THO complex | IPI00328840 | 28 kDa | 0 | 13 | | 18.02183 | 2.18E-05 | (R)SLGTADVHFER | SEQ |
| subunit 4 | | | | | | | | (K) | ID |
| | | | | | | | | | NO: |
| | | | | | | | | | 53 |
| Nuclease- | IPI00031812 | 36 kDa | 0 | 13 | | 18.02183 | 2.18E-05 | (K)EDVFVHQTAIK | SEQ |
| sensitive | | | | | | | | (K) | ID |
| element-binding | | | | | | | | | NO: |
| protein 1 | | | | | | | | | 54 |
| Insulin-like | IPI00008557 | 63 kDa | 0 | 11 | | 15.24924 | 9.42E-05 | (R)MVIITGPPEAQ | SEQ |
| growth factor 2 | | | | | | | | FK(A) | ID |
| RNAbinding | | | | | | | | | NO: |
| protein 1 | | | | | | | | | 55 |
| Isoform 1 of | IPI00219526 | 61 kDa | 0 | 11 | | 15.24924 | 9.42E-05 | (K)FNISNGGPAPE | SEQ |
| Phospho- | | | | | | | | AITDK(I) | ID |
| glucomutase-1 | | | | | | | | | NO: |
| | | | | | | | | | 56 |
| Isocitrate | IPI00027223 | 47 kDa | 0 | 10 | | 13.86294 | 0.000196638 | (K)VEITYTPSDGT | SEQ |
| dehydrogenase | | | | | | | | QK(V) | ID |
| [NADP] | | | | | | | | | NO: |
| cytoplasmic | | | | | | | | | 57 |

TABLE 3
List of hypoacetylated proteins in hESCs.

| | Acession | Molec. | hDF | hESC | hESC/ | | | Representative | |
|---|---|---|---|---|---|---|---|---|---|
| Description | No. | Weight | peptide | peptide | hDF | G-stat | p-value | peptide | |
| Talin-1 | IPI00298994 | 270 kDa | 11 | 17 | 0.65 | 1.295739 | 0.254993 | (R)ILAQATSDLVN | SEQ ID |
| | | | | | | | | AIK(A) | NO: 58 |
| Actinin alpha 1 | IPI00921118 | 107 kDa | 7 | 11 | 0.64 | 0.896353 | 0.343761 | (K)VLAVNQENEQL | SEQ ID |
| isoform 3 | | | | | | | | MEDYEK(L) | NO: 59 |
| Isoform 1 of | IPI00022418 | 263 kDa | 13 | 23 | 0.57 | 2.814651 | 0.093407 | (K)WCGTTQNYDAD | SEQ ID |
| Fibronectin | | | | | | | | QK(F) | NO: 60 |
| Isoform 2 of | IPI00418169 | 40 kDa | 6 | 11 | 0.55 | 1.49256 | 0.22182 | (K)LSLEGDHSTPP | SEQ ID |
| Annexin A2 | | | | | | | | SAYGSVK(A) | NO: 61 |
| Non-POU domain- | IPI00304596 | 54 kDa | 6 | 12 | 0.5 | 2.038788 | 0.153332 | (R)PVTVEPMDQLD | SEQ ID |
| containing | | | | | | | | DEEGLPEK(L) | NO: 62 |
| octamer- | | | | | | | | | |
| binding protein | | | | | | | | | |
| Isoform A1-A of | IPI00465365 | 34 kDa | 3 | 9 | 0.33 | 3.139489 | 0.076418 | (R)EDSQRPGAHLT | SEQ ID |
| Heterogeneous | | | | | | | | VK(K) | NO: 63 |
| nuclear ribo- | | | | | | | | | |
| nucleoprotein A1 | | | | | | | | | |
| Isoform 1 of | IPI00019502 | 227 kDa | 6 | 25 | 0.24 | 12.51282 | 0.000404 | (R)LTEMETLQSQL | SEQ ID |
| Myosin-9 | | | | | | | | MAEK(L) | NO: 64 |
| Annexin A6 | IPI00221226 | 76 kDa | 2 | 9 | 0.22 | 4.818173 | 0.028161 | (R)PANDFNPDADA | SEQ ID |
| | | | | | | | | K(A) | NO: 65 |
| Isoform B1 of | IPI00396378 | 37 kDa | 2 | 9 | 0.22 | 4.818173 | 0.028161 | (R)EESGKPGAHVT | SEQ ID |
| Heterogeneous | | | | | | | | VK(K) | NO: 66 |
| nuclear ribo- | | | | | | | | | |
| nucleoproteins | | | | | | | | | |
| A2/B1 | | | | | | | | | |
| p180/ribosome | IPI00856098 | 166 kDa | 4 | 19 | 0.21 | 10.63107 | 0.001112 | (K)LLATEQEDAAV | SEQ ID |
| receptor | | | | | | | | AK(S) | NO: 67 |
| Cytoskeleton- | IPI00141318 | 66 kDa | 5 | 24 | 0.21 | 13.54034 | 0.000233 | (K)SINDNIAIFTE | SEQ ID |
| associated | | | | | | | | VQK(R) | NO: 68 |
| protein 4 | | | | | | | | | |
| Isoform A of | IPI00021405 | 74 kDa | 4 | 32 | 0.13 | 24.79069 | 6.39E-07 | (K)AAYEAELGDAR | SEQ ID |
| Prelamin-A/C | | | | | | | | (K) | NO: 69 |
| Isoform 1 of | IPI00022200 | 344 kDa | 1 | 13 | 0.08 | 12.2032 | 0.000477 | (K)SDDEVDDPAVE | SEQ ID |
| Collagen | | | | | | | | LK(Q) | NO: 70 |
| alpha-3(VI) | | | | | | | | | |
| chain | | | | | | | | | |
| Isoform 2 of | IPI00413958 | 287 kDa | 1 | 29 | 0.03 | 32.82015 | 1.01E-08 | (K)GAGTGGLGLTV | SEQ ID |
| Filamin-C | | | | | | | | EGPcEAK(I) | NO: 71 |
| Neuroblast | IPI00021812 | 629 kDa | 0 | 21 | | 29.11218 | 6.83E-08 | (R)FPQLDSTSFAN | SEQ ID |
| differentiation- | | | | | | | | SR(D) | NO: 72 |
| associated | | | | | | | | | |
| protein AHNAK | | | | | | | | | |
| Talin-1 | IPI00298994 | 270 kDa | 11 | 17 | 0.65 | 1.295739 | 0.254993 | (R)ILAQATSDLVN | SEQ ID |
| | | | | | | | | AIK(A) | NO: 73 |

TABLE 4a
Meta-analyses of hPSCs and their differentiated cells. hESCs, hiPSCs, and their differentiated cells were grouped and
meta-analysis for HAT family was performed using GEO2R. The meta-analysis did not reveal any change in HAT
expression pattern in hESC and hiPSC. GEO accession numbers GSE28633, GSE18265, GSE20013, GSE39144, and
GSE9709 were used for the analysis. Adj. P. Val indicates P-value adjustment for mutiple comparisons.

| | | adj | | Gene | | | | |
|---|---|---|---|---|---|---|---|---|
| GSE# | ID | P. Val | P Value | symbol | Gene title | Expression | Samples | Ref. |
| GSE28633 | ILMN_2095840 | 4.24E−03 | 4.37E−04 | KAT6A | K(lysine) | Down | 3 hESCs and 3 | 30 |
| | | | | | acetyltransferase 6A | | Neural cells | |
| | ILMN_1725244 | 5.27E−02 | 9.20E−03 | HAT1 | histone | Up | | |
| | | | | | acetyltransferase 1 | | | |
| | ILMN_2293692 | 8.25E−02 | 1.59E−02 | CREBBP | CREB binding | Up | | |
| | | | | | protein (CBP) | | | |
| | ILMN_1782247 | 9.32E−02 | 1.85E−02 | KAT2A | K(lysine) | Up | | |
| | | | | | acetyltransferase 2A | | | |
| | | | | | (GCN5) | | | |
| GSE18265 | None | | | | | | 4 hESCs, 3 hiPSCs | 31 |
| | | | | | | | and 1 hFF | |
| GSE20013 | A_23_P339480 | 5.73E−07 | 7.12E−08 | HAT1 | histone | Up | 4 hESCs, 4 ECs | 32 |
| | | | | | acetyltransferase 1 | | (hESCs) and 4 | |
| | A_32_P159651 | 3.39E−06 | 6.09E−07 | KAT2B | K(lysine) | Down | HUVECs | |
| | | | | | acetyltransferase 2B | | | |
| | A_24_P941586 | 5.38E−06 | 1.05E−06 | KAT6B | K(lysine) | Up | | |
| | | | | | acetyltransferase 6B | | | |
| GSE39144 | 226547_at | 2.53E−08 | 1.97E−10 | KAT6A | K(lysine) | Down | 3 hESCs, 6 | Unpublished |
| | | | | | acetyltransferase 6A | | hiPSCs, 4 Neurons | |
| | 203845_at | 1.18E−07 | 1.89E−09 | KAT2B | K(lysine) | Down | (hESCs), 7 | |
| | | | | | acetyltransferase 2B | | Neurons (hiPSCs) | |
| | 202423_at | 2.00E−06 | 8.79E−08 | KAT6A | K(lysine) | Down | and 1 hDF | |
| | | | | | acetyltransferase 6A | | | |
| | 239585_at | 3.02E−06 | 1.54E−07 | KAT2B | K(lysine) | Down | | |
| | | | | | acetyltransferase 2B | | | |
| GSE9709 | 203845_at | 0.0139 | 2.98E−04 | KAT2B | K(lysine) | Down | 6 hiPSCs and 2 | 33 |
| | | | | | acetyltransferase 2B | | hDFs | |
| | 1559142_at | 0.23203 | 4.59E−02 | KAT6A | K(lysine) | Down | | |
| | | | | | acetyltransferase 6A | | | |

TABLE 4b
Meta-analyses of hPSCs and their differentiated cells.
Compiled HAT family data used in this study. Expression
levels of each HAT family member shown as up, down,
and N/A indicate up-regulated, down-regulated, and no
significant change respectively in hESCs. Numbers it
parentheses indicate the number of changed expression
among the 5 different studies.

| Gene | | Expression in hESCs |
|---|---|---|
| Symbol | Gene Title | (# of studies) |
| HAT1 | histone acetyltransferase 1 | Up (1) |
| KAT2A | K(lysine) acetyltransferase 2A | Up (1) |
| | (GCN5) | |
| KAT2B | K(lysine) acetyltransferase 2B | Down (3) |
| KAT5 | K(lysine) acetyltransferase 5 | N/A |
| KAT6A | K(lysine) acetyltransferase 6A | Down (3) |
| KAT6B | K(lysine) acetyltransferase 6B | Up (1) |
| KAT7 | K(lysine) acetyltransferase 7 | N/A |
| KAT8 | K(lysine) acetyltransferase 8 | N/A |

TABLE 5a
Meta-analyses of HDAC family gene expression. hESCs, hiPSCs, and their differentiated cells
were grouped and meta-analyses performed by GEO2R for HDAC family gene expression.

| | | adj | | | | | | |
|---|---|---|---|---|---|---|---|---|
| GSE# | ID | P. Val | P Value | Gene symbol | Gene title | Expression | Samples | Ref. |
| GSE28633 | ILMN_1727458 | 2.62E−06 | 5.03E−08 | HDAC1 | histone deacetylase 1 | Up | 3 hESCs and 3 | 30 |
| | ILMN_2398711 | 2.36E−05 | 7.88E−07 | SIRT2 | sirtuin 2 | Down | Neural cells | |
| | ILMN_2291644 | 1.03E−04 | 4.82E−06 | SIRT5 | sirtuin 5 | Down | | |
| | ILMN_1657868 | 1.05E−04 | 4.94E−06 | SIRT4 | sirtuin 4 | Down | | |
| | ILMN_1810856 | 1.48E−04 | 7.55E−06 | HDAC5 | histone deacetylase 5 | Down | | |
| | ILMN_1739083 | 2.71E−04 | 1.55E−05 | SIRT1 | sirtuin 1 | Up | | |
| | ILMN_1683059 | 1.06E−03 | 8.15E−05 | SIRT5 | sirtuin 5 | Up | | |
| | ILMN_1723494 | 1.17E−02 | 1.49E−03 | SIRT2 | sirtuin 2 | Down | | |
| | ILMN_1798546 | 2.96E−02 | 4.55E−03 | HDAC6 | histone deacetylase 6 | Down | | |
| | ILMN_1799598 | 6.10E−02 | 1.10E−02 | SIRT5 | sirtuin 5 | Up | | |
| | ILMN_1772455 | 7.43E−02 | 1.40E−02 | HDAC3 | histone deacetylase 3 | Up | | |
| GSE18265 | 218878_s_at | 0.03419 | 1.31E−03 | SIRT1 | sirtuin 1 | Up | 4 hESCs, 3 hiPSCs | 31 |
| | 220047_at | 0.11623 | 1.18E−02 | SIRT4 | sirtuin 4 | Up | and 1 hFF | |
| | 205659_at | 0.1889 | 3.00E−02 | HDAC9 | histone deacetylase 9 | Up | | |
| | 220605_s_at | 0.20971 | 3.70E−02 | SIRT2 | sirtuin 2 | Down | | |
| GSE20013 | A_23_P122304 | 2.38E−08 | 1.22E−09 | HDAC2 | histone deacetylase 2 | Up | 4 hESCs, 4 ECs | 32 |
| | | | | | | | (hESCs) and 4 | |
| | A_24_P125283 | 7.40E−07 | 9.75E−08 | HDAC5 | histone deacetylase 5 | Up | HUVECs | |
| | A_23_P98022 | 6.75E−09 | 2.11E−10 | SIRT1 | sirtuin 1 | Up | | |
| | A_23_P142455 | 1.99E−06 | 3.24E−07 | SIRT2 | sirtuin 2 | Down | | |
| GSE39144 | 228813_at | 1.62E−05 | 1.38E−06 | HDAC4 | histone deacetylase 4 | Down | 3 hESCs, 6 | Unpublished |
| | 223908_at | 3.25E−06 | 1.70E−07 | HDAC8 | histone deacetylase 8 | Up | hiPSCs, 4 Neurons | |
| | 218878_s_at | 7.66E−06 | 5.29E−07 | SIRT1 | sirtuin 1 | Up | (hESCs), 7 | |
| | 1558331_at | 2.23E−06 | 1.02E−07 | SIRT2 | sirtuin 2 | Down | Neurons (hiPSCs) | |
| | 219185_at | 1.19E−05 | 9.30E−07 | SIRT5 | sirtuin 5 | Up | and 1 hDF | |
| GSE9709 | 232870_at | 0.08736 | 7.98E−03 | HDAC10 | histone deacetylase | Down | 6 hiPSCs and 2 | 33 |
| | | | | | 10 | | hDFs | |
| | 229408_at | 0.13408 | 1.70E−02 | HDAC5 | histone deacetylase 5 | Down | | |
| | 223908_at | 0.21785 | 4.08E−02 | HDAC8 | histone deacetylase 8 | Up | | |
| | 1558331_at | 0.06372 | 4.61E−03 | SIRT2 | sirtuin 2 | Down | | |
| | 220605_s_at | 0.12525 | 1.51E−02 | SIRT2 | sirtuin 2 | Down | | |
| | 222080_s_at | 0.15282 | 2.15E−02 | SIRT5 | sirtuin 5 | N/A | | |
| | 229112_at | 0.18893 | 3.15E−02 | SIRT5 | sirtuin 5 | N/A | | |

TABLE 5b
Compiled data used in this study for HDAC family.
Expression levels of each family member shown as up,
down, and N/A indicate up-regulated, down-regulated, and
no significant change respectively in hESCs. Numbers in
parentheses indicate the number of changed expression
among the 5 different studies.

| Gene | | Expression in hESCs |
|---|---|---|
| Symbol | Gene Title | (# of studies) |
| SIRT1 | sirtuin 1 | Up (4/5) |
| SIRT2 | sirtuin 2 | Down (5/5) |
| SIRT3 | sirtuin 3 | N/A |
| SIRT4 | sirtuin 4 | Up (1)/Down (1) |
| SIRT5 | sirtuin 5 | Up (2)/Down (1) |
| SIRT6 | sirtuin 6 | N/A |
| SIRT7 | sirtuin 7 | N/A |

TABLE 5c
Compiled data used in this study for Sirtuin family.
Expression levels of each family member shown as up,
down, and N/A indicate up regulated, down-regulated, and
no significant change respectively in hESCs. Numbers in
parentheses indicate the number of changed expression
among the 5 different studies.

| Gene | | Expression in hESCs |
|---|---|---|
| Symbol | Gene Title | (# of studies) |
| HDAC1 | histone deacetylase 1 | Up (1) |
| HDAC2 | histone deacetylase 2 | Up (1) |
| HDAC3 | histone deacetylase 3 | Up (1) |
| HDAC4 | histone deacetylase 4 | Down (1) |
| HDAC5 | histone deacetylase 5 | Up (1)/Down (2) |
| HDAC6 | histone deacetylase 6 | Down (1) |
| HDAC7 | histone deacetylase 7 | N/A |
| HDAC8 | histone deacetylase 8 | Up (2) |
| HDAC9 | histone deacetylase 9 | Up (1) |
| HDAC10 | histone deacetylase 10 | Down (1) |
| HDAC11 | histone deacetylase 11 | N/A |

TABLE 6a
List of hESC lines and normal somatic cell lines used
for web-based data analyses of FIG. 1B.

| Embryonic stem cell lines | Normal cells |
|---|---|
| Human embryonic stem cell (H9) | Lung fibroblast cell line WI-38 |
| Human embryonic stem cell (T3) | Embryonic skin fibroblast D551 cell line |
| Human embryonic stem cell (SA01) | Extravillous trophoblast cell line SGHPL-5 |
| Human embryonic stem cell (HD90) | Neonatal foreskin keratinocyte NHEK cell line |
| Human embryonic stem cell (VUB01) | Extravillous trophoblast cell line HTR-8_SVneo |
| Human embryonic stem cell (HS181) | Neonatal melanocyte cell line HEM-N |
| Human embryonic stem cell (WIBR3) | Fibroblast of skin cell line GM-5659 |
| Human embryonic stem cell (HS235) | Umbilical vein cell line HUVEC |
| Human embryonic stem cell (HD129) | Melanocyte cell line Hermes 1 |
| Human embryonic stem cell (HD83) | Melanocyte cell line HEM-LP |
| Human embryonic stem cell (HUES6) | Melanocyte cell line Hermes 2B |
| Human embryonic stem cell (WIBR1) | Breast epithelial cell line HMEC |
| Human embryonic stem cell (Cythera) | Testis fibroblast cell line Hs 1.Tes |
| Human embryonic stem cell (HUES8) | Kidney epithelial cell line HEK-293 |
| Human embryonic stem cell (WIBR2) | Skin keratinocyte HaCaT cell line |
| Human embryonic stem cell (BG01) | |
| Human embryonic stem cell (H7) | |
| Human embryonic stem cell (H14) | |
| Human embryonic stem cell (CSES4) | |
| Human embryonic stem cell (H14A) | |
| Human embryonic stem cell (H13) | |
| Human embryonic stem cell (H13B) | |
| Human embryonic stem cell (ES4) | |
| Human embryonic stem cell (H1) | |
| Human embryonic stem cell (ES2) | |

TABLE 6b
List of originally published data sets for all cell lines used for web-based data analyses.

| GSE# | Description | Platform | Ref. |
|---|---|---|---|
| GSE1822 | Kidney epithelial cell line HEK-293 | Affymetrix Human Genome U133A Array | 52 |
| GSE2638 | Breast epithelial cell line HMEC | Affymetrix Human Genome U133A Array | 53 |
| GSE4975 | Skin keratinocyte cell line HaCaT | Affymetrix Human Genome U133A Array | 54 |
| | | Affymetrix Human Genome U133B Array | |
| | | Affymetrix Human Genome U133 Plus 2.0 Array | |
| GSE7214 | hESCs (SA01, VUB01) | Affymetrix Human Genome U133 Plus 2.0 Array | 55 |
| GSE7216 | Neonatal foreskin keratinocyte cell line NHEK | Affymetrix Human Genome U133 Plus 2.0 Array | 56 |
| GSE9196 | hESCs (H9) | Affymetrix Human Genome U133 Plus 2.0 Array | 57 |
| GSE9440 | hESCs (T3) | Affymetrix Human Genome U133 Plus 2.0 Array | 58 |
| GSE11919 | Fibroblast of skin cell line GM05659 | Affymetrix Human Genome U133 Plus 2.0 Array | 59 |
| GSE12390 | hESCs (HUES8) | Affymetrix Human Genome U133 Plus 2.0 Array | 60 |
| GSE12583 | hESCs (ES2, ES4) | Affymetrix Human Genome U133 Plus 2.0 Array | 61 |
| GSE14711 | hESCs (BG01) | Affymetrix Human Genome U133 Plus 2.0 Array | 62 |
| GSE15148 | hESCs (H1, H7, H13B, H14A) | Affymetrix Human Genome U133 Plus 2.0 Array | 63 |
| GSE15220 | Testis fibroblast cell line Hs 1.Tes | Affymetrix Human Genome U133 Plus 2.0 Array | 64 |
| | | Affymetrix Human Tiling 2.0R Set, Array 1 | |
| | | Affymetrix Human Tiling 2.0R Set, Array 2 | |
| GSE15400 | Embryonic skin fibroblast cell line D551 | Affymetrix Human Genome U133 Plus 2.0 Array | 65 |
| | Lung fibroblast cell line WI-38 | | |
| GSE16654 | hESCs (CSES4) | Affymetrix Human Genome U133 Plus 2.0 Array | 66 |
| | | OSUCCC Human miRNA Expression custom | |
| | | Bioarray | |
| GSE16683 | Umbilical vein cell line HUVEC | Affymetrix Human Genome U133 Plus 2.0 Array | 67 |
| GSE18265 | hESCs (HD83, HD90, HD129, HS181, HS235) | Affymetrix Human Genome U133 Plus 2.0 Array | Unpublished |
| GSE18618 | hESCs (Cythera, HUES6) | Affymetrix Human Genome U133 Plus 2.0 Array | 68 |
| GSE20033 | hESCs (H7, H13, H14) | Affymetrix Human Genome U133 Plus 2.0 Array | 69 |
| GSE20510 | Extravillous trophoblast cell lines (SGHPL-5, HTR-8_Svneo) | Affymetrix Human Genome U133A Array | 70 |
| GSE21222 | hESCs (BG01, WIBR1, WIBR2, WIBR3) | Affymetrix Human Genome U133 Plus 2.0 Array | 71 |
| GSE22167 | hESCs (H1) | Affymetrix Human Genome U133 Plus 2.0 Array | 72 |
| GSE22301 | Melanocyte cell lines (HEM-LP, HEM-N, Hermes 1, Hennes 2B) | Affymetrix Human Genome U133A 2.0 Array | 73 |

TABLE 7
Summary of peptide fragments from acetylated lysine
residues identified from control and SIRT2KD 293T
cells. Symbol @ indicated the site of acetylation
detected by LTQ-Orbitrap mass spectrometry.

| Sample | | Start | End | ModScore | Acetylated | |
|---|---|---|---|---|---|---|
| Name | XCorr | Position | Position | Peptide | Lys | SEQ ID |
| AldoA | 4.841 | 23 | 42 | R.IVAPGK@G | Lys28 | SEQ ID NO: 74 |
| | | | | ILAADESTGS | | |
| | | | | IAK.R | | |
| | 3.014 | 23 | 43 | R.IVAPGKGI | Lys42 | SEQ ID NO: 75 |
| | | | | LAADESTGSI | | |
| | | | | AK@R.L | | |
| | 2.364 | 88 | 101 | K.ADDGRPFP | Lys99 | SEQ ID NO: 76 |
| | | | | QVIK@SK.G | | |
| | 2.961 | 100 | 111 | K.SK@GGVVG | Lys101 | SEQ ID NO: 77 |
| | | | | IKVDK.G | | |
| | 3.205 | 102 | 111 | K.GGVVGIK@ | Lys108 | SEQ ID NO: 78 |
| | | | | VDK.G | | |
| | 2.064 | 141 | 149 | K.DGADFAK@ | Lys147 | SEQ ID NO: 79 |
| | | | | WR.C | | |
| AldoA + | 3.305 | 23 | 42 | R.IVAPGK@G | Lys28 | SEQ ID NO: 80 |
| SIRT2KD | | | | ILAADESTGS | | |
| | | | | IAK.R | | |
| | 4.605 | 29 | 43 | K.GILAADES | Lys42 | SEQ ID NO: 81 |
| | | | | TGSIAK@R.L | | |
| | 2.881 | 88 | 101 | K.ADDGRPFP | Lys99 | SEQ ID NO: 82 |
| | | | | QVIK@SK.G | | |
| | 2.326 | 100 | 111 | K.SK@GGVVG | Lys101 | SEQ ID NO: 83 |
| | | | | IKVDK.G | | |
| | 2.324 | 100 | 111 | K.SKGGVVGI | Lys108 | SEQ ID NO: 84 |
| | | | | K@VDK.G | | |
| | 2.782 | 102 | 111 | K.GGVVGIK@ | Lys108 | SEQ ID NO: 85 |
| | | | | VDK.G | | |
| | 5.024 | 109 | 134 | K.VDK@GVVP | Lys111 | SEQ ID NO: 86 |
| | | | | LAGTNGETTT | | |
| | | | | QGLDGLSER.C | | |
| | 3.101 | 141 | 149 | K.DGADFAK@ | Lys147 | SEQ ID NO: 87 |
| | | | | WR.C | | |
| | 3.189 | 319 | 330 | K.ENLK@AAQ | Lys322 | SEQ ID NO: 88 |
| | | | | EEYVK.R | | |

TABLE 8
List of the predicted MREs on the SIRT2 mRNAs.

| | leftmost | | Folding | | | Base | |
|---|---|---|---|---|---|---|---|
| | position | | energy (in | | | pairs | |
| | of | | Kcal/mol), | | | in | |
| | predicted | | includes | Predicted | Targeting | putative | span |
| | target | cDNA | contribution | target site | miRNA sequence | hetero- | of |
| MiRNA | site | region | from linker | (SEQ ID) | (SEQ ID) | duplex | target |
| hsa_miR_25 | 114 | 5′UTR | -24.9 | AAGCGCGTCTGCGGC | CATTGCACTTGTCTCG | 13 | 22 |
| | | | | CGCAATG | GTCTGA | | |
| | | | | (SEQ ID NO: 89) | (SEQ ID NO: 96) | | |
| hsa_miR_92b | 114 | 5′UTR | -23.799999 | AAGCGCGTCTGCGGC | TATTGCACTCGTCCCG | 14 | 22 |
| | | | | CGCAATG | GCCTCC | | |
| | | | | (SEQ ID NO: 90) | (SEQ ID NO: 97) | | |
| hsa_miR_ | 1416 | CDS | -24.799999 | TCCCCGCCACCTGCC | CGTCTTACCCAGCAGT | 15 | 22 |
| 200c* | | | | AAGGACG | GTTTGG | | |
| | | | | (SEQ ID NO: 91) | (SEQ ID NO: 98) | | |
| | 839 | CDS | -26.6 | ACAGGAGGACTTGGT | CGTCTTACCCAGCAGT | 15 | 22 |
| | | | | GGAGGCG | GTTTGG | | |
| | | | | (SEQ ID NO: 92) | (SEQ ID NO: 99) | | |
| hsa_miR_367 | 133 | 5′UTR | -24.1 | ATGTCTGCTGAGAGT | AATTGCACTTTAGCAA | 15 | 22 |
| | | | | TGTAGTT | TGGTGA | | |
| | | | | (SEQ ID NO: 93) | (SEQ ID NO: 100) | | |
| | 337 | CDS | -23.1 | CCCAGGCAGGGAAGG | AATTGCACTTTAGCAA | 14 | 22 |
| | | | | TGCAGGA | TGGTGA | | |
| | | | | (SEQ ID NO: 94) | (SEQ ID NO: 101) | | |
| | 1084 | CDS | -24 | GTACCTCCTTGCAGG | AATTGCACTTTAGCAA | 16 | 22 |
| | | | | TGCAGCC | TGGTGA | | |
| | | | | (SEQ ID NO: 95) | (SEQ ID NO: 102) | | |

TABLE 9
Sequences of primer used for qRT-PCR analyses and cloning.

| | PCR Primer Sequences (5′ to 3′) | | | |
|---|---|---|---|---|
| Gene | Forward | SEQ ID: | Reverse | SEQ ID |
| SIRT1 | TAGACACGCTGGAACAGGTTGC | (SEQ ID NO: 103) | CTCCTCGTACAGCTTCACAGTC | (SEQ ID NO: 151) |
| SIRT2 | CTGCGGAACTTATTCTCCCAGAC | (SEQ ID NO: 104) | CCACCAAACAGATGACTCTGCG | (SEQ ID NO: 152) |
| SIRT3 | CATTCCAGACTTCAGATCGC | (SEQ ID NO: 105) | AGCAGCCGGAGAAAGTAGT | (SEQ ID NO: 153) |
| SIRT4 | TGGGATCATCCTTGCAGGTAT | (SEQ ID NO: 106) | TGGTCAGCATGGGTCTATCA | (SEQ ID NO: 154) |
| SIRT5 | GCCAAGTTCAAGTATGGCAGA | (SEQ ID NO: 107) | CGCCGGTAGTGGTAGAA | (SEQ ID NO: 155) |
| SIRT6 | TGGCAGTCTTCCAGTGTGGTGT | (SEQ ID NO: 108) | CGCTCTCAAAGGTGGTGTCGAA | (SEQ ID NO: 156) |
| SIRT7 | TGGAGTGTGGACACTGCTTCAG | (SEQ ID NO: 109) | CCGTCACAGTTCTGAGACACCA | (SEQ ID NO: 157) |
| Lmx1b | CAAGGCATCCTTTGAGGTCTC | (SEQ ID NO: 110) | TCCATGCGGCTTGACAGAAC | (SEQ ID NO: 158) |
| Tuj1 | CAACAGCACGGCCATCCAGG | (SEQ ID NO: 111) | CTTGGGGCCCTGGGCCTCCGA | (SEQ ID NO: 159) |
| TH | GAGTACACCGCCGAGGAGATTG | (SEQ ID NO: 112) | GCGGATATACTGGGTGCACTGG | (SEQ ID NO: 160) |
| Oct4 | GCTCGAGAAGGATGTGGTCC | (SEQ ID NO: 113) | CGTTGTGCATAGTCGCTGCT | (SEQ ID NO: 161) |
| Sox2 | AACCCCAAGATGCACAACTC | (SEQ ID NO: 114) | CGGGGCCGGTATTTATAATC | (SEQ ID NO: 162) |
| Nanog | CAAAGGCAAACAACCCACTT | (SEQ ID NO: 115) | TCTGCTGGAGGCTGAGGTAT | (SEQ ID NO: 163) |
| Esrrb | TGTCAAGCCATGATGGAAAA | (SEQ ID NO: 116) | GGTGAGCCAGAGATGCTTTC | (SEQ ID NO: 164) |
| Rex1 | GGCGGAAATAGAACCTGTCA | (SEQ ID NO: 117) | CTTCCAGGATGGGTTGAGAA | (SEQ ID NO: 165) |
| Utf1 | GTCCCCACCGAAGTCTGC | (SEQ ID NO: 118) | GGACACTGTCTGGTCGAAGG | (SEQ ID NO: 166) |
| GDF3 | AAATGTTTGTGTTGCGGTCA | (SEQ ID NO: 119) | TCTGGCACAGGTGTCTTCAG | (SEQ ID NO: 167) |
| Tcl1 | GCCTGGGAGAAGTTCGTGTA | (SEQ ID NO: 120) | ACTAAGCGCCAGAAACTGGA | (SEQ ID NO: 168) |
| Ecat1 | CGAAGGTAGTTCGCCTTGAG | (SEQ ID NO: 121) | CGGTGATAGTCAGCCAGGTT | (SEQ ID NO: 169) |
| Gbx2 | GGTGCAGGTGAAAATCTGGT | (SEQ ID NO: 122) | GCTGCTGATGCTGACTTCTG | (SEQ ID NO: 170) |
| Pax6 | ACCCATTATCCAGATGTGTTTGCCCGAG | (SEQ ID NO: 123) | ATGGTGAAGCTGGGCATAGGCGGCAG | (SEQ ID NO: 171) |
| Map2 | CAGGTGGCGGACGTGTGAAAATTGAGAGTG | (SEQ ID NO: 124) | CACGCTGGATCTGCCTGGGGACTGTG | (SEQ ID NO: 172) |
| GFAP | GGCCCGCCACTTGCAGGAGTACCAGG | (SEQ ID NO: 125) | CTTCTGCTCGGGCCCCTCATGAGACG | (SEQ ID NO: 173) |
| AADC | CGCCAGGATCCCCGCTTTGAAATCTG | (SEQ ID NO: 126) | TCGGCCGCCAGCTCTTTGATGTGTTC | (SEQ ID NO: 174) |
| Foxa2 | TGGGAGCGGTGAAGATGGAAGGGCAC | (SEQ ID NO: 127) | TCATGCCAGCGCCCACGTACGACGAC | (SEQ ID NO: 175) |
| Sox17 | CGCTTTCATGGTGTGGGCTAAGGACG | (SEQ ID NO: 128) | TAGTTGGGGTGGTCCTGCATGTGCTG | (SEQ ID NO: 176) |
| AFP | GAATGCTGCAAACTGACCACGCTGGAAC | (SEQ ID NO: 129) | TGGCATTCAAGAGGGTTTTCAGTCTGGA | (SEQ ID NO: 177) |
| CK8 | CCTGGAAGGGCTGACCGACGAGATCAA | (SEQ ID NO: 130) | CTTCCCAGCCAGGCTCTGCAGCTCC | (SEQ ID NO: 178) |
| CK18 | AGCTCAACGGGATCCTGCTGCACCTTG | (SEQ ID NO: 131) | CACTATCCGGCGGGTGGTGGTCTTTTG | (SEQ ID NO: 179) |
| Msx1 | CGAGAGGACCCCGTGGATGCAGAG | (SEQ ID NO: 132) | GGCGGCCATCTTCAGCTTCTCCAG | (SEQ ID NO: 180) |
| B-T | GCCCTCTCCCTCCCCTCCACGCACAG | (SEQ ID NO: 133) | CGGCGCCGTTGCTCACAGACCACAGG | (SEQ ID NO: 181) |
| Glut1 | TGGCATCAACGCTGTCTTCT | (SEQ ID NO: 134) | AACAGCGACACGACAGTGAA | (SEQ ID NO: 182) |
| Glut2 | GCTGCGAATAAACAGGCAGG | (SEQ ID NO: 135) | AGGGTCCCAGTGACCTTATCT | (SEQ ID NO: 183) |
| Glut3 | GACCCAGAGATGCTGTAATGGT | (SEQ ID NO: 136) | GGGGTGACCTTCTGTGTCCC | (SEQ ID NO: 184) |
| Glut4 | ATTGCTCATGCCCCTACTCA | (SEQ ID NO: 137) | CCTGGTGAAGAGTGCCCCTA | (SEQ ID NO: 185) |
| Glut5 | GCATGAAGGAAGGGAGGCTG | (SEQ ID NO: 138) | ACAGACCACAGCAACGTCAA | (SEQ ID NO: 186) |
| Glut6 | TCTCAGCGGCCATCATGTTT | (SEQ ID NO: 139) | GGCGTAGCCCATGATGAAGA | (SEQ ID NO: 187) |
| Glut7 | CATTCCATTGGGCCCAGTCCT | (SEQ ID NO: 140) | TGAAACTGTAGGCACCGATGG | (SEQ ID NO: 188) |
| AldoA | CAGGGACAAATGGCGAGACTA | (SEQ ID NO: 141) | GGGGTGTGTTCCCCAATCTT | (SEQ ID NO: 189) |
| AldoB | TGTCTGGTGGCATGAGTGAAG | (SEQ ID NO: 142) | GGCCCGTCCATAAGAGAAACTT | (SEQ ID NO: 190) |
| AldoC | GCCAAATTGGGGTGGAAAACA | (SEQ ID NO: 143) | TTCACACGGTCATCAGCACTG | (SEQ ID NO: 191) |
| ENO1 | GCCGTGAACGAGAAGTCCTG | (SEQ ID NO: 144) | ACGCCTGAAGAGACTCGGT | (SEQ ID NO: 192) |
| ENO2 | CCGGGAACTCAGACCTCATC | (SEQ ID NO: 145) | CTCTGCACCTAGTCGCATGG | (SEQ ID NO: 193) |
| ENO3 | TATCGCAATGGGAAGTACGATCT | (SEQ ID NO: 146) | AAGCTCTTATACAGCTCTCCGA | (SEQ ID NO: 194) |
| PGK1 | GAACAAGGTTAAAGCCGAGCC | (SEQ ID NO: 147) | GTGGCAGATTGACTCCTACCA | (SEQ ID NO: 195) |
| PGK2 | AAACTGGATGTTAGAGGGAAGCG | (SEQ ID NO: 148) | GGCCGACCTAGATGACTCATAAG | (SEQ ID NO: 196) |
| GAPDH | GGGTGTGAACCATGAGAA | (SEQ ID NO: 149) | GTCTTCTGGGTGGCAGTGAT | (SEQ ID NO: 197) |
| β-actin | CATGTACGTTGCTATCCAGGC | (SEQ ID NO: 150) | CTCCTTAATGTCACGCACGAT | (SEQ ID NO: 198) |

1) A method to generate induced human pluripotent stem cells comprising delivering to a somatic or non-embryonic cell population an effective amount of one or more reprogramming factors and also an agent that downmodulates SIRT2, and culturing the somatic or non-embryonic cell population for a period of time sufficient to generate at least one induced human pluripotent stem cell.

2) The method of claim 1, further comprising delivering to the somatic or non-embryonic cell population an effective amount of an agent that upmodulates SIRT1.

3) The method of claim 1, wherein the reprogramming factor is an agent that increases expression of c-Myc, Oct4, Sox2, Nanog, Lin-28, or Klf4 in the cells.

4) The method of claim 1, wherein the reprogramming factor is an agent that increases expression of SV40 Large T Antigen (“SV40LT”), or short hairpin RNAs targeting p53 (“shRNA-p53”).

5) The method of claim 1, wherein the agent that downmodulates SIRT2 is selected from the group consisting of a small molecule, an antibody, a peptide, an antisense oligonucleotide, and an RNAi.

6) The method of claim 5, wherein the RNAi is a microRNA, an siRNA, or a shRNA.

7) The method of claim 6, wherein the microRNA is miR-200c-5p.

8) The method of claim 2, wherein the agent that upmodulates SIRT1 is selected from the group consisting of a small molecule, a peptide, and an expression vector encoding SIRT1.

9) The method of claim 1, further comprising delivering to the cells one or more microRNAs selected from the miR-302/367.

10) The method of claim 1, wherein delivery comprises contacting the cell population with an agent or a vector that encodes the agent.

11) The method of claim 1, wherein delivery comprises transduction, nucleofection, electroporation, direct injection, and/or transfection.

12) The method of claim 10, wherein the vector is non-integrative or integrative.

13) The method of claim 12, wherein the non-integrative vector is selected from the group consisting of an episomal vector, an EBNA1 vector, a minicircle vector, a non-integrative adenovirus, a non-integrative RNA, and a Sendai virus.

14) The method of claim 10, wherein the vector is an episomal vector or a lentivirus vector.

15) (canceled)

16) The method of claim 1, wherein the culturing is for a period of from 7 to 21 days.

17) (canceled)

18) (canceled)

19) (canceled)

20) A cell line comprising induced pluripotent stem cells generated by the method of claim 1.

21) A pharmaceutical composition comprising an induced pluripotent stem cell or population thereof generated by the method of claim 1, and a pharmaceutically acceptable carrier.

22) A method to generate differentiated cells comprising delivering to a pluripotent cell population an agent that upmodulates SIRT2 and culturing the population under differentiating conditions for a period of time sufficient to generate at least one differentiated cell.

23) (canceled)

24) The method of claim 22, wherein the pluripotent cell population is selected from the group consisting of an embryonic stem population, an adult stem cell population, an induced pluripotent stem cell population, and a cancer stem cell population.

25)-39) (canceled)

40) A cell line comprising differentiated cells generated by the method of claim 22.

41)-49) (canceled)


SEQUENCE LISTING (220 sequences)

SEQ ID NO: 1 - RNA, 22 nt, Homo sapiens
cgucuuacccagcaguguuugg

SEQ ID NO: 2 - DNA, 759 bp, Homo sapiens
atgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagat

SEQ ID NO: 3 - DNA, 1070 bp, Homo sapiens
gcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagct

SEQ ID NO: 4 - protein, 18 aa, Homo sapiens
GVVGIKVDKGVVPLAGTN

SEQ ID NO: 5 - protein, 18 aa, Mus sp.
GVVGIKVDKGVVPLAGTN

SEQ ID NO: 6 - protein, 18 aa, Rattus sp.
GVVGIKVDKGVVPLAGTN

SEQ ID NO: 7 - protein, 18 aa, Oryctolagus sp.
GVVGIKVDKGVVPLAGTN

SEQ ID NO: 8 - protein, 18 aa, Pan sp.
GVVGIKVDKGVVPLAGTN

SEQ ID NO: 9 - protein, 18 aa, Salmo sp.
WVVGIKVDKGVVPLAGTN

SEQ ID NO: 10 - protein, 18 aa, Pongo sp.
GVVGIKVDKGVVPLAGTN

SEQ ID NO: 11 - protein, 22 aa, Artificial Sequence
  Description of Artificial Sequence Synthetic peptide
  MOD_RES (7)..(7): Acetylated-Lys
RIVAPGKGILAADESTGSIAKR

SEQ ID NO: 12 - protein, 23 aa, Artificial Sequence
  Description of Artificial Sequence Synthetic peptide
  MOD_RES (21)..(21): Acetylated-Lys
RIVAPGKGILAADESTGSIAKRL

SEQ ID NO: 13 - protein, 16 aa, Artificial Sequence
  Description of Artificial Sequence Synthetic peptide
  MOD_RES (13)..(13): Acetylated-Lys
KADDGRPFPQVIKSKG

SEQ ID NO: 14 - protein, 14 aa, Artificial Sequence
  Description of Artificial Sequence Synthetic peptide
  MOD_RES (3)..(3): Acetylated-Lys
KSKGGVVGIKVDKG

SEQ ID NO: 15 - protein, 12 aa, Artificial Sequence
  Description of Artificial Sequence Synthetic peptide
  MOD_RES (8)..(8): Acetylated-Lys
KGGVVGIKVDKG

SEQ ID NO: 16 - protein, 11 aa, Artificial Sequence
  Description of Artificial Sequence Synthetic peptide
  MOD_RES (8)..(8): Acetylated-Lys
KDGADFAKWRC

SEQ ID NO: 17 - protein, 22 aa, Artificial Sequence
  Description of Artificial Sequence Synthetic peptide
  MOD_RES (7)..(7): Acetylated-Lys
RIVAPGKGILAADESTGSIAKR

SEQ ID NO: 18 - protein, 17 aa, Artificial Sequence
  Description of Artificial Sequence Synthetic peptide
  MOD_RES (15)..(15): Acetylated-Lys
KGILAADESTGSIAKRL

SEQ ID NO: 19 - protein, 16 aa, Artificial Sequence
  Description of Artificial Sequence Synthetic peptide
  MOD_RES (13)..(13): Acetylated-Lys
KADDGRPFPQVIKSKG

SEQ ID NO: 20 - protein, 14 aa, Artificial Sequence
  Description of Artificial Sequence Synthetic peptide
  MOD_RES (3)..(3): Acetylated-Lys
KSKGGVVGIKVDKG

SEQ ID NO: 21 - protein, 14 aa, Artificial Sequence
  Description of Artificial Sequence Synthetic peptide
  MOD_RES (10)..(10): Acetylated-Lys
KSKGGVVGIKVDKG

SEQ ID NO: 22 - protein, 12 aa, Artificial Sequence
  Description of Artificial Sequence Synthetic peptide
  MOD_RES (8)..(8): Acetylated-Lys
KGGVVGIKVDKG

SEQ ID NO: 23 - protein, 28 aa, Artificial Sequence
  Description of Artificial Sequence Synthetic peptide
  MOD_RES (4)..(4): Acetylated-Lys
KVDKGVVPLAGTNGETTTQGLDGLSERC

SEQ ID NO: 24 - protein, 11 aa, Artificial Sequence
  Description of Artificial Sequence Synthetic peptide
  MOD_RES (8)..(8): Acetylated-Lys
KDGADFAKWRC

SEQ ID NO: 25 - protein, 14 aa, Artificial Sequence
  Description of Artificial Sequence Synthetic peptide
  MOD_RES (5)..(5): Acetylated-Lys
KENLKAAQEEYVKR

SEQ ID NO: 26 - RNA, 34 nt, Homo sapiens
cccucgucuuacccagcaguguuugggugcgguu

SEQ ID NO: 27 - RNA, 34 nt, Homo sapiens
gggagucucuaauacugccggguaaugauggagg

SEQ ID NO: 28 - RNA, 22 nt, Homo sapiens
cgucuuacccagcaguguuugg

SEQ ID NO: 29 - RNA, 23 nt, Homo sapiens
uaauacugccggguaaugaugga

SEQ ID NO: 30 - protein, 15 aa, Homo sapiens
RFPQLDSTSFANSRD

SEQ ID NO: 31 - protein, 27 aa, Homo sapiens
RYASIQQGIVPIVEPEILPDGDHDLKR

SEQ ID NO: 32 - protein, 17 aa, Homo sapiens
RYDGQVAVFGSDLQEKL

SEQ ID NO: 33 - protein, 13 aa, Homo sapiens
KTVLIELINVAKA

SEQ ID NO: 34 - protein, 16 aa, Homo sapiens
KVNQIGSVTESIQAKL

SEQ ID NO: 35 - protein, 14 aa, Homo sapiens
RAHSSVGVNLPQKA

SEQ ID NO: 36 - protein, 13 aa, Homo sapiens
KDSYVGDEAQSKR

SEQ ID NO: 37 - protein, 15 aa, Homo sapiens
RIVSQLLTLMDGLKQ

SEQ ID NO: 38 - protein, 13 aa, Homo sapiens
RYLAEVAAGDDKK

SEQ ID NO: 39 - protein, 17 aa, Homo sapiens
KNQVAMNPTNTVFDAKR

SEQ ID NO: 40 - protein, 17 aa, Homo sapiens
KVEGFPTIYFAPSGDKK

SEQ ID NO: 41 - protein, 15 aa, Homo sapiens
RTLTIVDTGIGMTKA

SEQ ID NO: 42 - protein, 17 aa, Homo sapiens
KHSQFIGYPITLFVEKE

SEQ ID NO: 43 - protein, 12 aa, Homo sapiens
RGYFEYIEENKY

SEQ ID NO: 44 - protein, 22 aa, Homo sapiens
KTIGGGDDSFNTFFSETGAGKH

SEQ ID NO: 45 - protein, 14 aa, Homo sapiens
RIMNTFSVVPSPKV

SEQ ID NO: 46 - protein, 14 aa, Homo sapiens
KVGGTSDVEVNEKK

SEQ ID NO: 47 - protein, 18 aa, Homo sapiens
RLNFSHGTHEYHAETIKN

SEQ ID NO: 48 - protein, 17 aa, Homo sapiens
KNQVALNPQNTVFDAKR

SEQ ID NO: 49 - protein, 16 aa, Homo sapiens
KGILAADESTGSIAKR

SEQ ID NO: 50 - protein, 17 aa, Homo sapiens
RGALQNIIPASTGAAKA

SEQ ID NO: 51 - protein, 18 aa, Homo sapiens
RIINEPTAAAIAYGLDKR

SEQ ID NO: 52 - protein, 15 aa, Homo sapiens
KVTQDATPGSALDKI

SEQ ID NO: 53 - protein, 13 aa, Homo sapiens
RSLGTADVHFERK

SEQ ID NO: 54 - protein, 13 aa, Homo sapiens
KEDVFVHQTAIKK

SEQ ID NO: 55 - protein, 15 aa, Homo sapiens
RMVIITGPPEAQFKA

SEQ ID NO: 56 - protein, 18 aa, Homo sapiens
KFNISNGGPAPEAITDKI

SEQ ID NO: 57 - protein, 15 aa, Homo sapiens
KVEITYTPSDGTQKV

SEQ ID NO: 58 - protein, 16 aa, Homo sapiens
RILAQATSDLVNAIKA

SEQ ID NO: 59 - protein, 19 aa, Homo sapiens
KVLAVNQENEQLMEDYEKL

SEQ ID NO: 60 - protein, 15 aa, Homo sapiens
KWCGTTQNYDADQKF

SEQ ID NO: 61 - protein, 20 aa, Homo sapiens
KLSLEGDHSTPPSAYGSVKA

SEQ ID NO: 62 - protein, 21 aa, Homo sapiens
RPVTVEPMDQLDDEEGLPEKL

SEQ ID NO: 63 - protein, 15 aa, Homo sapiens
REDSQRPGAHLTVKK

SEQ ID NO: 64 - protein, 17 aa, Homo sapiens
RLTEMETLQSQLMAEKL

SEQ ID NO: 65 - protein, 14 aa, Homo sapiens
RPANDFNPDADAKA

SEQ ID NO: 66 - protein, 15 aa, Homo sapiens
REESGKPGAHVTVKK

SEQ ID NO: 67 - protein, 15 aa, Homo sapiens
KLLATEQEDAAVAKS

SEQ ID NO: 68 - protein, 16 aa, Homo sapiens
KSINDNIAIFTEVQKR

SEQ ID NO: 69 - protein, 13 aa, Homo sapiens
KAAYEAELGDARK

SEQ ID NO: 70 - protein, 15 aa, Homo sapiens
KSDDEVDDPAVELKQ

SEQ ID NO: 71 - protein, 20 aa, Homo sapiens
KGAGTGGLGLTVEGPCEAKI

SEQ ID NO: 72 - protein, 15 aa, Homo sapiens
RFPQLDSTSFANSRD

SEQ ID NO: 73 - protein, 16 aa, Homo sapiens
RILAQATSDLVNAIKA

SEQ ID NO: 74 - protein, 22 aa, Artificial Sequence
  Description of Artificial Sequence Synthetic peptide
  MOD_RES (7)..(7): Acetylated-Lys
RIVAPGKGILAADESTGSIAKR

SEQ ID NO: 75 - protein, 23 aa, Artificial Sequence
  Description of Artificial Sequence Synthetic peptide
  MOD_RES (21)..(21): Acetylated-Lys
RIVAPGKGILAADESTGSIAKRL

SEQ ID NO: 76 - protein, 16 aa, Artificial Sequence
  Description of Artificial Sequence Synthetic peptide
  MOD_RES (13)..(13): Acetylated-Lys
KADDGRPFPQVIKSKG

SEQ ID NO: 77 - protein, 14 aa, Artificial Sequence
  Description of Artificial Sequence Synthetic peptide
  MOD_RES (3)..(3): Acetylated-Lys
KSKGGVVGIKVDKG

SEQ ID NO: 78 - protein, 12 aa, Artificial Sequence
  Description of Artificial Sequence Synthetic peptide
  MOD_RES (8)..(8): Acetylated-Lys
KGGVVGIKVDKG

SEQ ID NO: 79 - protein, 11 aa, Artificial Sequence
  Description of Artificial Sequence Synthetic peptide
  MOD_RES (8)..(8): Acetylated-Lys
KDGADFAKWRC

SEQ ID NO: 80 - protein, 22 aa, Artificial Sequence
  Description of Artificial Sequence Synthetic peptide
  MOD_RES (7)..(7): Acetylated-Lys
RIVAPGKGILAADESTGSIAKR

SEQ ID NO: 81 - protein, 17 aa, Artificial Sequence
  Description of Artificial Sequence Synthetic peptide
  MOD_RES (15)..(15): Acetylated-Lys
KGILAADESTGSIAKRL

SEQ ID NO: 82 - protein, 16 aa, Artificial Sequence
  Description of Artificial Sequence Synthetic peptide
  MOD_RES (13)..(13): Acetylated-Lys
KADDGRPFPQVIKSKG

SEQ ID NO: 83 - protein, 14 aa, Artificial Sequence
  Description of Artificial Sequence Synthetic peptide
  MOD_RES (3)..(3): Acetylated-Lys
KSKGGVVGIKVDKG

SEQ ID NO: 84 - protein, 14 aa, Artificial Sequence
  Description of Artificial Sequence Synthetic peptide
  MOD_RES (10)..(10): Acetylated-Lys
KSKGGVVGIKVDKG

SEQ ID NO: 85 - protein, 12 aa, Artificial Sequence
  Description of Artificial Sequence Synthetic peptide
  MOD_RES (8)..(8): Acetylated-Lys
KGGVVGIKVDKG

SEQ ID NO: 86 - protein, 28 aa, Artificial Sequence
  Description of Artificial Sequence Synthetic peptide
  MOD_RES (4)..(4): Acetylated-Lys
KVDKGVVPLAGTNGETTTQGLDGLSERC

SEQ ID NO: 87 - protein, 11 aa, Artificial Sequence
  Description of Artificial Sequence Synthetic peptide
  MOD_RES (8)..(8): Acetylated-Lys
KDGADFAKWRC

SEQ ID NO: 88 - protein, 14 aa, Artificial Sequence
  Description of Artificial Sequence Synthetic peptide
  MOD_RES (5)..(5): Acetylated-Lys
KENLKAAQEEYVKR

SEQ ID NO: 89 - DNA, 22 bp, Homo sapiens
aagcgcgtctgcggccgcaatg

SEQ ID NO: 90 - DNA, 22 bp, Homo sapiens
aagcgcgtctgcggccgcaatg

SEQ ID NO: 91 - DNA, 22 bp, Homo sapiens
tccccgccacctgccaaggacg

SEQ ID NO: 92 - DNA, 22 bp, Homo sapiens
acaggaggacttggtggaggcg

SEQ ID NO: 93 - DNA, 22 bp, Homo sapiens
atgtctgctgagagttgtagtt

SEQ ID NO: 94 - DNA, 22 bp, Homo sapiens
cccaggcagggaaggtgcagga

SEQ ID NO: 95 - DNA, 22 bp, Homo sapiens
gtacctccttgcaggtgcagcc

SEQ ID NO: 96 - DNA, 22 bp, Homo sapiens
cattgcacttgtctcggtctga

SEQ ID NO: 97 - DNA, 22 bp, Homo sapiens
tattgcactcgtcccggcctcc

SEQ ID NO: 98 - DNA, 22 bp, Homo sapiens
cgtcttacccagcagtgtttgg

SEQ ID NO: 99 - DNA, 22 bp, Homo sapiens
cgtcttacccagcagtgtttgg

SEQ ID NO: 100 - DNA, 22 bp, Homo sapiens
aattgcactttagcaatggtga

SEQ ID NO: 101 - DNA, 22 bp, Homo sapiens
aattgcactttagcaatggtga

SEQ ID NO: 102 - DNA, 22 bp, Homo sapiens
aattgcactttagcaatggtga

SEQ ID NO: 103 - DNA, 22 bp, Artificial Sequence
  Description of Artificial Sequence Synthetic primer
tagacacgctggaacaggttgc

SEQ ID NO: 104 - DNA, 23 bp, Artificial Sequence
  Description of Artificial Sequence Synthetic primer
ctgcggaacttattctcccagac

SEQ ID NO: 105 - DNA, 20 bp, Artificial Sequence
  Description of Artificial Sequence Synthetic primer
cattccagacttcagatcgc

SEQ ID NO: 106 - DNA, 21 bp, Artificial Sequence
  Description of Artificial Sequence Synthetic primer
tgggatcatccttgcaggtat

SEQ ID NO: 107 - DNA, 21 bp, Artificial Sequence
  Description of Artificial Sequence Synthetic primer
gccaagttcaagtatggcaga

SEQ ID NO: 108 - DNA, 22 bp, Artificial Sequence
  Description of Artificial Sequence Synthetic primer
tggcagtcttccagtgtggtgt

SEQ ID NO: 109 - DNA, 22 bp, Artificial Sequence
  Description of Artificial Sequence Synthetic primer
tggagtgtggacactgcttcag

SEQ ID NO: 110 - DNA, 21 bp, Artificial Sequence
  Description of Artificial Sequence Synthetic primer
caaggcatcctttgaggtctc

SEQ ID NO: 111 - DNA, 20 bp, Artificial Sequence
  Description of Artificial Sequence Synthetic primer
caacagcacggccatccagg

SEQ ID NO: 112 - DNA, 22 bp, Artificial Sequence
  Description of Artificial Sequence Synthetic primer
gagtacaccgccgaggagattg

SEQ ID NO: 113 - DNA, 20 bp, Artificial Sequence
  Description of Artificial Sequence Synthetic primer
gctcgagaaggatgtggtcc

SEQ ID NO: 114 - DNA, 20 bp, Artificial Sequence
  Description of Artificial Sequence Synthetic primer
aaccccaagatgcacaactc

SEQ ID NO: 115 - DNA, 20 bp, Artificial Sequence
  Description of Artificial Sequence Synthetic primer
caaaggcaaacaacccactt

SEQ ID NO: 116 - DNA, 20 bp, Artificial Sequence
  Description of Artificial Sequence Synthetic primer
tgtcaagccatgatggaaaa

SEQ ID NO: 117 - DNA, 20 bp, Artificial Sequence
  Description of Artificial Sequence Synthetic primer
ggcggaaatagaacctgtca

SEQ ID NO: 118 - DNA, 18 bp, Artificial Sequence
  Description of Artificial Sequence Synthetic primer
gtccccaccgaagtctgc

SEQ ID NO: 119 - DNA, 20 bp, Artificial Sequence
  Description of Artificial Sequence Synthetic primer
aaatgtttgtgttgcggtca

SEQ ID NO: 120 - DNA, 20 bp, Artificial Sequence
  Description of Artificial Sequence Synthetic primer
gcctgggagaagttcgtgta

SEQ ID NO: 121 - DNA, 20 bp, Artificial Sequence
  Description of Artificial Sequence Synthetic primer
cgaaggtagttcgccttgag

SEQ ID NO: 122 - DNA, 20 bp, Artificial Sequence
  Description of Artificial Sequence Synthetic primer
ggtgcaggtgaaaatctggt

SEQ ID NO: 123 - DNA, 28 bp, Artificial Sequence
  Description of Artificial Sequence Synthetic primer
acccattatccagatgtgtttgcccgag

SEQ ID NO: 124 - DNA, 30 bp, Artificial Sequence
  Description of Artificial Sequence Synthetic primer
caggtggcggacgtgtgaaaattgagagtg

SEQ ID NO: 125 - DNA, 26 bp, Artificial Sequence
  Description of Artificial Sequence Synthetic primer
ggcccgccacttgcaggagtaccagg

SEQ ID NO: 126 - DNA, 26 bp, Artificial Sequence
  Description of Artificial Sequence Synthetic primer
cgccaggatccccgctttgaaatctg

SEQ ID NO: 127 - DNA, 26 bp, Artificial Sequence
  Description of Artificial Sequence Synthetic primer
tgggagcggtgaagatggaagggcac

SEQ ID NO: 128 - DNA, 26 bp, Artificial Sequence
  Description of Artificial Sequence Synthetic primer
cgctttcatggtgtgggctaaggacg

SEQ ID NO: 129 - DNA, 28 bp, Artificial Sequence
  Description of Artificial Sequence Synthetic primer
gaatgctgcaaactgaccacgctggaac

SEQ ID NO: 130 - DNA, 27 bp, Artificial Sequence
  Description of Artificial Sequence Synthetic primer
cctggaagggctgaccgacgagatcaa

SEQ ID NO: 131 - DNA, 27 bp, Artificial Sequence
  Description of Artificial Sequence Synthetic primer
agctcaacgggatcctgctgcaccttg

SEQ ID NO: 132 - DNA, 24 bp, Artificial Sequence
  Description of Artificial Sequence Synthetic primer
cgagaggaccccgtggatgcagag

SEQ ID NO: 133 - DNA, 26 bp, Artificial Sequence
  Description of Artificial Sequence Synthetic primer
gccctctccctcccctccacgcacag

SEQ ID NO: 134 - DNA, 20 bp, Artificial Sequence
  Description of Artificial Sequence Synthetic primer
tggcatcaacgctgtcttct

SEQ ID NO: 135 - DNA, 20 bp, Artificial Sequence
  Description of Artificial Sequence Synthetic primer
gctgcgaataaacaggcagg

SEQ ID NO: 136 - DNA, 22 bp, Artificial Sequence
  Description of Artificial Sequence Synthetic primer
gacccagagatgctgtaatggt

SEQ ID NO: 137 - DNA, 20 bp, Artificial Sequence
  Description of Artificial Sequence Synthetic primer
attgctcatgcccctactca

SEQ ID NO: 138 - DNA, 20 bp, Artificial Sequence
  Description of Artificial Sequence Synthetic primer
gcatgaaggaagggaggctg

SEQ ID NO: 139 - DNA, 20 bp, Artificial Sequence
  Description of Artificial Sequence Synthetic primer
tctcagcggccatcatgttt

SEQ ID NO: 140 - DNA, 21 bp, Artificial Sequence
  Description of Artificial Sequence Synthetic primer
cattccattgggcccagtcct

SEQ ID NO: 141 - DNA, 21 bp, Artificial Sequence
  Description of Artificial Sequence Synthetic primer
cagggacaaatggcgagacta

SEQ ID NO: 142 - DNA, 21 bp, Artificial Sequence
  Description of Artificial Sequence Synthetic primer
tgtctggtggcatgagtgaag

SEQ ID NO: 143 - DNA, 21 bp, Artificial Sequence
  Description of Artificial Sequence Synthetic primer
gccaaattggggtggaaaaca

SEQ ID NO: 144 - DNA, 20 bp, Artificial Sequence
  Description of Artificial Sequence Synthetic primer
gccgtgaacgagaagtcctg

SEQ ID NO: 145 - DNA, 20 bp, Artificial Sequence
  Description of Artificial Sequence Synthetic primer
ccgggaactcagacctcatc

SEQ ID NO: 146 - DNA, 23 bp, Artificial Sequence
  Description of Artificial Sequence Synthetic primer
tatcgcaatgggaagtacgatct

SEQ ID NO: 147 - DNA, 21 bp, Artificial Sequence
  Description of Artificial Sequence Synthetic primer
gaacaaggttaaagccgagcc

SEQ ID NO: 148 - DNA, 23 bp, Artificial Sequence
  Description of Artificial Sequence Synthetic primer
aaactggatgttagagggaagcg

SEQ ID NO: 149 - DNA, 18 bp, Artificial Sequence
  Description of Artificial Sequence Synthetic primer
gggtgtgaaccatgagaa

SEQ ID NO: 150 - DNA, 21 bp, Artificial Sequence
  Description of Artificial Sequence Synthetic primer
catgtacgttgctatccaggc

SEQ ID NO: 151 - DNA, 22 bp, Artificial Sequence
  Description of Artificial Sequence Synthetic primer
ctcctcgtacagcttcacagtc

SEQ ID NO: 152 - DNA, 22 bp, Artificial Sequence
  Description of Artificial Sequence Synthetic primer
ccaccaaacagatgactctgcg

SEQ ID NO: 153 - DNA, 19 bp, Artificial Sequence
  Description of Artificial Sequence Synthetic primer
agcagccggagaaagtagt

SEQ ID NO: 154 - DNA, 20 bp, Artificial Sequence
  Description of Artificial Sequence Synthetic primer
tggtcagcatgggtctatca

SEQ ID NO: 155 - DNA, 17 bp, Artificial Sequence
  Description of Artificial Sequence Synthetic primer
cgccggtagtggtagaa

SEQ ID NO: 156 - DNA, 22 bp, Artificial Sequence
  Description of Artificial Sequence Synthetic primer
cgctctcaaaggtggtgtcgaa

SEQ ID NO: 157 - DNA, 22 bp, Artificial Sequence
  Description of Artificial Sequence Synthetic primer
ccgtcacagttctgagacacca

SEQ ID NO: 158 - DNA, 20 bp, Artificial Sequence
  Description of Artificial Sequence Synthetic primer
tccatgcggcttgacagaac

SEQ ID NO: 159 - DNA, 21 bp, Artificial Sequence
  Description of Artificial Sequence Synthetic primer
cttggggccctgggcctccga

SEQ ID NO: 160 - DNA, 22 bp, Artificial Sequence
  Description of Artificial Sequence Synthetic primer
gcggatatactgggtgcactgg

SEQ ID NO: 161 - DNA, 20 bp, Artificial Sequence
  Description of Artificial Sequence Synthetic primer
cgttgtgcatagtcgctgct

SEQ ID NO: 162 - DNA, 20 bp, Artificial Sequence
  Description of Artificial Sequence Synthetic primer
cggggccggtatttataatc

SEQ ID NO: 163 - DNA, 20 bp, Artificial Sequence
  Description of Artificial Sequence Synthetic primer
tctgctggaggctgaggtat

SEQ ID NO: 164 - DNA, 20 bp, Artificial Sequence
  Description of Artificial Sequence Synthetic primer
ggtgagccagagatgctttc

SEQ ID NO: 165 - DNA, 20 bp, Artificial Sequence
  Description of Artificial Sequence Synthetic primer
cttccaggatgggttgagaa

SEQ ID NO: 166 - DNA, 20 bp, Artificial Sequence
  Description of Artificial Sequence Synthetic primer
ggacactgtctggtcgaagg

SEQ ID NO: 167 - DNA, 20 bp, Artificial Sequence
  Description of Artificial Sequence Synthetic primer
tctggcacaggtgtcttcag

SEQ ID NO: 168 - DNA, 20 bp, Artificial Sequence
  Description of Artificial Sequence Synthetic primer
actaagcgccagaaactgga

SEQ ID NO: 169 - DNA, 20 bp, Artificial Sequence
  Description of Artificial Sequence Synthetic primer
cggtgatagtcagccaggtt

SEQ ID NO: 170 - DNA, 20 bp, Artificial Sequence
  Description of Artificial Sequence Synthetic primer
gctgctgatgctgacttctg

SEQ ID NO: 171 - DNA, 26 bp, Artificial Sequence
  Description of Artificial Sequence Synthetic primer
atggtgaagctgggcataggcggcag

SEQ ID NO: 172 - DNA, 26 bp, Artificial Sequence
  Description of Artificial Sequence Synthetic primer
cacgctggatctgcctggggactgtg

SEQ ID NO: 173 - DNA, 26 bp, Artificial Sequence
  Description of Artificial Sequence Synthetic primer
cttctgctcgggcccctcatgagacg

SEQ ID NO: 174 - DNA, 26 bp, Artificial Sequence
  Description of Artificial Sequence Synthetic primer
tcggccgccagctctttgatgtgttc

SEQ ID NO: 175 - DNA, 26 bp, Artificial Sequence
  Description of Artificial Sequence Synthetic primer
tcatgccagcgcccacgtacgacgac

SEQ ID NO: 176 - DNA, 26 bp, Artificial Sequence
  Description of Artificial Sequence Synthetic primer
tagttggggtggtcctgcatgtgctg

SEQ ID NO: 177 - DNA, 28 bp, Artificial Sequence
  Description of Artificial Sequence Synthetic primer
tggcattcaagagggttttcagtctgga

SEQ ID NO: 178 - DNA, 25 bp, Artificial Sequence
  Description of Artificial Sequence Synthetic primer
cttcccagccaggctctgcagctcc

SEQ ID NO: 179 - DNA, 27 bp, Artificial Sequence
  Description of Artificial Sequence Synthetic primer
cactatccggcgggtggtggtcttttg

SEQ ID NO: 180 - DNA, 24 bp, Artificial Sequence
  Description of Artificial Sequence Synthetic primer
ggcggccatcttcagcttctccag

SEQ ID NO: 181 - DNA, 26 bp, Artificial Sequence
  Description of Artificial Sequence Synthetic primer
cggcgccgttgctcacagaccacagg

SEQ ID NO: 182 - DNA, 20 bp, Artificial Sequence
  Description of Artificial Sequence Synthetic primer
aacagcgacacgacagtgaa

SEQ ID NO: 183 - DNA, 21 bp, Artificial Sequence
  Description of Artificial Sequence Synthetic primer
agggtcccagtgaccttatct

SEQ ID NO: 184 - DNA, 20 bp, Artificial Sequence
  Description of Artificial Sequence Synthetic primer
ggggtgaccttctgtgtccc

SEQ ID NO: 185 - DNA, 20 bp, Artificial Sequence
  Description of Artificial Sequence Synthetic primer
cctggtgaagagtgccccta

SEQ ID NO: 186 - DNA, 20 bp, Artificial Sequence
  Description of Artificial Sequence Synthetic primer
acagaccacagcaacgtcaa

SEQ ID NO: 187 - DNA, 20 bp, Artificial Sequence
  Description of Artificial Sequence Synthetic primer
ggcgtagcccatgatgaaga

SEQ ID NO: 188 - DNA, 21 bp, Artificial Sequence
  Description of Artificial Sequence Synthetic primer
tgaaactgtaggcaccgatgg

SEQ ID NO: 189 - DNA, 20 bp, Artificial Sequence
  Description of Artificial Sequence Synthetic primer
ggggtgtgttccccaatctt

SEQ ID NO: 190 - DNA, 22 bp, Artificial Sequence
  Description of Artificial Sequence Synthetic primer
ggcccgtccataagagaaactt

SEQ ID NO: 191 - DNA, 21 bp, Artificial Sequence
  Description of Artificial Sequence Synthetic primer
ttcacacggtcatcagcactg

SEQ ID NO: 192 - DNA, 19 bp, Artificial Sequence
  Description of Artificial Sequence Synthetic primer
acgcctgaagagactcggt

SEQ ID NO: 193 - DNA, 20 bp, Artificial Sequence
  Description of Artificial Sequence Synthetic primer
ctctgcacctagtcgcatgg

SEQ ID NO: 194 - DNA, 22 bp, Artificial Sequence
  Description of Artificial Sequence Synthetic primer
aagctcttatacagctctccga

SEQ ID NO: 195 - DNA, 21 bp, Artificial Sequence
  Description of Artificial Sequence Synthetic primer
gtggcagattgactcctacca

SEQ ID NO: 196 - DNA, 23 bp, Artificial Sequence
  Description of Artificial Sequence Synthetic primer
ggccgacctagatgactcataag

SEQ ID NO: 197 - DNA, 20 bp, Artificial Sequence
  Description of Artificial Sequence Synthetic primer
gtcttctgggtggcagtgat

SEQ ID NO: 198 - DNA, 21 bp, Artificial Sequence
  Description of Artificial Sequence Synthetic primer
ctccttaatgtcacgcacgat

SEQ ID NO: 199 - DNA, 11943 bp, Artificial Sequence
  Description of Artificial Sequence Synthetic polynucleotide
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctctctcttgaaaacaaaaaggaaacataagaaaaaacataaagagagacataaaatgggagaagaagttataccattaagagtgctatcaaagtaagtctgtggtttaaattctgtcattggcttaacaatccatcaccattgctaaagtgcaattccaatttatattcaacagagttgcatattagcaacagtaatggcctgtagccaagaactgcacacagtgtgggcgttaacgcaattgctgattaggtaggaaccaccacactcaaacatggaagcacttatttttgtcatgtcacagcaagtgcctccatgttaaagtagagggggccccttaacagatgtaaaaatacaaaataaagcttaaatatatgagctgcggtcaatacaataaagttattttctaaaaaaataataaaaatgtaaaaggaagacttaccatcaccaaaacatggaagcacttacttctttagtttcaaagcaagtacatccacgtttaagtggtggggagcccagtcttggaaaaagttagaatcctttaacctgtaacaagcttagtaaccttaaaacacaataacaaaaaaattttgtaacaaaaggtgtgctggcttggagacacctccactgaaacatggaagcacttacttttgtttcacacagcaggtacccccatgttaaagcaaaggggatcccttcaaatgaggttagcgtgttctattttggagaagtaaattggattcactcctactaaaacatggaagcacttacttttaaagtcacagaaagcacttccatgttaaagttgaagggagcccacccaacatacaacttctttggacttcagagtatttagagctgaggagaaagaaaacaaaatggcataactttagaagaaaaaaatttttttaccttcctgaactagttcccaaagattcgtgttctcctccagaagggtaaaaggcagggacttcagccacttctatttatactattcttaactctttctagaagggctcaccaggaagtgtccccagggactcgggtggtggggggatgggagccagggatctgcagcttttccgcagggatcctgggcctgaagctgcctgacccaaggtgggcgggctgggcgggggccctcgtcttacccagcagtgtttgggtgcggttgggagtctctaatactgccgggtaatgatggaggcccctgtccctgtgtcagcaacatccatcgcctcaggtccccagcccttagctggctgcagccccctccccacttcccacgcaccccggaagcccctcgtcttgagctgagagcgttgcacaaggggtggttcttgttggctggctgccactaagggacacaatgggccccagcccctcctcccacccagtgcgatttgtcacctggtggatccagaacccacagtcgaccttgagcttggggttggctcgccccctctcaagagacctcacctggcctgtggccagggtcccctgtagcaactggtgagcgcgcaccgtagttctctgtcggccggccctgggtccatcttccagtacagtgttggatggtctaattgtgaagctcctaacactgtctggtaaagatggctcccgggtgggttctctcggcagtaaccttcagggagccctgaagaccatggaggactactgaccaacaacctctgaccttcacccctctggatgggggacgaatcactaggcaaaggggaacaatgggaaggagacagaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 200 - RNA, 7 nt, Homo sapiens
aagugcu

SEQ ID NO: 201 - protein, 16 aa, Homo sapiens
GGKKENLKAAQEEYVK

SEQ ID NO: 202 - protein, 16 aa, Mus sp.
GGKKENLKAAQEEYIK

SEQ ID NO: 203 - protein, 16 aa, Rattus sp.
GGKKENLKAAQEEYIK

SEQ ID NO: 204 - protein, 16 aa, Oryctolagus sp.
GGKKENLKAAQEEYVK

SEQ ID NO: 205 - protein, 16 aa, Pan sp.
GGKKENLKAAQEEYVK

SEQ ID NO: 206 - protein, 16 aa, Salmo sp.
GGKPGNGKAAQEEFIK

SEQ ID NO: 207 - protein, 16 aa, Pongo sp.
GGKKENLKAAQEEYVK

SEQ ID NO: 208 - RNA, 68 nt, Homo sapiens
cccucgucuuacccagcaguguuugggugcgguugggagucucuaauacugccggguaaugauggagg

SEQ ID NO: 209 - protein, 9 aa, Homo sapiens
EIIDLVLDR

SEQ ID NO: 210 - protein, 12 aa, Homo sapiens
PYQYPALTPEQK

SEQ ID NO: 211 - protein, 8 aa, Homo sapiens
VGVNGFGR

SEQ ID NO: 212 - protein, 15 aa, Homo sapiens
LGDVYVNDAFGTAHR

SEQ ID NO: 213 - protein, 14 aa, Homo sapiens
VNQIGSVTESLQAC

SEQ ID NO: 214 - protein, 14 aa, Homo sapiens
NQIGSVTESLQACK

SEQ ID NO: 215 - protein, 9 aa, Homo sapiens
GDYPLEAVR

SEQ ID NO: 216 - protein, 10 aa, Homo sapiens
SFLSQGQVLK

SEQ ID NO: 217 - protein, 23 aa, Homo sapiens
VDKGVVPLAGTNGETTTQGLDGL

SEQ ID NO: 218 - protein, 24 aa, Artificial Sequence
  Description of Artificial Sequence Synthetic peptide
  MOD_RES (3)..(3): Acetylated-Lys
VDKGVVPLAGTNGETTTQGLDGLS

SEQ ID NO: 219 - protein, 13 aa, Homo sapiens
ENLKAAQEEYVKR

SEQ ID NO: 220 - protein, 12 aa, Artificial Sequence
  Description of Artificial Sequence Synthetic peptide
  MOD_RES (4)..(4): Acetylated-Lys
ENLKAAQEEYVK